(19) United States
(12) Patent Application Publication
(10) Pub. No.: US 2015/0306238 A1
(43) Pub. Date: Oct. 29, 2015

(54) BACTERIA TARGETING NANOPARTICLES AND RELATED METHODS OF USE

(71) Applicant: THE REGENTS OF THE UNIVERSITY OF MICHIGAN, Ann Arbor, MI (US)

(72) Inventors: James R. Baker, Ann Arbor, MI (US); Seok Ki Choi, Ann Arbor, MI (US); Andrzej Myc, Ann Arbor, MI (US)

(21) Appl. No.: 14/651,579

(22) PCT Filed: Nov. 27, 2013
(86) PCT No.: PCT/US2013/072363

§ 371 Date: Jun. 11, 2015

Related U.S. Application Data
(60) Provisional application No. 61/736,225, filed on Dec. 12, 2012.

(51) Int. Cl. A61K 47/48; A61K 45/06; A61K 38/12; C12Q 1/04

(52) U.S. Cl. CPC A61K 47/48192; C12Q 1/04; A61K 45/06; A61K 38/12

(57) ABSTRACT

The present invention relates to bacteria-targeting nanoparticles and related methods of use. In particular, the present invention relates to dendrimer nanoparticles conjugated with Vancomycin and/or Polymixin (e.g., Polymixin B, Polymixin E). In certain embodiments, such dendrimer nanoparticles are used to sequester and/or identify bacteria (e.g., Gram-positive bacteria and/or Gram-negative bacteria), screen liquid samples (e.g., water samples, food samples, pharmaceutical samples, blood samples, blood platelet samples, etc.) for the presence of bacteria (e.g., Gram-positive bacteria and/or Gram-negative bacteria), treat disorders associated with bacteria (e.g., Gram-positive bacteria and/or Gram-negative bacteria), and/or identify, sequester, and remove bacteria (e.g., Gram-positive bacteria and/or Gram-negative bacteria) from a liquid sample (e.g., water sample, food sample, pharmaceutical sample, blood sample, blood platelet sample, etc.). In certain embodiments, iron oxide nanoparticles are coated with dendrimer nano-particles conjugated with Vancomycin and/or Polymixin.

### CROSS-REFERENCE TO RELATED APPLICATIONS

This application claims priority to pending U.S. Provisional Patent Application No. 61/736,225, filed Dec. 12, 2012, the contents of which are incorporated by reference in their entirety.

### FIELD OF THE INVENTION

The present invention relates to bacteria-targeting nanoparticles and related methods of use. In particular, the present invention relates to dendrimer nanoparticles conjugated with Vancomycin and/or Polymixin (e.g., Polymixin B, Polymixin E). In certain embodiments, such dendrimer nanoparticles are used to sequester and/or identify bacteria (e.g., Gram-positive bacteria and/or Gram-negative bacteria), screen liquid samples (e.g., water samples, food samples, pharmaceutical samples, blood samples, blood platelet samples, etc.) for the presence of bacteria (e.g., Gram-positive bacteria and/or Gram-negative bacteria), treat disorders associated with bacteria (e.g., Gram-positive bacteria and/or Gram-negative bacteria), and/or identify, sequester, and remove bacteria (e.g., Gram-positive bacteria and/or Gram-negative bacteria) from a liquid sample (e.g., water sample, food sample, pharmaceutical sample, blood sample, blood platelet sample, etc.). In certain embodiments, iron oxide nanoparticles are coated with dendrimer nanoparticles conjugated with Vancomycin and/or Polymixin (e.g., Polymixin B, Polymixin E).

### BACKGROUND OF THE INVENTION

Gram-positive and Gram-negative bacterial infections (see, e.g., Bihl, F.; et al., J. Transl. Med. 2007, 5, 25; Depcik-Smith, N. D.; et al., J. Clinical Apheresis 2001, 16, 192-201; each herein incorporated by reference in its entirety)) cause numerous serious medical conditions including sepsis, bacteremia, pneumonia and endocarditis (see, e.g., Jacoby, G. A.; et al., N. Engl. J. Med. 1991, 324, 601-612; Dajani, A. S.; et al., Circulation 1997, 96, 358-366; Tipple, M. A.; et al., Transfusion (Malden, Mass., U.S.) 1990, 30, 207-213; Muder, R. R.; et al., Transfusion (Malden, Mass., U.S.) 1992, 32, 771-774; each herein incorporated by reference in its entirety)). Such infections can be life-threatening, especially when associated with drug-resistant bacteria (see, e.g., Jacoby, G. A.; et al., N. Engl. J. Med. 1991, 324, 601-612; Cosgrove, S. E.; et al., Clin. Infect. Dis. 2003, 36, 53-59; each herein incorporated by reference in its entirety)). Despite the fact that there are growing concerns about serious infectious diseases, current technology is not able to fully and accurately detect, enumerate, or treat those causative bacterial cells inhabiting the blood and other vital organs (see, e.g., Schmidt, M.; et al., Transfusion Medicine and Hemotherapy 2011, 38, 259-265; herein incorporated by reference in its entirety)).

Improved methods and techniques for treating bacterial infections (e.g., Gram-positive bacterial infections and/or Gram-negative bacterial infections) are needed.

### SUMMARY OF THE INVENTION

Gram-positive and Gram-negative bacterial infections (see, e.g., Bihl, F.; et al., J. Transl. Med. 2007, 5, 25; Depcik-Smith, N. D.; et al., J. Clinical Apheresis 2001, 16, 192-201; each herein incorporated by reference in its entirety)) cause numerous serious medical conditions including sepsis, bacteremia, pneumonia and endocarditis (see, e.g., Jacoby, G. A.; et al., N. Engl. J. Med. 1991, 324, 601-612; Dajani, A. S.; et al., Circulation 1997, 96, 358-366; Tipple, M. A.; et al., Transfusion (Malden, Mass., U.S.) 1990, 30, 207-213; Muder, R. R.; et al., Transfusion (Malden, Mass., U.S.) 1992, 32, 771-774; each herein incorporated by reference in its entirety)). Such infections can be life-threatening, especially when associated with drug-resistant bacteria (see, e.g., Jacoby, G. A.; et al., N. Engl. J. Med. 1991, 324, 601-612; Cosgrove, S. E.; et al., Clin. Infect. Dis. 2003, 36, 53-59; each herein incorporated by reference in its entirety)). Despite the fact that there are growing concerns about serious infectious diseases, current technology is not able to fully and accurately detect, enumerate, or treat those causative bacterial cells inhabiting the blood and other vital organs (see, e.g., Schmidt, M.; et al., Transfusion Medicine and Hemotherapy 2011, 38, 259-265; herein incorporated by reference in its entirety)).

The present invention provides a novel nanotechnology that supports the right combination of higher sensitivity, speed and ease of the assay for detecting bacteria in aqueous samples (see, e.g., FIG. 1). Experiments conducted during the course of developing embodiments for the present invention involved a biophysical evaluation and practical exploration of vancomycin-presenting, poly(amidoamine) (PAMAM) dendrimers as a platform enabling detection and isolation of bacterial pathogens. Vancomycin represents a preferred ligand for bacteria-targeting nanosystems. However, it is inefficient for emerging vancomycin-resistant species because of its poor affinity to the reprogrammed cell wall structure. The present invention demonstrates the use of a multivalent strategy as an effective way for overcoming affinity limitations present in bacteria targeting. In experiments conducted during the course of developing embodiments for the present invention, a series of fifth generation (G5) poly(amidoamine) (PAMAM) dendrimers tethered with vancomycin at the C-terminus at different valencies were designed. Surface plasmon resonance (SPR) studies was performed to determine their binding avidity to two cell wall models, each made with either a vancomycin-susceptible (D)-Ala-(D)-Ala or vancomycin-resistant (D)-Ala-(D)-Lac cell wall precursor. These conjugates showed remarkable enhancement in avidity in the cell wall models tested, including the vancomycin-resistant model, which had an increase in avidity of four to five orders of magnitude greater than free vancomycin. The tight adsorption of the conjugate to the model surface corresponded with its ability to bind vancomycin-susceptible Staphylococcus aureus bacterial cells in vitro as imaged by confocal fluorescent microscopy. This vancomycin platform was then used to fabricate the surface of iron oxide nanoparticles by coating them with the dendrimer conjugates, and the resulting dendrimer-covered magnetic nanoparticles were demonstrated to rapidly sequester bacterial cells.

The present invention provides a novel nanotechnology that supports the right combination of higher sensitivity, speed and ease of the assay for detecting bacteria in aqueous samples (see, e.g., FIG. 1). Indeed, embodiments of the present invention represent a significant technological advance, with the inherent modularity of the nanoparticle technology providing many options for rapid, sensitive, and portable screening of bacterial contaminants from blood and blood products. The clinical significance of this work is considerable, as a significant outcome is a reduction in the number of adverse events suffered by patients as a result of transfusion-related sepsis. Indeed, preventative interventions that are so critical to transfusion medicine are enhanced by the technology that monitors bacterial contamination of platelets. In addition, the methods of the present invention allow the separation of Gram-positive and/or Gram-negative bacteria from a sample (e.g., a food sample, blood sample, blood product sample, water sample, pharmaceutical sample) without the need for chemical treatment of the sample.

Accordingly, the present invention provides compositions comprising dendrimer nanoparticles conjugated with Vancomycin molecules and/or Polymyxin (e.g., Polymyxin B, Polymyxin E) molecules. In certain embodiments, the dendrimer nanoparticles are conjugated with two or more Vancomycin molecules and/or Polymyxin B or E molecules (e.g., conjugated with an average of 2.3, 3.5 or 5.8 Vancomycin and/or Polymyxin molecules)). In some embodiments, iron oxide nanoparticles are coated with such dendrimer nanoparticles.

Such dendrimer nanoparticles are not limited to particular uses. In certain embodiments, such dendrimer nanoparticles are used to sequester and/or identify Gram-positive and/or Gram-negative bacteria, screen liquid samples (e.g., water samples, food samples, pharmaceutical samples, blood samples, blood platelet samples, etc.) for the presence of Gram-positive and/or Gram-negative bacteria, treat disorders associated with Gram-positive and/or Gram-negative bacteria, and/or identify, sequester, and remove Gram-positive and/or Gram-negative bacteria from a liquid sample (e.g., water sample, food sample, pharmaceutical sample, blood sample, blood platelet sample, etc.). In certain embodiments, iron oxide nanoparticles are coated with such dendrimer nanoparticles.

In some embodiments, compositions comprising dendrimer nanoparticles conjugated with Vancomycin or Polymyxin (e.g., Polymyxin B, Polymyxin E) alone, or in combination, are used to treat disorders related to Gram-positive and Gram-negative bacteria and/or drug resistant forms of Gram-positive and Gram-negative bacteria (e.g., vancomycin-resistant Gram-positive bacteria) (e.g., polymyxin B or E resistant Gram-negative bacteria). In some embodiments, iron oxide nanoparticles are coated with such dendrimer nanoparticles conjugated with Vancomycin or Polymyxin (Polymyxin B, Polymyxin E) alone, or in combination. The methods are not limited to treating particular disorders related to Gram-positive and Gram-negative bacteria and/or drug resistant Gram-positive and Gram-negative bacteria. Examples include, but are not limited to, pneumonia, endocarditis, bacteremia, sepsis and other forms of toxemia caused by Gram-positive and Gram-negative bacteria. In some embodiments, a subject having or suspected of having a disorder related to Gram-positive and Gram-negative bacteria is administered a composition comprising dendrimer nanoparticles conjugated with Vancomycin or Polymyxin (e.g., Polymyxin B, Polymyxin E) alone, or in combination. In such embodiments, upon such administration, the dendrimer nanoparticles locate and bind with the Gram-positive and Gram-negative bacteria via the conjugated Vancomycin and/or Polymyxin (e.g., Polymyxin B, Polymyxin E), respectively, thereby ameliorating the effects of the Gram-positive and/or Gram-negative bacteria, and thereby treating the disorder. In some embodiments, additional agents are administered and/or co-administered with such compositions. Such agents include, but are not limited, to antibiotics (e.g., Gentamicin), and silver antibacterial agents.

In some embodiments, compositions comprising dendrimer nanoparticles conjugated with Vancomycin and imaging agents are used to identify Gram-positive bacteria within a sample. In some embodiments, compositions comprising dendrimer nanoparticles conjugated with two or more Vancomycin molecules (e.g., conjugated with an average of 2.3, 3.5 or 5.8 Vancomycin molecules) and imaging agents are used to identify Gram-positive bacteria within a sample. In some embodiments, compositions comprising dendrimer nanoparticles conjugated with Polymyxin (e.g., Polymyxin B, Polymyxin E) and imaging agents are used to identify Gram-negative bacteria within a sample. In some embodiments, compositions comprising dendrimer nanoparticles conjugated with two or more Polymyxin (e.g., Polymyxin B, Polymyxin E) molecules (e.g., conjugated with an average of 2.3, 3.5 or 5.8 Polymyxyin molecules) and imaging agents are used to identify Gram-negative bacteria within a sample. In some embodiments, compositions comprising dendrimer nanoparticles conjugated with Vancomycin, Polymyxin (e.g., Polymyxin B, Polymyxin E) and imaging agents are used to identify Gram-negative bacteria and Gram-negative bacteria within a sample. In some embodiments, compositions comprising dendrimer nanoparticles conjugated with two or more Vancomycin molecules (e.g., conjugated with an average of 2.3, 3.5 or 5.8 Vancomycin molecules), two or more Polymyxin (e.g., Polymyxin B, Polymyxin E) molecules (e.g., conjugated with an average of 2.3, 3.5 or 5.8 Polymyxyin molecules) and imaging agents are used to identify Gram-negative bacteria and Gram-negative bacteria within a sample. In some embodiments, iron oxide nanoparticles are coated with any of such dendrimer nanoparticles and imaging agents. In some embodiments, the Gram-positive and Gram-negative bacteria are drug-resistant Gram-positive bacteria (e.g., vancomycin-resistant Gram-positive bacteria) and drug resistant Gram-negative bacteria (e.g., polymyxin B or E resistant Gram-negative bacteria). In some embodiments, the sample is a liquid sample. In some embodiments, the liquid sample is, for example, a water sample, a food sample, a pharmaceutical sample, a blood sample (e.g., contaminated blood), or blood product samples (e.g., a blood platelet sample). Examples of imaging agents include, but are not limited to, molecular dyes, fluorescein isothiocyanate (FITC), 6-TAMRA, acridine orange, and cis-parinaric acid. In some embodiments, the imaging agents are molecular dyes from the alexa fluor (Molecular Probes) family of molecular dyes, Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), and Alexa Fluor 750 (red). Additional examples of imaging agents include, but are not limited to, MRI contrast agents, gadolinium-diethylenetriaminepentacetate (Gd-DTPA), and gadolium-1,4,7,10-tetraazacyclododecane-1,4,7,10-tetraacetic acid (Gd-DOTA). In some embodiments, the method comprises administering to the liquid sample a composition comprising such dendrimers, and wherein upon binding with such Gram-positive and/or Gram-negative bacteria, the imaging agents are detected. In some embodiments, the methods are used to detect the presence of Gram-positive and/or Gram-negative bacteria in a food product (e.g., to ensure the food sample is free of Gram-positive and/or Gram-negative bacteria). In some embodiments, the methods are used to detect the presence of Gram-positive and/or Gram-negative bacteria in a blood sample (e.g., a blood sample to be used in a blood transfusion) (e.g., to ensure the blood sample is free of Gram-positive and/or Gram-negative bacteria). In some embodiments, the methods are used to detect the presence of Gram-positive and/or Gram-negative bacteria in a blood product sample (e.g., blood platelets) (e.g., to ensure the blood product sample is free of Gram-positive and/or Gram-negative bacteria). In some embodiments, the methods are used to detect the presence of Gram-positive and/or Gram-negative bacteria in a water sample (e.g., to ensure the water sample is free of Gram-positive and/or Gram-negative bacteria). In some embodiments, the methods are used to detect the presence of Gram-positive and/or Gram-negative bacteria in a pharmaceutical sample (e.g., to ensure the pharmaceutical sample is free of Gram-positive and/or Gram-negative bacteria). In some embodiments, the concentration of Gram-positive and/or Gram-negative bacteria is detectable based upon the amount of imaging agent detected. In some embodiments, the methods are used to characterize the presence or absence of a Gram-positive and/or Gram-negative bacterial disorder.

In some embodiments, compositions comprising iron oxide nanoparticles coated with dendrimer nanoparticles conjugated with Vancomycin are used to sequester Gram-positive bacteria from a sample. In some embodiments, compositions comprising iron oxide nanoparticles coated with dendrimer nanoparticles conjugated with two or more Vancomycin molecules (e.g., conjugated with an average of 2.3, 3.5 or 5.8 Vancomycin molecules) are used to sequester Gram-positive bacteria from a sample. In some embodiments, compositions comprising iron oxide nanoparticles coated with dendrimer nanoparticles conjugated with Polymyxin (e.g., Polymyxin B, Polymyxin E) are used to sequester Gram-negative bacteria from a sample. In some embodiments, compositions comprising iron oxide nanoparticles coated with dendrimer nanoparticles conjugated with two or more Polymyxin (e.g., Polymyxin B, Polymyxin E) molecules (e.g., conjugated with an average of 2.3, 3.5 or 5.8 Polymyxyin molecules) are used to sequester Gram-negative bacteria from a sample. In some embodiments, the Gram-positive and Gram-negative bacteria are drug-resistant Gram-positive and Gram-negative bacteria. In some embodiments, the sample is a liquid sample. In some embodiments, the liquid sample is, for example, a water sample, a food sample, a pharmaceutical sample, a blood sample (e.g., contaminated blood), or blood product samples (e.g., a blood platelet sample). In some embodiments, the methods comprise administering to the liquid sample a composition comprising such iron oxide nanoparticles coated with such a dendrimer, and wherein upon binding with such Gram-positive and/or Gram-negative bacteria, a magnetic field and/or centrifugation is applied to the sample resulting in a sequestering of the Gram-positive and/or Gram-negative bacteria. In some embodiments, the methods comprise administering to the liquid sample a composition comprising such iron oxide nanoparticles coated with such a dendrimer (e.g., conjugated with Vancomycin and/or Polymyxin), and wherein upon binding with such Gram-positive and/or Gram-negative bacteria, a magnetic field and/or centrifugation is applied to the sample resulting in a sequestering of the Gram-positive and/or Gram-negative bacteria, and wherein the sequestered Gram-positive and/or Gram-negative bacteria are subsequently removed from the sample.

In some embodiments, the methods are used to detect for the presence of and/or screen for the presence of Gram-positive and/or Gram-negative bacteria in a food product (e.g., to screen the food sample for Gram-positive and/or Gram-negative bacterial contamination) (e.g., so as to alleviate a Gram-positive and/or Gram-negative bacteria contamination of the food sample). In some embodiments, the methods are used to detect for the presence of and/or screen for the presence of Gram-positive and/or Gram-negative bacteria in a blood sample (e.g., a blood sample to be used in a blood transfusion) (e.g., to screen the blood sample for Gram-positive and/or Gram-negative bacterial contamination) (e.g., so as to alleviate a Gram-positive and/or Gram-negative bacteria contamination of the blood sample). In some embodiments, the methods are used to detect for the presence of and/or screen for the presence of Gram-positive and/or Gram-negative bacteria in a blood product sample (e.g., blood platelets) (e.g., to screen the blood product sample for Gram-positive and/or Gram-negative bacterial contamination) (e.g., so as to alleviate a Gram-positive and/or Gram-negative bacteria contamination of the blood product sample). In some embodiments, the methods are used to detect for the presence of and/or screen for the presence of Gram-positive and/or Gram-negative bacteria in a water sample (e.g., to screen the water sample for Gram-positive and/or Gram-negative bacterial contamination) (e.g., so as to alleviate a Gram-positive and/or Gram-negative bacteria contamination of the water sample). In some embodiments, the methods are used to detect for the presence of and/or screen for the presence of Gram-positive and/or Gram-negative bacteria in a pharmaceutical sample (e.g., to screen the pharmaceutical sample for Gram-positive and/or Gram-negative bacterial contamination) (e.g., so as to alleviate a Gram-positive and/or Gram-negative bacteria contamination of the pharmaceutical sample).

Additional embodiments will be apparent to persons skilled in the relevant art based on the teachings contained herein.

### DESCRIPTION OF THE DRAWINGS

FIG. 1 shows (a) Schematic illustrating bacteria-targeting magnetic nanoparticles for the rapid pathogen isolation from blood products such as platelets (not shown to scale). (b and c) The success of this strategy is based, for example, on the multifunctionality of the iron oxide nanoparticles (IONP), each covered with multivalent dendrimer polymers conjugated with Vancomycin molecules, and/or Polymyxin molecules (e.g., Polymyxin B molecules, Polymyxin E molecules). Each of these antibiotic molecules serves a specific ligand for cell wall binding as its mode of antibiotic action. In addition, such multivalent presentation of the antibiotic ligands confers high avidity binding to the bacterial surface, and enable to adhere tightly to Gram-positive and/or Gram-negative bacterial cell.

FIG. 2 shows (A) Molecular mechanism for the recognition of the bacterial cell wall by the antibiotic vancomycin. The vancomycin molecule binds to the N^{α}-Ac-Lys-(D)-Ala-(D)-Ala terminus (X═NH; K_{D}≈10⁻⁶M) through five hydrogen bond (H bond) interactions, but such interactions are disrupted by the lactate (Lac; X═O; K_{D}≈10⁻³M)) exploited in the vancomycin-resistant bacterial cell wall which utilizes instead the N^{α}-Ac-Lys-(D)-Ala-(D)-Lac. (B, C) A schematic illustrating a multivalent strategy for tight binding to Gram-positive bacterial cells by using a fifth generation (G5) PAMAM dendrimer conjugated with multiple copies of vancomycin molecules. Such multivalent vancomycin conjugates confer high avidity binding to the bacterial surface, and enable them to target both vancomycin-susceptible and vancomycin-resistant bacterial cells. The size of the cell and the dendrimer particle are not drawn to scale.

FIG. 3 shows HPLC traces of vancomycin-conjugated dendrimers (Vancomycin)ₙ. (A) III-V Ac-G5-(V)ₙ (n=3.5, 5.8, 8.3); (B) Ac-G5-(V)ₙ (n=1.2, 2.3), VI GA-G5-(V)_{6.0} and VII DTPA-G5-(V)_{6.1}; (C) IX Ac-G5-(V)_{6.3}-(FITC)_{1.8} and VIII DTPA-G5-(V)_{6.1}-(Fl)_{3.9}. Note that structures and code names for these dendrimers are shown in Scheme 1.

FIG. 4 shows poissonian distribution of Ac-G5-(V)ₙ I-V, each having the mean valency of vancomycin at 1, 2, 4, or 6, respectively. The sum of populations (%) of multivalent species Ac-G5-(V)ₙ (n≧2) distributed in each conjugate I-IV is plotted in the inset.

FIG. 5 shows surface plasmon resonance (SPR) studies for the binding kinetics of vancomycin (A), and the vancomycin-presenting PAMAM dendrimers, IV Ac-G5-(V)_{5.8} (B) and VI GA-G5-(V)_{6.0} (C), to the vancomycin-susceptible bacterial cell wall model. The model is made by immobilization of N^{α}-Ac-Lys-(D)-Ala-(D)-Ala peptide molecules on the CMS sensor chip. The concentrations of vancomycin and its dendrimer conjugates injected are indicated in the overlay of the sensorgrams. The inset (A) is the Scatchard plot derived from the SPR data, and used to determine the dissociation constant (K_{D}) of vancomycin.

FIG. 6 shows surface plasmon resonance (SPR) sensorgrams of Ac-G5-(V)ₙ. The conjugate IV, Ac-G5-(V)_{5.8}, was flowed in to a bacterial model surface (A) that presents N^{α}-Ac-Lys-(D)-Ala-(D)-Ala peptide molecules (flow cell 1), and to a reference surface (B) that presents no such peptides (flow cell 2). (C) Corrected sensorgrams of IV Ac-G5-(V)_{5.8}, each obtained by subtraction of the reference sensorgram: ΔRU=RU₁ (flow cell 1)−Ru₂ (flow cell 2). (D) Corrected sensorgrams for the binding of II Ac-G5-(V)_{2.3} to the (D)-Ala-(D)-Ala surface. (E) Corrected sensorgrams for a fully acetylated dendrimer (Ac-G5)—a negative control dendrimer—to the (D)-Ala-(D)-Ala surface.

FIG. 7 shows surface plasmon resonance (SPR) studies for the binding kinetics of vancomycin, and the vancomycin-presenting PAMAM dendrimers G5-(V)ₙ to the vancomycin-resistant bacterial cell wall model. The model is made by immobilization of N^{α}-Ac-Lys-(D)-Ala-(D)-Lac peptide molecules on the CMS sensor chip. SPR sensorgrams for vancomycin (A), IV Ac-G5-(V)_{5.8} (B), and VI GA-G5-(V)_{6.0} (C) are acquired at the range of the concentrations as indicated. The inset (A) is the Scatchard plot derived from the SPR data in order to determine the K_{D} value of vancomycin. Fitting curves are illustrated for those sensorgrams (B) as overlaid in black lines.

FIG. 8 shows surface plasmon resonance (SPR) sensorgrams for the binding of vancomycin-presenting PAMAM dendrimers G5-(V)ₙ to a vancomycin-resistant bacterial cell wall model which presents N^{α}-Ac-Lys-(D)-Ala-(D)-Lac peptide molecules on the sensor chip surface. SPR sensorgrams for I Ac-G5-(V)_{1.2} (A), II Ac-G5-(V)_{2.3} (B), III Ac-G5-(V)_{3.5} (C) and VII DTPA-G5-(V)_{6.1} (D) are acquired at a series of concentrations as indicated. Fitting curves for those sensorgrams in A and C are overlaid in black lines. Each sensorgram is corrected as noted earlier: ΔRU=RU₁ (the (D)-Ala-(D)-Lac surface; flow cell 1)−RU₂ (reference surface; flow cell 2).

FIG. 9 shows (A) An array of selected SPR sensorgrams for Ac-G5-(V)ₙ binding to (D)-Ala-(D)-Lac peptide molecules on the surface, each acquired at the identical concentration including I Ac-G5-(V)_{1.2} (50 nM), II Ac-G5-(V)_{2.3} (50 nM), III Ac-G5-(V)_{3.5} (51 nM), and IV Ac-G5-(V)_{5.8} (50 nM). (B) Relative adsorption (RU_{A}) of Ac-G5-(V)ₙ and fraction (%) of multivalent populations (n≧2; inset of FIG. 2). Relative adsorption is defined as RU_{A} (conjugate) relative to RU_{A} (IV; 100%). (C) Comparison of off-rate constant k_{off} (Table 2) and fractional desorption of Ac-G5-(V)ₙ as a function of valency (n). The fractional desorption is defined as the level of the dendrimer desorbed (RU_{D}) relative to the level of the dendrimer adsorbed (RU_{A}) as illustrated for the conjugate IV. Each value of the RU_{A} and RU_{D} was calculated as the mean value from at least six different injection concentrations per conjugate at the specific time point indicated. Each error bar indicates the standard error of the mean (SEM).

FIG. 10 shows equilibrium dissociation constants (K_{D}, M) of G5-(V)ₙ I-IV, VI determined by the SPR binding to the cell wall model made of either (D)-Ala-(D)-Ala or (D)-Ala-(D)-Lac peptides as a function of valency (n). The K_{D} values for free vancomycin are placed arbitrarily around n=0.9 as the reference point indicative of the affinity constant for its monovalent association. Each error bar indicates the standard error of the mean (SEM).

FIG. 11 shows confocal images of Gram-positive bacterial cells (Staphylococcus aureus: ATCC 4012) treated with VIII DTPA-G5-(V)_{6.1}-(Fl)_{3.9} (A), IX Ac-G5-(V)_{6.3}-(FITC)_{1.8} (B), or GA-GS-(FITC) as a non-targeted control dendrimer (C). The cells were incubated with each of the dendrimers (86 μM), washed, and stained with Syto® 59, a cell-permeable fluorescent molecule (emission wavelength=645 nm; red) that intercalates into the DNA molecules inside the nucleus. Adsorption of the dendrimer nanoparticles to the bacterial cells is indicated by the green fluorescence (Fluorescein; emission wavelength=520 nm) in those cells treated with VIII or IX, but not by the negative control GA-G5-(FITC). The fluorescent objects in (B) show much larger images than as expected for individual bacterial cells and are attributable to the bacterial aggregates, possibly due to the crosslinking by the vancomycin-conjugated multivalent dendrimers (scale bar=20 μm).

FIG. 12 shows a turbidity assay to determine the ability to cause cell lysis by vancomycin-conjugated dendrimers VIII DTPA-G5-(V)_{6.1}-(Fl)_{3.9} and IX Ac-G5-(V)_{6.3}-(FITC)_{1.8} against Gram-positive bacterial cells (Staphylococcus aureus). Test concentrations for each conjugate are given on the molar basis of the vancomycin molecules in the solution, [Vancomycin]=n×[G5−(V)ₙ].

FIG. 13 shows (A) turbidity assay for vancomycin-conjugated dendrimers, I-VI G5-(V)ₙ, against Gram-positive bacterial cells (Staphylococcus aureus; ATCC 4012). Test concentrations for each dendrimer-vancomycin conjugate are given on a molar basis of vancomycin: [Vancomycin]=n×[G5−(V)ₙ]; (B, C) In vitro cytotoxicity of vancmoycin-conjugated dendrimers, each tested in human cervical carcinoma KB cell line (B), and in mouse melanoma B16-F10 cells (C). G5-NH₂ (unmodified G5 PAMAM dendrimer), II Ac-G5-(V)_{2.3}, IV Ac-G5-(V)_{5.8}, VI GA-G5-(V)_{6.0}, VII DTPA-G5-(V)_{6.1}.

FIG. 14 shows magnetic isolation of Gram-positive bacterial cells (Staphylococcus aureus) by using cell wall-targeting magnetic nanoparticles IONP-VI and IONP-VII. Synthesis of these IONPs is described in Scheme 2. Variable titers of bacterial cells were incubated with either IONP-VI, IONP-VII, or a control IONP (no dendrimer coated), each at 0.25 IONP mg per 1.0×10⁸ CFU bacteria (A), and 2.0×10⁴-3.0×10⁶ CFU bacteria (B). Those bound with magnetic nanoparticles were isolated under the magnetic field. The level of the bacterial cells isolated (I) or retained in the supernatant (S) after each treatment was quantified by the cell culture assay, and expressed as % colony forming unit (CFU) relative to the control level (C; no treatment). The results are presented as mean±standard error of the mean (SEM; 12-17%).

FIG. 15 shows gel permeation chromatography (GPC) chromatograms of G5 PAMAM dendrimer conjugates, each linked with vancomycin (V) molecules at a variable ratio (n) of vancomycin to the dendrimer molecule. (A) I-V Ac-G5-(V)ₙ (n=1.2, 2.3, 3.5, 5.8, 8.3); (B) VII DTPA-G5-(V)_{6.1}.

FIG. 16 shows UV-vis spectra of G5 dendrimer-vancomycin conjugates G5-(V)ₙ. Each of the dendrimer conjugates was measured in PBS (pH 7.4) at the concentration of the dendrimer as indicated. (A) III Ac-G5-(V)_{3.5} ([dendrimer]=7.0 μM), IV Ac-G5-(V)_{5.8} (6.5 μM), V Ac-G5-(V)_{8.3} (5.8 μM); (B) II Ac-G5-(V)_{2.3} (7.1 μM), VI GA-G5-(V)_{6.0} (5.2 μM), I Ac-G5-(V)_{1.2} (7.6 μM); VII DTPA-G5-(V)_{6.1} (4.2 μM); (C) VIII DTPA-G5-(V)_{6.1}-(Fl)_{3.9} (6.5 μM), IX Ac-G5-(V)_{6.3}-(FITC)_{1.8} (6.4 μM.)

FIG. 17 shows selected ¹H NMR spectra of vancomycin-conjugated dendrimers G5-(V)ₙ. (A) II Ac-G5-(V)_{2.3}; (B) III Ac-G5-(V)_{3.5}; (C) IV Ac-G5-(V)_{5.8}, (D) VI GA-G5-(V)_{6.0}. Each NMR spectrum was acquired in D₂O (5 mg/mL).

FIG. 18 shows MALDI TOF mass spectra of vancomycin (V)-conjugated G5 PAMAM dendrimers, I-V Ac-G5-(V)ₙ (n=1.2, 2.3, 3.5, 5.8, 8.3) and VI GA-G5-(V)_{6.0}.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “Gram-positive bacteria” includes those bacteria that are stained dark blue or violet by Gram staining such as Bacillus, Listeria, Staphylococcus, Enterococcus, Lactobacillus, Lactococcus, Leuconostoc, Pectinatus, Pediococcusm, Streptococcus, Acetobacterium, Clostridium, Eubacterium, Heliobacterium, Heliospirillum, Sporomusa, and Actinobacteria. The Gram-positive bacteria also includes bacteria that lack cell walls and so cannot be stained by Gram but are nonetheless related to bacteria that can be stained by Gram. Examples of these include but are not limited to Mycoplasma, Spiroplasma, Ureaplasma, and Erysipelothrix. Generally, Gram-positive bacteria have (D)-Ala-(D)-Ala residue present on the bacterial surface (e.g., a (D)-Ala-(D)-Ala cell wall precursor) (e.g., a (D)-Ala-(D)-Ala on the proteoglycan layer).

As used herein, the term “Gram-negative bacteria” includes those bacteria that are not stained dark blue or violet by Gram staining such as Escherichia coli (E. coli), Salmonella, Shigella, Pseudomonas, Helicobacter, Haemophilus, Actinobacillus, Burkholderia mallei and Francisella tularensis. Generally, Gram-negative bacteria have lipopolysaccharide residues present on the bacterial surface as a cell wall component.

As used herein, the term “drug-resistant Gram-positive bacteria,” “Gram-negative bacteria,” or “drug-resistant Gram-positive and/or Gram-negative bacteria” includes bacteria that is resistant to traditional antibiotic treatment (e.g., Vancomycin and/or Polymyxin (e.g., Polymyxin B, Polymyxin E) treatment). In particular, drug-resistant Gram-positive bacteria have (D)-Ala-(D)-Lac residue present on the bacterial surface (e.g., a (D)-Ala-(D)-Lac cell wall precursor) (e.g., a (D)-Ala-(D)-Lac on the proteoglycan layer). In particular, drug-resistant Gram-negative bacteria have (D)-Ala-(D)-Lac residue present on the bacterial surface (e.g., a (D)-Ala-(D)-Lac cell wall precursor) (e.g., a (D)-Ala-(D)-Lac on the proteoglycan layer).

As used herein, the term “Vancomycin” refers to a glycopeptide antibiotic used, for example, in the prophylaxis and treatment of infections caused by Gram-positive bacteria. The term “Vancomycin” includes, but is not limited to, Vancomycin molecules and equivalents thereof.

As used herein, the term “Polymyxin” refers to an antibiotic with a general structure consisting of a cyclic peptide with a long hydrophobic tail. Polymyxin is known to disrupt the structure of Gram-negative bacterial cell membranes by interacting with its phospholipids. Polymyxin is selectively toxic for Gram-negative bacteria due to its specificity for the lipopolysaccharide molecule that exists within many Gram-negative outer membranes. Examples of Polymyxin include, but are not limited to, Polymyxin B (and equivalents thereof), and Polymyxin E (and equivalents thereof). As used herein, the term “subject suspected of having a Gram-positive and/or Gram-negative bacterial disorder” refers to a subject that presents one or more symptoms indicative of a Gram-positive and/or Gram-negative bacterial disorder (e.g., pneumonia, endocarditis, bacteremia, sepsis and other forms of toxemia) or is being screened for a Gram-positive and/or Gram-negative bacterial disorder. As used herein, the term “subject diagnosed with a Gram-positive and/or Gram-negative bacterial disorder” refers to a subject who has been tested and found to have a Gram-positive and/or Gram-negative bacterial disorder (e.g., pneumonia, endocarditis, bacteremia, sepsis and other forms of toxemia).

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention. Liquid samples generally pertain to water samples, food samples, blood samples, blood product samples, and pharmaceutical samples.

As used herein, the term “drug” is meant to include any molecule, molecular complex or substance administered to an organism for diagnostic or therapeutic purposes, including medical imaging, monitoring, contraceptive, cosmetic, nutraceutical, pharmaceutical and prophylactic applications. The term “drug” is further meant to include any such molecule, molecular complex or substance that is chemically modified and/or operatively attached to a biologic or biocompatible structure.

As used herein, the term “purified” or “to purify” or “compositional purity” refers to the removal of components (e.g., contaminants) from a sample or the level of components (e.g., contaminants) within a sample. For example, unreacted moieties, degradation products, excess reactants, or byproducts are removed from a sample following a synthesis reaction or preparative method.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using screening methods known in the art.

The term “monovalency” refers to a single binding interaction between one ligand molecule (e.g., Vancomycin, Polymyxin (e.g., Polymyxin B, Polymyxin E) with one ligand binding site on the bacterial surface (e.g., D-Ala-D-Ala peptide residue for Vancomycin; lipopolysaccharide for Polymyxin (e.g., Polymyxin B, Polymyxin E)).

The term “multivalency” refers to the concurrent binding of multiple ligands, which may be the same or different, with multiple corresponding ligand binding sites.

The term “multivalent nanoparticle” refers to a single entity (e.g., dendrimer) that presents multiple (≧2) number of ligand molecules covalently attached on the nartiple. For example, multivalent dendrimer nanoparticles are conjugated with two or more Vancomycin molecules and/or Polymyxin B or E molecules (e.g., conjugated with an average of 2.3, 3.5 or 5.8 Vancomycin and/or Polymyxin molecules).

As used herein, the term “nanodevice” or “nanodevices” refer, generally, to compositions comprising dendrimers of the present invention. As such, a nanodevice may refer to a composition comprising a dendrimer of the present invention that may contain one or more ligands, linkers, and/or functional groups (e.g., a therapeutic agent, a targeting agent, a trigger agent, an imaging agent) conjugated to the dendrimer (e.g., a dendrimer nanoparticle conjugated with Vancomycin molecules and/or Polymyxin (e.g., Polymyxin B, Polymyxin E) molecules).

As used herein, the term “degradable linkage,” when used in reference to a polymer refers to a conjugate that comprises a physiologically cleavable linkage (e.g., a linkage that can be hydrolyzed (e.g., in vivo) or otherwise reversed (e.g., via enzymatic cleavage). Such physiologically cleavable linkages include, but are not limited to, ester, carbonate ester, carbamate, sulfate, phosphate, acyloxyalkyl ether, acetal, and ketal linkages (See, e.g., U.S. Pat. No. 6,838,076, herein incorporated by reference in its entirety). Similarly, the conjugate may comprise a cleavable linkage present in the linkage between the dendrimer and functional group, or, may comprise a cleavable linkage present in the polymer itself (See, e.g., U.S. Pat. App. Nos. 20050158273 and 20050181449, each of which is herein incorporated by reference in its entirety).

As used herein, the term “Baker-Huang dendrimer” or “Baker-Huang PAMAM dendrimer” refers to a dendrimer comprised of branching units of structure:

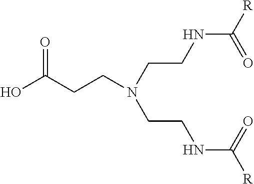

wherein R comprises a carbon-containing functional group (e.g., CF₃). In some embodiments, the branching unit is activated to its NHS ester. In some embodiments, such activation is achieved using TSTU. In some embodiments, EDA is added. In some embodiments, the dendrimer is further treated to replace, e.g., CF₃ functional groups with NH₂ functional groups; for example, in some embodiments, a CF₃-containing version of the dendrimer is treated with K₂CO₃ to yield a dendrimer with terminal NH₂ groups (for example, as shown in Scheme 2). In some embodiments, terminal groups of a Baker-Huang dendrimer are further derivatized and/or further conjugated with other moieties. For example, one or more functional ligands (e.g., for therapeutic, targeting, imaging, or drug delivery function(s)) may be conjugated to a Baker-Huang dendrimer, either via direct conjugation to terminal branches or indirectly (e.g., through linkers, through other functional groups (e.g., through an OH-functional group)). In some embodiments, the order of iterative repeats from core to surface is amide bonds first, followed by tertiary amines, with ethylene groups intervening between the amide bond and tertiary amines In preferred embodiments, a Baker-Huang dendrimer is synthesized by convergent synthesis methods. As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterized. For example, an inhibitor can be selected from the group comprising, but not limited to, those found in U.S. Pat. No. 6,011,021, herein incorporated by reference in its entirety.

A “physiologically cleavable” or “hydrolysable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that is subject to degradation by one or more enzymes.

A “hydrolytically stable” linkage or bond refers to a chemical bond (e.g., typically a covalent bond) that is substantially stable in water (i.e., does not undergo hydrolysis under physiological conditions to any appreciable extent over an extended period of time). Examples of hydrolytically stable linkages include, but are not limited to, carbon-carbon bonds (e.g., in aliphatic chains), ethers, amides, urethanes, and the like.

As used herein, the term “click chemistry” refers to chemistry tailored to generate substances quickly and reliably by joining small modular units together (see, e.g., Kolb et al. (2001) Angewandte Chemie Intl. Ed. 40:2004-2011; Evans (2007) Australian J. Chem. 60:384-395; Carlmark et al. (2009) Chem. Soc. Rev. 38:352-362; each herein incorporated by reference in its entirety).

As used herein, the term “triazine” refers to a compound comprising a ring structure bearing three nitrogen atoms. In some embodiments, the ring structure is six-membered (e.g., the molecular formula comprises C₃H₃N₃). In some embodiments, the ring is a conjugated system. Triazine moieties with six-membered rings may have nitrogen atoms at any possible placement so long as three nitrogen atoms occur in the ring (e.g., 1,2,3-triazine; 1,2,4-triazine, 1,3,5-triazine, 1,2,5-triazine, 1,2,6-triazine, etc.)

As used herein, the term “scaffold” refers to a compound to which other moieties are attached (e.g., conjugated). In some embodiments, a scaffold is conjugated to bioactive functional conjugates (e.g., a therapeutic agent, a targeting agent, a trigger agent, an imaging agent). In some embodiments, a scaffold is conjugated to a dendrimer (e.g., a PAMAM dendrimer). In some embodiments, conjugation of a scaffold to a dendrimer and/or a functional conjugate(s) is direct, while in other embodiments conjugation of a scaffold to a dendrimer and/or a functional conjugate(s) is indirect, e.g., an intervening linker is present between the scaffold compound and the dendrimer, and/or the scaffold and the functional conjugate(s).

As used herein, the term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. As used herein, an “ester coupling agent” refers to a reagent that can facilitate the formation of an ester bond between two reactants. The present invention is not limited to any particular coupling agent or agents. Examples of coupling agents include but are not limited to 2-chloro-1-methylpyridium iodide and 4-(dimethylamino) pyridine, or dicyclohexylcarbodiimide and 4-(dimethylamino) pyridine or diethyl azodicarboxylate and triphenylphosphine or other carbodiimide coupling agent and 4-(dimethylamino)pyridine.

As used herein, the term “glycidolate” refers to the addition of a 2,3-dihydroxylpropyl group to a reagent using glycidol as a reactant. In some embodiments, the reagent to which the 2,3-dihydroxylpropyl groups are added is a dendrimer. In some embodiments, the dendrimer is a PAMAM dendrimer. Glycidolation may be used generally to add terminal hydroxyl functional groups to a reagent.

As used herein, the term “ligand” refers to any moiety covalently attached (e.g., conjugated) to a dendrimer branch; in preferred embodiments, such conjugation is indirect (e.g., an intervening moiety exists between the dendrimer branch and the ligand) rather than direct (e.g., no intervening moiety exists between the dendrimer branch and the ligand). Indirect attachment of a ligand to a dendrimer may exist where a scaffold compound (e.g., triazine scaffold) intervenes. In preferred embodiments, ligands have functional utility for specific applications, e.g., for therapeutic, targeting, imaging, or drug delivery function(s). The terms “ligand”, “conjugate”, and “functional group” may be used interchangeably.

### DETAILED DESCRIPTION OF THE INVENTION

Bacterial infections cause many serious medical conditions including sepsis, bacteremia, pneumonia and endocarditis (see, e.g., Jacoby, G. A.; et al., N. Engl. J. Med. 1991, 324, 601-12; Dajani, A. S.; Circulation 1997, 96, 358-66; Tipple, M. A.; et al., Transfusion (Malden, Mass., U.S.) 1990, 30, 207-13; 4. Muder, R. R.; Yee, Y. C.; Rihs, J. D.; et al., Transfusion (Malden, Mass., U.S.) 1992, 32, 771-74; each herein incorporated by reference in its entirety)). These infections can be life-threatening, especially when associated with drug-resistant bacteria (see, e.g., Jacoby, G. A.; et al., N. Engl. J. Med. 1991, 324, 601-12; Cosgrove, S. E.; et al., Clin. Infect. Dis. 2003, 36, 53-59; each herein incorporated by reference in its entirety)). Bacterial contamination of whole blood or blood-derived products is a long-standing, unsolved problem in transfusion medicine (see, e.g., Wagner, S. J.; et al., Clinical Microbiology Reviews 1994, 7, 290-302; McKane, A. V.; et al., American Journal of Clinical Pathology 2009, 131, 542-51; Depcik-Smith, N. D.; et al., Journal of Clinical Apheresis 2001, 16, 192-201; Bihl, F.; et al., Journal of Translational Medicine 2007, 5, 25; each herein incorporated by reference in its entirety)). In particular, whole blood-derived platelets present a greater risk of transfusion-transmitted fatal bacterial infections due to their higher rates of bacterial contaminations that occur during storage (see, e.g., Pearce, S.; et al., Transfusion Medicine 2011, 21, 25-32; herein incorporated by reference in its entirety)). In transfusion clinics, platelets are routinely screened for bacterial contamination prior to transfusion, and current lab tests performed for this purpose are disappointing in the lower limit of bacterial detection by showing ≧10³-10⁴ CFU. Such detection sensitivity is far from preventing bacterial infections. A significant factor that contributes to this poor sensitivity is lack of sufficient test time since only one hour or so is typically available for the platelet screening. Therefore, there remains an urgent unmet need for developing a practical method for screening platelets that provides better detection sensitivity within a short timeframe (see, e.g., Schmidt, M.; et al., Transfusion Medicine and Hemotherapy 2011, 38, 259-65; Goodnough, L. T.; et al., The Lancet 2003, 361, 161-69; each herein incorporated by reference in its entirety)).

Some assay methods validated for bacterial detection are acceptable for sensitivity that include the microbial cell culture, PCR (see, e.g., Dreier, J.; et al., Transfusion Medicine Reviews 2007, 21, 237-54; herein incorporated by reference in its entirety)), FACS (see, e.g., Schmidt, M.; et al., Transfusion Medicine 2006, 16, 355-61; herein incorporated by reference in its entirety)), and ELISA (see, e.g., Fleming, P.; Transfusion 2008, 48, 1-1; herein incorporated by reference in its entirety)). However most of the assays normally require longer assay times to achieve higher sensitivity as illustrated by the culture assay that takes 3-5 days or longer despite its capability to detect bacteria as low as ≧1 colony-forming unit (CFU)/mL blood (see, e.g., Schmidt, M.; et al., Transfusion Medicine 2006, 16, 355-61; herein incorporated by reference in its entirety)). Such assay format is not applicable for screening platelets which needs to be completed within only a few hours before transfusion begins. At present, four FDA approved assays are available in the transfusion service clinics for detection of bacterial contamination in platelet components. Two of these methods (Biomerieux BacT/Alert™ and Haemonetics eBDS™) are culture based and are primarily used at the time of component manufacture. Two other methods (Verax PGD™ and Immunetics BacTx™) are intended for use prior to component release. However they are less sensitive than culture (≧10³-10⁴ CFU/mL) but can be implemented at the level of the transfusion service.

Gram-positive and Gram-negative bacterial infections (see, e.g., Bihl, F.; et al., J. Transl. Med. 2007, 5, 25; Depcik-Smith, N. D.; et al., J. Clinical Apheresis 2001, 16, 192-201; each herein incorporated by reference in its entirety)) cause numerous serious medical conditions including sepsis, bacteremia, pneumonia and endocarditis (see, e.g., Jacoby, G. A.; et al., N. Engl. J. Med. 1991, 324, 601-612; Dajani, A. S.; et al., Circulation 1997, 96, 358-366; Tipple, M. A.; et al., Transfusion (Malden, Mass., U.S.) 1990, 30, 207-213; Muder, R. R.; et al., Transfusion (Malden, Mass., U.S.) 1992, 32, 771-774; each herein incorporated by reference in its entirety)). Such infections can be life-threatening, especially when associated with drug-resistant bacteria (see, e.g., Jacoby, G. A.; et al., N. Engl. J. Med. 1991, 324, 601-612; Cosgrove, S. E.; et al., Clin. Infect. Dis. 2003, 36, 53-59; each herein incorporated by reference in its entirety)). Despite the fact that there are growing concerns about serious infectious diseases, current technology is not able to fully and accurately detect, enumerate, or treat those causative bacterial cells inhabiting the blood and other vital organs (see, e.g., Schmidt, M.; et al., Transfusion Medicine and Hemotherapy 2011, 38, 259-265; herein incorporated by reference in its entirety)).

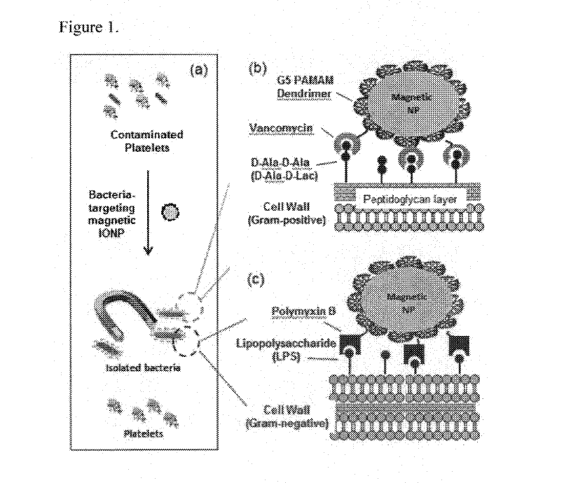

The present invention provides a novel nanotechnology that supports the right combination of higher sensitivity, speed and ease of the assay for detecting bacteria in aqueous samples (see, e.g., FIG. 1). Indeed, experiments conducted during the course of developing embodiments for the present invention involved a biophysical evaluation and practical exploration of vancomycin-presenting, poly(amidoamine) (PAMAM) dendrimers as a platform enabling detection and isolation of bacterial pathogens.

Accordingly, the present invention provides a novel nanotechnology that provides a combination of higher sensitivity, speed and ease of the assay for detecting bacteria in aqueous samples (see, e.g., FIG. 1). Indeed, embodiments of the present invention represent a significant technological advance, with the inherent modularity of the nanoparticle technology providing many options for rapid, sensitive, and portable screening of bacterial contaminants from blood and blood products. The clinical significance of this work is considerable, as a significant outcome is a reduction in the number of adverse events suffered by patients as a result of transfusion-related sepsis. Indeed, preventative interventions that are so critical to transfusion medicine are enhanced by the technology that monitors bacterial contamination of platelets.

Recently, rapid advances have been made in cell-targeted delivery systems that cover a wide range of therapeutic areas from cancers and inflammatory diseases to infections (see, e.g., Kell, A. J.; et al., ACS Nano 2008, 2, 1777-1788; Kukowska-Latallo, J. F.; et al., Cancer Res. 2005, 65, 5317-5324; Low, P. S.; et al., Acc. Chem. Res. 2008, 41, 120-129; Myc, A.; et al., Biomacromolecules 2007, 8, 13-18; Thomas, T. P.; et al., Arthritis Rheum. 2011, 63, 2671-2680; each herein incorporated by reference in its entirety)). Such delivery platforms are typically composed of a nanometer-sized particle (NP) or scaffold conjugated with high affinity small molecule ligands or antibodies to bind to specific surface biomarkers (see, e.g., Low, P. S.; et al., Acc. Chem. Res. 2008, 41, 120-129; Majoros, I. J.; et al., WIREs: Nanomed. Nanobiotech. 2009, 1, 502-510; each herein incorporated by reference in its entirety)). This targeting strategy allows cell-specific delivery of payloads such as small molecule chemotherapeutics, therapeutic genes, and imaging molecules also carried by the nanoparticles (see, e.g., Majoros, I. J.; et al., WIREs: Nanomed. Nanobiotech. 2009, 1, 502-510; Kukowska-Latallo, J. F.; et al., Cancer Res. 2005, 65, 5317-5324; Choi, S. K.; et al., Chem. Commun. (Cambridge, U.K.) 2010, 46, 2632-2634; each herein incorporated by reference in its entirety)).

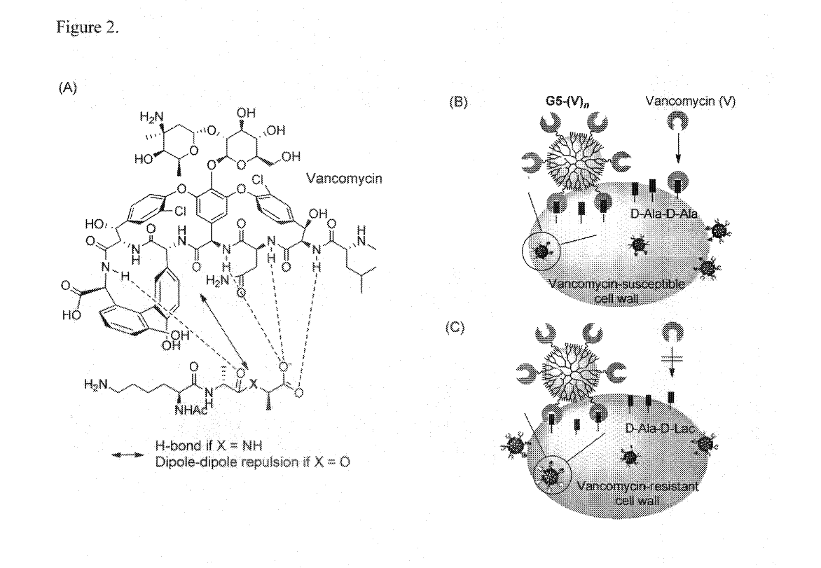

As a micron-sized organism, the bacterium expresses a high density of various surface molecules on its cell wall that serve as rich opportunities for selective recognition of the cell. In fact, the modes of action associated with standard antimicrobial agents are attributable to binding and destabilization of the cell wall structure as seen with vancomycin (see, e.g., Walsh, C.; Nature (London, U.K.) 2000, 406, 775-781; herein incorporated by reference in its entirety)), beta-lactams (see, e.g., Tipper, D. J.; Pharmacol. Ther. 1985, 27, 1-35; herein incorporated by reference in its entirety)), and polymyxins (see, e.g., Velkov, T.; et al., J. Med. Chem. 2009, 53, 1898-1916; herein incorporated by reference in its entirety)). Of these agents, vancomycin has been investigated as a molecular probe for targeting Gram-positive bacteria because of its strong affinity to a cell wall precursor terminated with a (D)-Ala-(D)-Ala peptide residue (Ala=alanine; FIG. 2; KD≈10−6M) (see, e.g., Kell, A. J.; et al., ACS Nano 2008, 2, 1777-1788; Chung, H. J.; et al., ACS Nano 2011, 5, 8834-8841; Krishnamurthy, V. M.; et al., Biomaterials 2006, 27, 3663-3674; Metallo, S. J.; et al., J. Am. Chem. Soc. 2003, 125, 4534-4540; each herein incorporated by reference in its entirety)). Polymyxin class of antibiotics (e.g., Polymyxin B, Polymyxin E) target a Gram-negative organism due to their strong ability to bind anionic lipopolysaccharides exposed from the outer membrane (see, FIG. 1; K_{D}≈10⁻⁶ M) (see, e.g., Moore, R. A.; et al., Antimicrobial Agents and Chemotherapy 1986, 29, 496-500; Thomas, C. J.; et al., J. Biol. Chem. 1999, 274, 29624-27; each herein incorporated by reference in its entirety). Additional studies reported on the practical applications of multivalent vancomycin molecules (see, e.g., Rao, J.; et al., Science (WAshington, D.C., U.S.) 1998, 280, 708-711; Rao, J.; et al., J. Am. Chem. Soc. 1997, 119, 10286-10290; Rao, J.; et al., Chem. Biol. (Cambridge, Mass., U.S.) 1999, 6, 353-359; Rao, J.; et al., J. Am. Chem. Soc. 1999, 121, 2629-2630; Xing, B.; et al., J. Med. Chem. 2003, 46, 4904-4909; each herein incorporated by reference in its entirety)) as well as vancomycin-conjugated NPs which are made on the scaffold of poly(acrylamide) polymers (see, e.g., Krishnamurthy, V. M.; et al., Biomaterials 2006, 27, 3663-3674; Metallo, S. J.; et al., J. Am. Chem. Soc. 2003, 125, 4534-4540; each herein incorporated by reference in its entirety)), and by direct attachment to the surface of inorganic NPs of gold (see, e.g., Gu, H.; et al., Nano Lett. 2003, 3, 1261-1263; herein incorporated by reference in its entirety)) and iron oxide (see, e.g., Kell, A. J.; et al., ACS Nano 2008, 2, 1777-1788; Gu, H.; et al., J. Am. Chem. Soc. 2003, 125, 15702-15703; each herein incorporated by reference in its entirety)). These studies demonstrated the effectiveness of such vancomycin conjugates in inducing bacteria-targeted opsonization activity (see, e.g., Krishnamurthy, V. M.; et al., Biomaterials 2006, 27, 3663-3674; Metallo, S. J.; et al., J. Am. Chem. Soc. 2003, 125, 4534-4540; each herein incorporated by reference in its entirety)), enhancing antimicrobial activity (see, e.g., Gu, H.; et al., Nano Lett. 2003, 3, 1261-1263; herein incorporated by reference in its entirety)), and enabling the concentration of bacterial cells (see, e.g., Kell, A. J.; et al., ACS Nano 2008, 2, 1777-1788; Gu, H.; et al., J. Am. Chem. Soc. 2003, 125, 15702-15703; each herein incorporated by reference in its entirety)).

Despite such strong potential demonstrated for cell wall-associated applications, vancomycin is not active against vancomycin-resistant enterococci (VRE) because it has weak affinity to the (D)-Ala-(D)-Lac residue (Lac=lactate; K_{D}≈10⁻³M) present on the bacterial surface of this species, resulting in vancomycin resistance (see, e.g., Walsh, C.; Nature (London, U.K.) 2000, 406, 775-781; Walsh, C. T.; et al., Chem. Biol. (Cambridge, Mass., U.S.) 1996, 3, 21-28; each herein incorporated by reference in its entirety)). Experiments conducted during the course of developing embodiments for the present invention applied a multivalent ligand design (see, e.g., Griffin, J. H.; et al., J. Am. Chem. Soc. 2003, 125, 6517-6531; Hong, S.; et al., Chem. Biol. (Cambridge, Mass., U.S.) 2007, 14, 107-115; Mammen, M.; et al., Angew. Chem. Int. Ed. 1998, 37, 2754-2794; Roy, R.; Curr. Opin. Struct. Biol. 1996, 6, 692-702; each herein incorporated by reference in its entirety)) for vancomycin and tested the hypothetical notion that the suboptimal affinity of vancomycin could be enhanced by the use of a multivalent dendrimer. Defined as a molecular construct that presents multiple copies of an identical ligand tethered to a scaffold (see, e.g., Mammen, M.; et al., Angew. Chem. Int. Ed. 1998, 37, 2754-2794; Roy, R.; Curr. Opin. Struct. Biol. 1996, 6, 692-702; Lee, Y. C.; et al., Acc. Chem. Res. 1995, 28, 321-327; Kiessling, L. L.; et al., Curr. Opin. Chem. Biol. 2000, 4, 696-703; each herein incorporated by reference in its entirety)), the multivalent molecule was shown to bind simultaneously to multiple receptors on the biological surface and, as a consequence, displayed collectively much tighter binding avidity than the affinity displayed by each monovalent ligand attached.

In certain embodiments, the present invention provides fifth generation (G5) PAMAM dendrimers as the scaffold for the multivalent vancomycin design. As a synthetic polymer NP (diameter 5.4 nm) (see, e.g., Tomalia, D. A.; et al., Polymer J. 1985, 17, 117-132; Tomalia, D. A.; et al., Angew. Chem., Int. Ed. Engl. 1990, 29, 138-175; each herein incorporated by reference in its entirety)), the G5 dendrimer has been extensively investigated for use in applications in targeted drug delivery (see, e.g., Kukowska-Latallo, J. F.; et al., Cancer Res. 2005, 65, 5317-5324; Thomas, T. P.; et al., Arthritis Rheum. 2011, 63, 2671-2680; Choi, S. K.; et al., Chem. Commun (Cambridge, U.K.) 2010, 46, 2632-2634; Hong, S.; et al., Chem. Biol. (Cambridge, Mass., U.S.) 2007, 14, 107-115; Majoros, I.; Baker Jr, J. Dendrimer-Based Nanomedicine. Pan Stanford: Hackensack, N.J., 2008; p 436; Thomas, T. P.; et al., Bioorg. Med. Chem. Lett. 2010, 20, 5191-5194; Thomas, T. P.; et al., Biomacromolecules 2008, 9, 603-609; each herein incorporated by reference in its entirety)) (see, e.g., U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties). Its structure is characterized by a globular shape with a large number of peripheral branches amenable for chemical modifications and drug conjugation (see, e.g., Kukowska-Latallo, J. F.; et al., Cancer Res. 2005, 65, 5317-5324; Tomalia, D. A.; et al., Angew. Chem., Int. Ed. Engl. 1990, 29, 138-175; Svenson, S.; et al., Adv. Drug Delivery Rev. 2005, 57, 2106-2129; Baker, J. R., Jr.; Am. Soc. Hematol. Educ. Program 2009, 708-719; each herein incorporated by reference in its entirety)). In addition, its terminal branches are organized in a predefined orientation, and constitute a platform preferred for multivalent presentation of vancomycin (see, FIG. 2). This class of dendrimer conjugates is water soluble and structurally tunable for controlling the valency, allowing for the systematic investigation of valency-avidity correlation by surface plasmon resonance (SPR) spectroscopy.

Embodiments of the present invention allow rapid enrichment and detection of bacterial cells, and dramatically shorten the process. Furthermore, because of its ability to enrich bacterial cells into a small magnetic mass, in some embodiments, the isolation methods of the present invention can be coupled with simple detection methods such as fluorometry and fluorescent microscopy by which rapid and sensitive analysis can be performed in transfusion clinics.

Accordingly, the present invention provides compositions comprising dendrimer nanoparticles conjugated with Vancomycin molecules and/or Polymyxin (e.g., Polymyxin B, Polymyxin E) molecules. In certain embodiments, the dendrimer nanoparticles are conjugated with two or more Vancomycin molecules and/or Polymyxin B or E molecules (e.g., conjugated with an average of 2.3, 3.5 or 5.8 Vancomycin and/or Polymyxin molecules)). In some embodiments, iron oxide nanoparticles are coated with such dendrimer nanoparticles.

Such dendrimer nanoparticles are not limited to particular uses. In some embodiments, such compositions are used to treat disorders related to Gram-positive and/or Gram-negative bacteria. In some embodiments, such compositions are used to sequester and/or identify Gram-positive and/or Gram-negative bacteria. In some embodiments, such compositions are used to screen liquid samples (e.g., water samples, food samples, pharmaceutical samples, blood samples, blood platelet samples, etc.) for the presence of Gram-positive and/or Gram-negative bacteria. In some embodiments, such compositions are used to identify, sequester, and remove Gram-positive and/or Gram-negative bacteria from a liquid sample (e.g., water sample, food sample, pharmaceutical sample, blood sample, blood platelet sample, etc.).

Accordingly, the present invention provides dendrimer nanoparticles conjugated with Vancomycin and/or Polymyxin (e.g., Polymyxin B, Polymyxin E). In certain embodiments, such dendrimer nanoparticles are used to sequester and/or identify Gram-positive and/or Gram-negative bacteria, screen liquid samples (e.g., water samples, food samples, pharmaceutical samples, blood samples, blood platelet samples, etc.) for the presence of Gram-positive and/or Gram-negative bacteria, treat disorders associated with Gram-positive and/or Gram-negative bacteria, and/or identify, sequester, and remove Gram-positive and/or Gram-negative bacteria from a liquid sample (e.g., water sample, food sample, pharmaceutical sample, blood sample, blood platelet sample, etc.). In certain embodiments, iron oxide nanoparticles are coated with dendrimer nanoparticles conjugated with Vancomycin and/or Polymyxin. Experiments conducted during the course of developing embodiments for the present invention determined that compositions comprising dendrimer nanoparticles conjugated with Vancomycin are able to identify, target, sequester, and ameliorate the effect of Gram-positive and/or Gram-negative bacteria. In addition, such experiments further demonstrated that such compositions are able to identify, target, sequester, and ameliorate the effect of drug-resistant Gram-positive and/or Gram-negative bacteria. As such, the present invention is not limited to a particular use for such compositions.

In some embodiments, compositions comprising dendrimer nanoparticles conjugated with Vancomycin are used to treat disorders related to Gram-positive bacteria and/or drug resistant forms of Gram-positive bacteria. In some embodiments, compositions comprising dendrimer nanoparticles conjugated with Polymyxin B or E are used to treat disorders related to Gram-negative bacteria and/or drug resistant forms of Gram-negative bacteria. In some embodiments, iron oxide nanoparticles are coated with such dendrimer nanoparticles conjugated with Vancomycin alone, Polymyxin B or E alone, or in combination. The methods are not limited to treating particular disorders related to Gram-positive and/or Gram-negative bacteria and/or drug resistant Gram-positive and/or Gram-negative bacteria. Examples include, but are not limited to, pneumonia, endocarditis, bacteremia, sepsis and other forms of toxemia caused by Gram-positive and/or Gram-negative bacteria. In some embodiments, a subject having or suspected of having a disorder related to Gram-positive and/or Gram-negative bacteria is administered a composition comprising dendrimer nanoparticles conjugated with Vancomycin or Polymyxin B alone, or in combination. In such embodiments, upon such administration, the dendrimer nanoparticles locate and bind with the Gram-positive and/or Gram-negative bacteria via the conjugated Vancomycin and/or Polymyxin B, thereby ameliorating the effects of the Gram-positive and/or Gram-negative bacteria, and thereby treating the disorder. In some embodiments, additional agents are administered and/or co-administered with such compositions.

In some embodiments, compositions comprising dendrimer nanoparticles conjugated with Vancomycin or Polymyxin (e.g., Polymyxin B, Polymyxin E) alone, or in combination, are used to treat disorders related to Gram-positive and Gram-negative bacteria and/or drug resistant forms of Gram-positive and Gram-negative bacteria. In some embodiments, iron oxide nanoparticles are coated with such dendrimer nanoparticles conjugated with Vancomycin or Polymyxin (Polymyxin B, Polymyxin E) alone, or in combination. The methods are not limited to treating particular disorders related to Gram-positive and Gram-negative bacteria and/or drug resistant Gram-positive and Gram-negative bacteria. Examples include, but are not limited to, pneumonia, endocarditis, bacteremia, sepsis and other forms of toxemia caused by Gram-positive and Gram-negative bacteria. In some embodiments, a subject having or suspected of having a disorder related to Gram-positive and Gram-negative bacteria is administered a composition comprising dendrimer nanoparticles conjugated with Vancomycin or Polymyxin (e.g., Polymyxin B, Polymyxin E) alone, or in combination. In such embodiments, upon such administration, the dendrimer nanoparticles locate and bind with the Gram-positive and Gram-negative bacteria via the conjugated Vancomycin and/or Polymyxin (e.g., Polymyxin B, Polymyxin E), respectively, thereby ameliorating the effects of the Gram-positive and/or Gram-negative bacteria, and thereby treating the disorder. In some embodiments, additional agents are administered and/or co-administered with such compositions. Such agents include, but are not limited, to antibiotics (e.g., Gentamicin), and silver antibacterial agents.

In some embodiments, compositions comprising dendrimer nanoparticles conjugated with Vancomycin and imaging agents are used to identify Gram-positive bacteria within a sample. In some embodiments, compositions comprising dendrimer nanoparticles conjugated with two or more Vancomycin molecules (e.g., conjugated with an average of 2.3, 3.5 or 5.8 Vancomycin molecules) and imaging agents are used to identify Gram-positive bacteria within a sample. In some embodiments, compositions comprising dendrimer nanoparticles conjugated with Polymyxin (e.g., Polymyxin B, Polymyxin E) and imaging agents are used to identify Gram-negative bacteria within a sample. In some embodiments, compositions comprising dendrimer nanoparticles conjugated with two or more Polymyxin (e.g., Polymyxin B, Polymyxin E) molecules (e.g., conjugated with an average of 2.3, 3.5 or 5.8 Polymyxyin molecules) and imaging agents are used to identify Gram-negative bacteria within a sample. In some embodiments, compositions comprising dendrimer nanoparticles conjugated with Vancomycin, Polymyxin (e.g., Polymyxin B, Polymyxin E) and imaging agents are used to identify Gram-negative bacteria and Gram-negative bacteria within a sample. In some embodiments, compositions comprising dendrimer nanoparticles conjugated with two or more Vancomycin molecules (e.g., conjugated with an average of 2.3, 3.5 or 5.8 Vancomycin molecules), two or more Polymyxin (e.g., Polymyxin B, Polymyxin E) molecules (e.g., conjugated with an average of 2.3, 3.5 or 5.8 Polymyxyin molecules) and imaging agents are used to identify Gram-negative bacteria and Gram-negative bacteria within a sample. In some embodiments, iron oxide nanoparticles are coated with such dendrimer nanoparticles (e.g., conjugated with Vancomycin and/or Polymyxin) and imaging agents. In some embodiments, the Gram-positive and Gram-negative bacteria are drug-resistant Gram-positive and drug resistant Gram-negative bacteria. In some embodiments, the sample is a liquid sample. In some embodiments, the liquid sample is, for example, a water sample, a food sample, a pharmaceutical sample, a blood sample (e.g., contaminated blood), or blood product samples (e.g., a blood platelet sample). Examples of imaging agents include, but are not limited to, molecular dyes, fluorescein isothiocyanate (FITC), 6-TAMRA, acridine orange, and cis-parinaric acid. In some embodiments, the imaging agents are moleculear dyes from the alexa fluor (Molecular Probes) family of molecular dyes, Alexa Fluor 350 (blue), Alexa Fluor 405 (violet), Alexa Fluor 430 (green), Alexa Fluor 488 (cyan-green), Alexa Fluor 500 (green), Alexa Fluor 514 (green), Alexa Fluor 532 (green), Alexa Fluor 546 (yellow), Alexa Fluor 555 (yellow-green), Alexa Fluor 568 (orange), Alexa Fluor 594 (orange-red), Alexa Fluor 610 (red), Alexa Fluor 633 (red), Alexa Fluor 647 (red), Alexa Fluor 660 (red), Alexa Fluor 680 (red), Alexa Fluor 700 (red), and Alexa Fluor 750 (red). Additional examples of imaging agents include, but are not limited to, MRI contrast agents, gadolinium-diethylenetriaminepentacetate (Gd-DTPA), and gadolium-1,4,7,10-tetraazacyclododecane-1,4,7,10-tetraacetic acid (Gd-DOTA). In some embodiments, the method comprises administering to the liquid sample a composition comprising such dendrimers, and wherein upon binding with such Gram-positive and/or Gram-negative bacteria, the imaging agents are detected. In some embodiments, the methods are used to detect the presence of Gram-positive and/or Gram-negative bacteria in a food product (e.g., to ensure the food sample is free of Gram-positive and/or Gram-negative bacteria). In some embodiments, the methods are used to detect the presence of Gram-positive and/or Gram-negative bacteria in a blood sample (e.g., a blood sample to be used in a blood transfusion) (e.g., to ensure the blood sample is free of Gram-positive and/or Gram-negative bacteria). In some embodiments, the methods are used to detect the presence of Gram-positive and/or Gram-negative bacteria in a blood product sample (e.g., blood platelets) (e.g., to ensure the blood product sample is free of Gram-positive and/or Gram-negative bacteria). In some embodiments, the methods are used to detect the presence of Gram-positive and/or Gram-negative bacteria in a water sample (e.g., to ensure the water sample is free of Gram-positive and/or Gram-negative bacteria). In some embodiments, the methods are used to detect the presence of Gram-positive and/or Gram-negative bacteria in a pharmaceutical sample (e.g., to ensure the pharmaceutical sample is free of Gram-positive and/or Gram-negative bacteria). In some embodiments, the concentration of Gram-positive and/or Gram-negative bacteria is detectable based upon the amount of imaging agent detected. In some embodiments, the methods are used to characterize the presence or absence of a Gram-positive and/or Gram-negative bacterial disorder.

In some embodiments, compositions comprising iron oxide nanoparticles coated with dendrimer nanoparticles conjugated with Vancomycin are used to sequester Gram-positive bacteria from a sample. In some embodiments, compositions comprising iron oxide nanoparticles coated with dendrimer nanoparticles conjugated with Polymyxin (e.g., Polymyxin B, Polymyxin E) are used to sequester Gram-negative bacteria from a sample. In some embodiments, the Gram-positive and Gram-negative bacteria are drug-resistant Gram-positive and Gram-negative bacteria. In some embodiments, the sample is a liquid sample. In some embodiments, the liquid sample is, for example, a water sample, a food sample, a pharmaceutical sample, a blood sample (e.g., contaminated blood), or blood product samples (e.g., a blood platelet sample). In some embodiments, the methods comprise administering to the liquid sample a composition comprising such iron oxide nanoparticles coated with dendrimer, and wherein upon binding with such Gram-positive and/or Gram-negative bacteria, a magnetic field and/or centrifugation is applied to the sample resulting in a sequestering of the Gram-positive and/or Gram-negative bacteria. In some embodiments, the methods are used to screen for the presence of Gram-positive and/or Gram-negative bacteria in a food product (e.g., to screen the food sample for Gram-positive and/or Gram-negative bacterial contamination). In some embodiments, the methods are used to screen for the presence of Gram-positive and/or Gram-negative bacteria in a blood sample (e.g., a blood sample to be used in a blood transfusion) (e.g., to screen the blood sample for Gram-positive and/or Gram-negative bacterial contamination). In some embodiments, the methods are used to screen for the presence of Gram-positive and/or Gram-negative bacteria in a blood product sample (e.g., blood platelets) (e.g., to screen the blood product sample for Gram-positive and/or Gram-negative bacterial contamination). In some embodiments, the methods are used to screen for the presence of Gram-positive and/or Gram-negative bacteria in a water sample (e.g., to screen the water sample for Gram-positive and/or Gram-negative bacterial contamination). In some embodiments, the methods are used to screen for the presence of Gram-positive and/or Gram-negative bacteria in a pharmaceutical sample (e.g., to screen the pharmaceutical sample for Gram-positive and/or Gram-negative bacterial contamination). In some embodiments, compositions comprising iron oxide nanoparticles coated with dendrimer nanoparticles conjugated with Vancomycin are used to sequester Gram-positive bacteria from a sample. In some embodiments, compositions comprising iron oxide nanoparticles coated with dendrimer nanoparticles conjugated with Polymyxin (e.g., Polymyxin B, Polymyxin E) are used to sequester Gram-negative bacteria from a sample. In some embodiments, the Gram-positive and Gram-negative bacteria are drug-resistant Gram-positive and Gram-negative bacteria. In some embodiments, the sample is a liquid sample. In some embodiments, the liquid sample is, for example, a water sample, a food sample, a pharmaceutical sample, a blood sample (e.g., contaminated blood), or blood product samples (e.g., a blood platelet sample). In some embodiments, the methods comprise administering to the liquid sample a composition comprising such iron oxide nanoparticles coated with such a dendrimer (e.g., conjugated with Vancomycin and/or Polymyxin), and wherein upon binding with such Gram-positive and/or Gram-negative bacteria, a magnetic field and/or centrifugation is applied to the sample resulting in a sequestering of the Gram-positive and/or Gram-negative bacteria, and wherein the sequestered Gram-positive and/or Gram-negative bacteria are subsequently removed from the sample. In some embodiments, the methods are used to detect the presence of Gram-positive and/or Gram-negative bacteria in a food product (e.g., so as to alleviate a Gram-positive and/or Gram-negative bacteria contamination of the food sample). In some embodiments, the methods are used to sequester and remove Gram-positive and/or Gram-negative bacteria in a blood sample (e.g., a blood sample to be used in a blood transfusion) (e.g., so as to alleviate a Gram-positive and/or Gram-negative bacteria contamination of the blood sample). In some embodiments, the methods are used to sequester and remove the presence of Gram-positive and/or Gram-negative bacteria in a blood product sample (e.g., blood platelets) (e.g., so as to alleviate a Gram-positive and/or Gram-negative bacteria contamination of the blood product sample). In some embodiments, the methods are used to sequester and remove the presence of Gram-positive and/or Gram-negative bacteria in a water sample (e.g., so as to alleviate a Gram-positive and/or Gram-negative bacteria contamination of the water sample). In some embodiments, the methods are used to sequester and remove the presence of Gram-positive and/or Gram-negative bacteria in a pharmaceutical sample (e.g., so as to alleviate a Gram-positive and/or Gram-negative bacteria contamination of the pharmaceutical sample).

The compositions and methods of the present invention are not limited to the use of a particular type of dendrimer nanoparticle. Dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core are known (U.S. patent application Ser. No. 12/403,179; herein incorporated by reference in its entirety). In preferred embodiments, the protected core diamine is NH₂—CH₂—CH₂—NHPG. Molecular weight and the number of terminal groups increase exponentially as a function of generation (the number of layers) of the polymer. Different types of dendrimers can be synthesized based on the core structure that initiates the polymerization process.

The dendrimer core structures dictate several characteristics of the molecule such as the overall shape, density and surface functionality (see, e.g., Tomalia et al., Chem. Int. Ed. Engl., 29:5305 (1990)). Spherical dendrimers can have ammonia as a trivalent initiator core or ethylenediamine (EDA) as a tetravalent initiator core. Recently described rod-shaped dendrimers (see, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, ¹H nuclear magnetic resonance spectroscopy, size exclusion chromatography with multi-angle laser light scattering, ultraviolet spectrophotometry, capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describes methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

PAMAM dendrimers are highly branched, narrowly dispersed synthetic macromolecules with well-defined chemical structures. PAMAM dendrimers can be easily modified and conjugated with multiple functionalities such as targeting molecules, imaging agents, and drugs (Thomas et al. (2007) Poly(amidoamine) Dendrimer-based Multifunctional Nanoparticles, in Nanobiotechnology: Concepts, Methods and Perspectives, Merkin, Ed., Wiley-VCH; herein incorporated by reference in its entirety). They are water soluble, biocompatible, and cleared from the blood through the kidneys (Peer et al. (2007) Nat. Nanotechnol. 2:751-760; herein incorporated by reference in its entirety) which eliminates the need for biodegradability. Because of these desirable properties, PAMAM dendrimers have been widely investigated for drug delivery (Esfand et al. (2001) Drug Discov. Today 6:427-436; Patri et al. (2002) Curr. Opin. Chem. Biol. 6:466-471; Kukowska-Latallo et al. (2005) Cancer Res. 65:5317-5324; Quintana et al. (2002) Pharmaceutical Res. 19:1310-1316; Thomas et al. (2005) J. Med. Chem. 48:3729-3735; each herein incorporated by reference in its entirety), gene therapy (KukowskaLatallo et al. (1996) PNAS 93:4897-4902; Eichman et al. (2000) Pharm. Sci. Technolo. Today 3:232-245; Luo et al. (2002) Macromol. 35:3456-3462; each herein incorporated by reference in its entirety), and imaging applications (Kobayashi et al. (2003) Bioconj. Chem. 14:388-394; herein incorporated by reference in its entirety).

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polyeation non-covalently coupled to the polynucleotide.

Dendrimer-antibody conjugates for use in in vitro diagnostic applications have previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control Rel. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Hom, Science 261:534 (1993)). This allows the controlled degradation of the polymer to release therapeutics at the disease site and provides a mechanism for an external trigger to release the therapeutic agents.

In experiments conducted during the course of developing embodiments for the present invention, dendrimer nanoparticles (e.g., PAMAM dendrimer nanoparticles) conjugated with an agent were developed for purposes of, for example, identifying and/or sequestering Gram-positive bacteria. The present invention is not limited to a particular type of agent for identifying and/or sequestering Gram-positive bacteria. In some embodiments, the agent for identifying and/or sequestering Gram-positive bacteria is Vancomycin.

In some embodiments, metal nanoparticles are coated or encapsulated with dendrimers conjugated with agents for identifying and/or sequestering Gram-positive bacteria (e.g., Vancomycin) and/or Gram-negative bacteria (e.g., Polymyxin (e.g., Polymyxin B, Polymyxin E)). The present invention is not limited to particular types of metal nanoparticles.

In some embodiments, the metal nanoparticle is iron oxide (Fe(II); Fe(III)). The present invention is not limited to a particular manner of coating or encapsulating the metal nanoparticles with such dendrimers. In some embodiments, the dendrimers are directly conjugated to the metal nanoparticles (e.g., iron oxide). In some embodiments, a layer-by-layer (LbL) self-assembly method is utilized in combination with dendrimer synthesis chemistry in order to generate dendrimers (conjugated with Vancomycin) comprising iron oxide nanoparticles (NPs) of the present invention (see, e.g., U.S. patent application Ser. No. 11/827,637; herein incorporated by reference in its entirety). In some embodiments, the composition is formed via charged interactions between the iron oxide nanoparticles and the dendrimer. In some embodiments, the composition is formed by incubating the dendrimer and iron oxide nanoparticles in a methanol solution containing acetic anhydride. In some embodiments, the metal nanoparticles (e.g., iron oxide nanoparticles) are conjugated to the dendrimer. In some embodiments, the conjugation comprises covalent bonds, ionic bonds, metallic bonds, hydrogen bonds, Van der Waals bonds, ester bonds or amide bonds.

The present invention is not limited to a particular manner of conjugating an agent for identifying and/or sequestering Gram-positive bacteria (e.g., Vancomycin) and/or Gram-negative bacteria (e.g., Polymyxin (e.g., Polymyxin B, Polymyxin E)) with a dendrimer nanoparticle. In some embodiments, an agent for identifying and/or sequestering Gram-positive bacteria (e.g., Vancomycin) and/or Gram-negative bacteria (e.g., Polymyxin (e.g., Polymyxin B, Polymyxin E)) is directly conjugated with a dendrimer (e.g., via terminal amine groups). In some embodiments, an agent for identifying and/or sequestering Gram-positive bacteria (e.g., Vancomycin) and/or Gram-negative bacteria (e.g., Polymyxin (e.g., Polymyxin B, Polymyxin E)) is conjugated with a dendrimer via linkage agents. Examples of such linkage agents include, but are not limited to, thiol groups, diene groups, dieneophile groups, and alkene groups.

The present invention is not limited to a particular manner of conjugating a bacteria-targeting agent to the dendrimer nanoparticle (e.g., at the C-terminus of Vancomycin). In some embodiments, Vancomycin is directly conjugated with a dendrimer at its C-terminus, resorcinol or vancosamine residue. For example, such conjugation methods are frequently achieved without loss of cell wall binding affinity (see, e.g., Long, D.; et al., J. Antibiot. 2008, 61, 603-14; Rao, J.; et al., Chem. Biol. (Cambridge, Mass., U.S.) 1999, 6, 353-59; Griffin, J. H.; et al., J. Am. Chem. Soc. 2003, 125, 6517-31; Arimoto, H.; et al., Tetrahedron Lett. 2001, 42, 3347-50; Leadbetter, M.; et al., J Antibiot (Tokyo) 2004, 57, 326-36; each herein incorporated by reference in its entirety). In some embodiments, Vancomycin is conjugated with a dendrimer via other tethering groups. Examples of such linkage groups include, but are not limited to, amide, carbamate, ester, amine, and di-sulfide groups.

In some embodiments, an agent for identifying and/or sequestering Gram-positive bacteria (e.g., Vancomycin) and/or Gram-negative bacteria (e.g., Polymyxin (e.g., Polymyxin B, Polymyxin E)) is conjugated with a dendrimer is facilitated by use of triazine molecules that are linked to functional components and used for one-step (e.g., click chemistry) addition to terminal arms of dendrimers. Click chemistry involves, for example, the coupling of two different moieties (e.g., a dendrimer conjugation ligand and an agent for identifying and/or sequestering Gram-positive bacteria (e.g., Vancomycin)) via a 1,3-dipolar cycloaddition reaction between an alkyne moiety (or equivalent thereof) on the surface of the first moiety and an azide moiety (or equivalent thereof) (or any active end group such as, for example, a primary amine end group, a hydroxyl end group, a carboxylic acid end group, a thiol end group, etc.) on the second moiety. Click chemistry is an attractive coupling method because, for example, it can be performed with a wide variety of solvent conditions including aqueous environments. For example, the stable triazole ring that results from coupling the alkyne with the azide is frequently achieved at quantitative yields and is considered to be biologically inert (see, e.g., Rostovtsev, V. V.; et al., Angewandte Chemie-International Edition 2002, 41, (14), 2596; Wu, P.; et al., Angewandte Chemie-International Edition 2004, 43, (30), 3928-3932; each herein incorporated by reference in their entireties). As examples of antibody conjugation ligands include, but are not limited to, alkyne groups (e.g., cyclooctyne, fluorinated cyclooctyne, alkyne), in some embodiments, the dendrimer conjugation ligand is an azide group (e.g., for purposes of facilitating a 1,3-dipolar cycloaddition reaction between the dendrimer conjugation ligand and the agent for identifying and/or sequestering Gram-positive bacteria (e.g., Vancomycin)).

In some embodiments, conjugation between a dendrimer (e.g., a terminal arm of a dendrimer) and an agent for identifying and/or sequestering Gram-positive bacteria (e.g., Vancomycin) is accomplished during a “one-pot” reaction. The term “one-pot synthesis reaction” or equivalents thereof, e.g., “1-pot”, “one pot”, etc., refers to a chemical synthesis method in which all reactants are present in a single vessel. Reactants may be added simultaneously or sequentially, with no limitation as to the duration of time elapsing between introduction of sequentially added reactants. In some embodiments, a one-pot reaction occurs wherein a hydroxyl-terminated dendrimer (e.g., HO-PAMAM dendrimer) is reacted with one or more functional ligands (e.g., an agent for identifying and/or sequestering Gram-positive bacteria (e.g., Vancomycin)) (e.g., a therapeutic agent, a pro-drug, a trigger agent, a targeting agent, an imaging agent) in one vessel, such conjugation being facilitated by ester coupling agents (e.g., 2-chloro-1-methylpyridinium iodide and 4-(dimethylamino) pyridine) (see, e.g., U.S. Provisional Patent App. No. 61/226,993, herein incorporated by reference in its entirety).

In some embodiments, the agent for identifying and/or sequestering Gram-positive bacteria (e.g., Vancomycin) is conjugated with the dendrimer and/or triazine compound via a trigger agent. The present invention is not limited to particular types or kinds of trigger agents.

In some embodiments, sustained release (e.g., slow release over a period of 24-48 hours) of the ligand is accomplished through conjugating the agent for identifying and/or sequestering Gram-positive bacteria (e.g., Vancomycin) and/or Gram-negative bacteria (e.g., Polymyxin (e.g., Polymyxin B, Polymyxin E)) (e.g., directly) (e.g., indirectly through one or more additional functional groups) to a trigger agent that slowly degrades in a biological system (e.g., amide linkage, ester linkage, ether linkage). In some embodiments, constitutively active release of the agent is accomplished through conjugating the agent to a trigger agent that renders the agent constitutively active in a biological system (e.g., amide linkage, ether linkage).

In some embodiments, release of an agent for identifying and/or Gram-positive bacteria (e.g., Vancomycin) and/or Gram-negative bacteria (e.g., Polymyxin (e.g., Polymyxin B, Polymyxin E)) under specific conditions is accomplished through conjugating the agent (e.g., directly) (e.g., indirectly through one or more additional functional groups) to a trigger agent that degrades under such specific conditions (e.g., through activation of a trigger molecule under specific conditions that leads to release of the agent). For example, once a conjugate (e.g., an agent for identifying and/or sequestering Gram-positive bacteria (e.g., Vancomycin) and/or Gram-negative bacteria (e.g., Polymyxin (e.g., Polymyxin B, Polymyxin E)) conjugated with a trigger agent) arrives at a target site in a subject (e.g., Gram-positive bacteria and/or Gram-negative bacteria), components in the target site interact with the trigger agent thereby initiating cleavage of the agent (e.g., Vancomycin and/or Polymyxin) from the trigger agent. In some embodiments, the trigger agent is configured to degrade (e.g., release the agent) upon exposure to a particular factor (e.g., hypoxia and pH, an enzyme (e.g., glucuronidase and/or plasmin), a cathepsin, a matrix metalloproteinase, a hormone receptor (e.g., integrin receptor, hyaluronic acid receptor, luteinizing hormone-releasing hormone receptor, etc.), cancer and/or tumor specific DNA sequence), an inflammatory associated factor (e.g., chemokine, cytokine, etc.) or other moiety.

In some embodiments, the present invention provides an agent for identifying and/or sequestering Gram-positive bacteria (e.g., Vancomycin) and/or Gram-negative bacteria (e.g., Polymyxin (e.g., Polymyxin B, Polymyxin E))conjugated with a trigger agent that is sensitive to (e.g., is cleaved by) hypoxia (e.g., indolequinone). Hypoxia is a feature of several disease states, including cancer, inflammation and rheumatoid arthritis, as well as an indicator of respiratory depression (e.g., resulting from analgesic drugs).

The concept of pro-drug systems in which the pharmacophores of drugs are masked by reductively cleavable groups has been widely explored by many research groups and pharmaceutical companies (see, e.g., Beall, H. D., et al., Journal of Medicinal Chemistry, 1998. 41(24): p. 4755-4766; Ferrer, S., D. P. Naughton, and M. D. Threadgill, Tetrahedron, 2003. 59(19): p. 3445-3454; Naylor, M. A., et al., Journal of Medicinal Chemistry, 1997. 40(15): p. 2335-2346; Phillips, R. M., et al., Journal of Medicinal Chemistry, 1999. 42(20): p. 4071-4080; Zhang, Z., et al., Organic & Biomolecular Chemistry, 2005. 3(10): p. 1905-1910; each of which are herein incorporated by reference in their entireties). Several such hypoxia activated pro-drugs have been advanced to clinical investigations, and work in relevant oxygen concentrations to prevent cerebral damage. The present invention is not limited to particular hypoxia activated trigger agents. In some embodiments, the hypoxia activated trigger agents include, but are not limited to, indolequinones, nitroimidazoles, and nitroheterocycles (see, e.g., Damen, E. W. P., et al., Bioorganic & Medicinal Chemistry, 2002. 10(1): p. 71-77; Hay, M. P., et al., Journal of Medicinal Chemistry, 2003. 46(25): p. 5533-5545; Hay, M. P., et al., Journal of the Chemical Society-Perkin Transactions 1, 1999(19): p. 2759-2770; each herein incorporated by reference in their entireties).

In some embodiments, the trigger agent is sensitive to (e.g., is cleaved by) and/or associates with a tumor-associated enzyme. For example, in some embodiments, the trigger agent that is sensitive to (e.g., is cleaved by) and/or associates with a glucuronidase. Glucuronic acid can be attached to several anticancer drugs via various linkers. These anticancer drugs include, but are not limited to, doxorubicin, paclitaxel, docetaxel, 5-fluorouracil, 9-aminocamtothecin, as well as other drugs under development. These pro-drugs are generally stable at physiological pH and are significantly less toxic than the parent drugs.

In some embodiments, the trigger agent is sensitive to (e.g., is cleaved by) and/or associates with brain enzymes. For example, trigger agents such as indolequinone are reduced by brain enzymes such as, for example, diaphorase (DT-diaphorase) (see, e.g., Damen, E. W. P., et al., Bioorganic & Medicinal Chemistry, 2002. 10(1): p. 71-77; herein incorporated by reference in its entirety).

In some embodiments, the trigger agent is sensitive to (e.g., is cleaved by) and/or associates with a protease. The present invention is not limited to any particular protease. In some embodiments, the protease is a cathepsin. In some embodiments, a trigger comprises a Lys-Phe-PABC moiety (e.g., that acts as a trigger). In some embodiments, utilization of a 1,6-elimination spacer/linker is utilized (e.g., to permit release of the agent (e.g., Vancomycin and/or Polymyxin) post activation of trigger).

In some embodiments, the trigger agent is sensitive to (e.g., is cleaved by) and/or associates with plasmin. The serine protease plasmin is over expressed in many human tumor tissues. Tripeptide specifiers (e.g., including, but not limited to, Val-Leu-Lys) have been identified and linked to anticancer drugs through elimination or cyclization linkers.

In some embodiments, the trigger agent is sensitive to (e.g., is cleaved by) and/or associates with a matrix metalloproteases (MMPs). In some embodiments, the trigger agent is sensitive to (e.g., is cleaved by) and/or that associates with β-Lactamase (e.g., a β-Lactamase activated cephalosporin-based pro-drug).

In some embodiments, the trigger agent is sensitive to (e.g., is cleaved by) and/or activated by a receptor (e.g., expressed on a target cell (e.g., Gram-positive bacteria and/or Gram-negative bacteria)). In some embodiments, dendrimers conjugated (e.g., directly or indirectly (e.g., via a triazine compound)) with an agent for identifying and/or sequestering Gram-positive bacteria (e.g., Vancomycin) and/or Gram-negative bacteria (e.g., Polymyxin (e.g., Polymyxin B, Polymyxin E)) are conjugated with additional targeting agents. The present invention is not limited to any particular additional targeting agents. In some embodiments, such targeting agents are conjugated to a dendrimer (e.g., directly or indirectly) for delivery to desired body regions (e.g., to the central nervous system (CNS); to a tumor). The targeting agents are not limited to targeting specific body regions.

In some embodiments, the additional targeting agent is a moiety that has affinity for a tumor associated factor. For example, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)).

The present invention is not limited to cancer and/or tumor targeting agents. Indeed, multifunctional dendrimers can be targeted (e.g., via a linker conjugated to the dendrimer wherein the linker comprises a targeting agent) to a variety of target cells or tissues (e.g., to a biologically relevant environment) via conjugation to an appropriate targeting agent. For example, in some embodiments, the targeting agent is a moiety that has affinity for an inflammatory factor (e.g., a cytokine or a cytokine receptor moiety (e.g., TNF-α receptor)). In some embodiments, the targeting agent is a sugar, peptide, antibody or antibody fragment, hormone, hormone receptor, or the like.

In some embodiments of the present invention, the additional targeting agent includes, but is not limited to an antibody, receptor ligand, hormone, vitamin, and antigen, however, the present invention is not limited by the nature of the targeting agent. In some embodiments, the antibody is specific for a disease-specific antigen. In some embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In some embodiments, the receptor ligand is folic acid.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some embodiments, the additional targeting agent is an antibody. In some embodiments, the antibodies recognize, for example, tumor-specific epitopes (e.g., TAG-72 (See, e.g., Kjeldsen et al., Cancer Res. 48:2214-2220 (1988); U.S. Pat. Nos. 5,892,020; 5,892,019; and 5,512,443; each herein incorporated by reference in their entireties); human carcinoma antigen (See, e.g., U.S. Pat. Nos. 5,693,763; 5,545,530; and 5,808,005; each herein incorporated by reference in their entireties); TP1 and TP3 antigens from osteocarcinoma cells (See, e.g., U.S. Pat. No. 5,855,866; herein incorporated by reference in its entirety); Thomsen-Friedenreich (TF) antigen from adenocarcinoma cells (See, e.g., U.S. Pat. No. 5,110,911; herein incorporated by reference in its entirety); “KC-4 antigen” from human prostrate adenocarcinoma (See, e.g., U.S. Pat. Nos. 4,708,930 and 4,743,543; each herein incorporated by reference in their entireties); a human colorectal cancer antigen (See, e.g.,

U.S. Pat. No. 4,921,789; herein incorporated by reference in its entirety); CA125 antigen from cystadenocarcinoma (See, e.g., U.S. Pat. No. 4,921,790; herein incorporated by reference in its entirety); DF3 antigen from human breast carcinoma (See, e.g., U.S. Pat. Nos. 4,963,484 and 5,053,489; each herein incorporated by reference in their entireties); a human breast tumor antigen (See, e.g., U.S. Pat. No. 4,939,240: herein incorporated by reference in its entirety); p97 antigen of human melanoma (See, e.g., U.S. Pat. No. 4,918,164: herein incorporated by reference in its entirety); carcinoma or orosomucoid-related antigen (CORA) (See, e.g., U.S. Pat. No. 4,914,021; herein incorporated by reference in its entirety); a human pulmonary carcinoma antigen that reacts with human squamous cell lung carcinoma but not with human small cell lung carcinoma (See, e.g., U.S. Pat. No. 4,892,935; herein incorporated by reference in its entirety); T and Tn haptens in glycoproteins of human breast carcinoma (See, e.g., Springer et al., Carbohydr. Res. 178:271-292 (1988); herein incorporated by reference in its entirety), MSA breast carcinoma glycoprotein termed (See, e.g., Tjandra et al., Br. J. Surg. 75:811-817 (1988); herein incorporated by reference in its entirety); MFGM breast carcinoma antigen (See, e.g., Ishida et al., Tumor Biol. 10:12-22 (1989); herein incorporated by reference in its entirety); DU-PAN-2 pancreatic carcinoma antigen (See, e.g., Lan et al., Cancer Res. 45:305-310 (1985); herein incorporated by reference in its entirety); CA125 ovarian carcinoma antigen (See, e.g., Hanisch et al., Carbohydr. Res. 178:29-47 (1988); herein incorporated by reference in its entirety); YH206 lung carcinoma antigen (See, e.g., Hinoda et al., (1988) Cancer J. 42:653-658 (1988); herein incorporated by reference in its entirety).

In some embodiments, the additional targeting agents target the central nervous system (CNS). In some embodiments, where the additional targeting agent is specific for the CNS, the targeting agent is transferrin (see, e.g., Daniels, T. R., et al., Clinical Immunology, 2006. 121(2): p. 159-176; Daniels, T. R., et al., Clinical Immunology, 2006. 121(2): p. 144-158; each herein incorporated by reference in their entireties). Transferrin has been utilized as a targeting vector to transport, for example, drugs, liposomes and proteins across the blood-brain barrier (BBB) by receptor mediated transcytosis (see, e.g., Smith, M. W. and M. Gumbleton, Journal of Drug Targeting, 2006. 14(4): p. 191-214; herein incorporated by reference in its entirety). In some embodiments, the targeting agents target neurons within the central nervous system (CNS). In some embodiments, where the targeting agent is specific for neurons within the CNS, the targeting agent is a synthetic tetanus toxin fragment (e.g., a 12 amino acid peptide (Tet 1) (HLNILSTLWKYR)) (see, e.g., Liu, J. K., et al., Neurobiology of Disease, 2005. 19(3): p. 407-418; herein incorporated by reference in its entirety).

In some embodiments, the dendrimer conjugated (e.g., directly or indirectly (e.g., via a triazine compound)) with an agent for identifying and/or sequestering Gram-positive bacteria (e.g., Vancomycin) and/or Gram-negative bacteria (e.g., Polymyxin (e.g., Polymyxin B, Polymyxin E)) is further conjugated to an imaging agent. A multiplicity of imaging agents find use in the present invention. In some embodiments, a multifunctional dendrimer comprises at least one imaging agent that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

In some embodiments, once a component(s) of a targeted multifunctional dendrimer has attached to (or been internalized into) a target cell, one or more modules serves to image its location. In some embodiments, chelated paramagnetic ions, such as Gd(III)-diethylenetriaminepentaacetic acid (Gd(III)-DTPA), are conjugated to the multifunctional dendrimer. Other paramagnetic ions that may be useful in this context include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof.

Dendrimeric gadolinium contrast agents have even been used to differentiate between benign and malignant breast tumors using dynamic MRI, based on how the vasculature for the latter type of tumor images more densely (see, e.g., Adam et al., Ivest. Rad. 31:26 (1996); herein incorporated by reference in its entirety). Thus, MRI provides a particularly useful imaging system of the present invention.

Multifunctional dendrimers allow functional microscopic imaging of tumors and provide improved methods for imaging. The methods find use in vivo, in vitro, and ex vivo. For example, in one embodiment, dendrimer functional groups are designed to emit light or other detectable signals upon exposure to light. Although the labeled functional groups may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (See, e.g., Farkas et al., SPEI 2678:200 (1997); herein incorporated by reference in its entirety). In embodiments where the imaging agents are present in deeper tissue, longer wavelengths in the Near-infrared (NMR) are used (See e.g., Lester et al., Cell Mol. Biol. 44:29 (1998); herein incorporated by reference in its entirety). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce when excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments, biosensor-comprising pro-drug complexes are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic biosensors for pH, oxygen concentration, Ca²+ concentration, and other physiologically relevant analytes.

In these embodiments, fluorescent groups such as fluorescein are employed in the imaging agent. Fluorescein is easily attached to the dendrimer surface via the isothiocyanate derivatives, available from MOLECULAR PROBES, Inc. This allows the multifunctional dendrimer or components thereof to be imaged with the cells via confocal microscopy. Sensing of the effectiveness of the multifunctional dendrimer or components thereof is preferably achieved by using fluorogenic peptide enzyme substrates. For example, apoptosis caused by an agent results in the production of the peptidase caspase-1 (ICE). CALBIOCHEM sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (See, e.g., Talanian et al., J. Biol. Chem., 272: 9677 (1997); herein incorporated by reference in its entirety). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm). In some embodiments, the lysine end of the peptide is linked to pro-drug complex, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (see, e.g., Abrams et al., Development 117:29 (1993); herein incorporated by reference in its entirety) and cis-parinaric acid, sensitive to the lipid peroxidation that accompanies apoptosis (see, e.g., Hockenbery et al., Cell 75:241 (1993); herein incorporated by reference in its entirety). It should be noted that the peptide and the fluorescent dyes are merely exemplary. It is contemplated that any peptide that effectively acts as a substrate for a caspase produced as a result of apoptosis finds use with the present invention.

Functionalized nanoparticles (e.g., dendrimers) often contain moieties (including but not limited to ligands, functional ligands, conjugates, therapeutic agents, targeting agents, imaging agents, fluorophores) that are conjugated to the periphery. Such moieties may for example be conjugated to one or more dendrimer branch termini. Classical multi-step conjugation strategies used during the synthesis of functionalized dendrimers generate a stochastic distribution of products with differing numbers of ligands attached per dendrimer molecule, thereby creating a population of dendrimers with a wide distribution in the numbers of ligands attached. The low structural uniformity of such dendrimer populations negatively affects properties such as therapeutic potency, pharmacokinetics, or effectiveness for multivalent targeting. Difficulties in quantifying and resolving such populations to yield samples with sufficient structural uniformity can pose challenges. However, in some embodiments, use of separation methods (e.g., reverse phase chromatography) customized for optimal separation of dendrimer populations in conjunction with peak fitting analysis methods allows isolation and identification of subpopulations of functionalized dendrimers with high structural uniformity (see, e.g., U.S. Provisional Pat. App. No. 61/237,172; herein incorporated by reference in its entirety). In certain embodiments, such methods and systems provide a dendrimer product made by the process comprising: a) conjugation of at least one ligand type to a dendrimer (e.g., an agent for identifying and/or sequestering Gram-positive bacteria (e.g., Vancomycin) and/or Gram-negative bacteria (Polymyxin (e.g., Polymyxin B, Polymyxin E)) to yield a population of ligand-conjugated dendrimers; b) separation of the population of ligand-conjugated dendrimers with reverse phase HPLC to result in subpopulations of ligand-conjugated dendrimers indicated by a chromatographic trace; and c) application of peak fitting analysis to the chromatographic trace to identify subpopulations of ligand-conjugated dendrimers wherein the structural uniformity of ligand conjugates per molecule of dendrimer within said subpopulation is, e.g., approximately 80% or more.

In some embodiments, dendrimers conjugated with an agent for identifying and/or sequestering Gram-positive bacteria (e.g., Vancomycin) and/or Gram-negative bacteria (Polymyxin (e.g., Polymyxin B, Polymyxin E)) are further conjugated with an additional therapeutic agent. The present invention is not limited by the type of therapeutic agent delivered via multifunctional dendrimers of the present invention. For example, a therapeutic agent may be any agent selected from the group comprising, but not limited to, a pain relief agent, a pain relief agent antagonist, a chemotherapeutic agent, an anti-oncogenic agent, an anti-angiogenic agent, a tumor suppressor agent, an anti-microbial agent, or an expression construct comprising a nucleic acid encoding a therapeutic protein. Examples of such therapeutic agents include, but are not limited to, the therapeutic agents recited in, for example, U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos. 61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties.

Where clinical applications are contemplated, in some embodiments of the present invention, the dendrimer conjugates are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In some embodiments, the dendrimer conjugates are used in conjunction with appropriate salts and buffers to render delivery of the compositions in a stable manner to allow for uptake by target cells. Buffers also are employed when the dendrimer conjugates are introduced into a patient. Aqueous compositions comprise an effective amount of the dendrimer conjugates to cells dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with vectors, cells, or tissues, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active dendrimer conjugates may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. In some embodiments, a therapeutic agent is released from dendrimer conjugates within a target cell (e.g., within an endosome). This type of intracellular release (e.g., endosomal disruption of a linker-therapeutic conjugate) is contemplated to provide additional specificity for the compositions and methods of the present invention. The present invention provides dendrimers with multiple (e.g., 100-150) reactive sites for the conjugation of linkers and/or functional groups comprising, but not limited to, therapeutic agents, targeting agents, imaging agents and biological monitoring agents.

The compositions and methods of the present invention are contemplated to be equally effective whether or not the dendrimer conjugates of the present invention comprise a fluorescein (e.g. FITC) imaging agent. Thus, each functional group present in a dendrimer composition is able to work independently of the other functional groups. Thus, the present invention provides dendrimer conjugates that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, dendrimer conjugates are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The dendrimer conjugates also may be formulated as inhalants for the treatment of lung cancer and such like.

### EXAMPLES

The following examples are provided in order to demonstrate and further illustrate certain preferred embodiments and aspects of the present invention and are not to be construed as limiting the scope thereof.

### Example 1

Previous experiments involving dendrimer related technologies are located in U.S. Pat. Nos. 6,471,968, 7,078,461; U.S. patent application Ser. Nos. 09/940,243, 10/431,682, 11,503,742, 11,661,465, 11/523,509, 12/403,179, 12/106,876, 11/827,637, 10/039,393, 10/254,126, 09/867,924, 12/570,977, and 12/645,081; U.S. Provisional Patent Application Ser. Nos.61/256,699, 61/226,993, 61/140,480, 61/091,608, 61/097,780, 61/101,461, 61/251,244, 60/604,321, 60/690,652, 60/707,991, 60/208,728, 60/718,448, 61/035,949, 60/830,237, and 60/925,181; and International Patent Application Nos. PCT/US2010/051835, PCT/US2010/050893; PCT/US2010/042556, PCT/US2001/015204, PCT/US2005/030278, PCT/US2009/069257, PCT/US2009/036992, PCT/US2009/059071, PCT/US2007/015976, and PCT/US2008/061023, each herein incorporated by reference in their entireties.

### Example 2

This example describes the design and synthesis of vancomycin-conjugated PAMAM dendrimers. The current dendrimer platform used for vancomycin conjugation is based on G5 PAMAM dendrimer (G5-NH₂) (see, e.g., Tomalia, D. A.; et al., Angew. Chem., Int. Ed. Engl. 1990, 29, 138-175; Liang, C.; et al., Prog. Polym. Sci. 2005, 30, 385-402; each herein incorporated by reference in its entirety)). This dendrimer generation provides a sufficient number of peripheral branches (theoretically 128) (see, e.g., Mullen, D.; et al., Chem. Eur. J. 2010, 16, 10675-10678; Mullen, D. G.; et al., ACS Nano 2010, 4, 657-670; each herein incorporated by reference in its entirety)), each terminated with a primary amine amenable to covalent conjugation with a variety of targeting ligands and drug molecules (see, e.g., Majoros, I.; Baker Jr, J. Dendrimer-Based Nanomedicine. Pan Stanford: Hackensack, N.J., 2008; p 436; Cloninger, M. J.; Curr. Opin. Chem. Biol. 2002, 6, 742-748; Esfand, R.; et al., Drug Discovery Today 2001, 6, 427-436′ Medina, S. H.; et al., Chem. Rev. (Washington, D.C., U.S.) 2009, 109, 3141-3157; each herein incorporated by reference in its entirety)). The approach in the design of vancomycin-presenting G5 dendrimer conjugates involves two molecular parameters pertinent to vancomycin (Scheme 1): i) the C-terminus as the position for vancomycin attachment and ii) variation of the multivalency. First, a series of Ac-G5-(V)ₙ conjugates I-V were prepared, each of which contained multiple vancomycin molecules at a variable mean valency. This series was prepared by preactivation of vancomycin by benzotriazol-1-yl-oxytripyrrolidinophosphonium hexafluorophosphate (PyBOP) and N-hydroxybenzotriazole (HOBt) (see, e.g., Griffin, J. H.; et al., J. Am. Chem. Soc. 2003, 125, 6517-6531; Long, D.; et al., J. Antibiot. 2008, 61, 603-614; each herein incorporated by reference in its entirety)), and subsequently by conjugation to the G5 PAMAM dendrimer through an amide bond. After this coupling step, each of the unreacted primary amines on the dendrimer was converted to an N-acetyl amide after treatment with acetic anhydride, making the dendrimer surface neutral. By varying the molar ratio of the two reactants ([Vancomycin]/[G5-NH₂]=2, 4, 6, 8, and 10), it was possible to control the vancomycin valency that led to five different mean valencies of Ac-G5-(V)ₙ (n=1.2, 2.3, 3.5, 5.8, and 8.3).

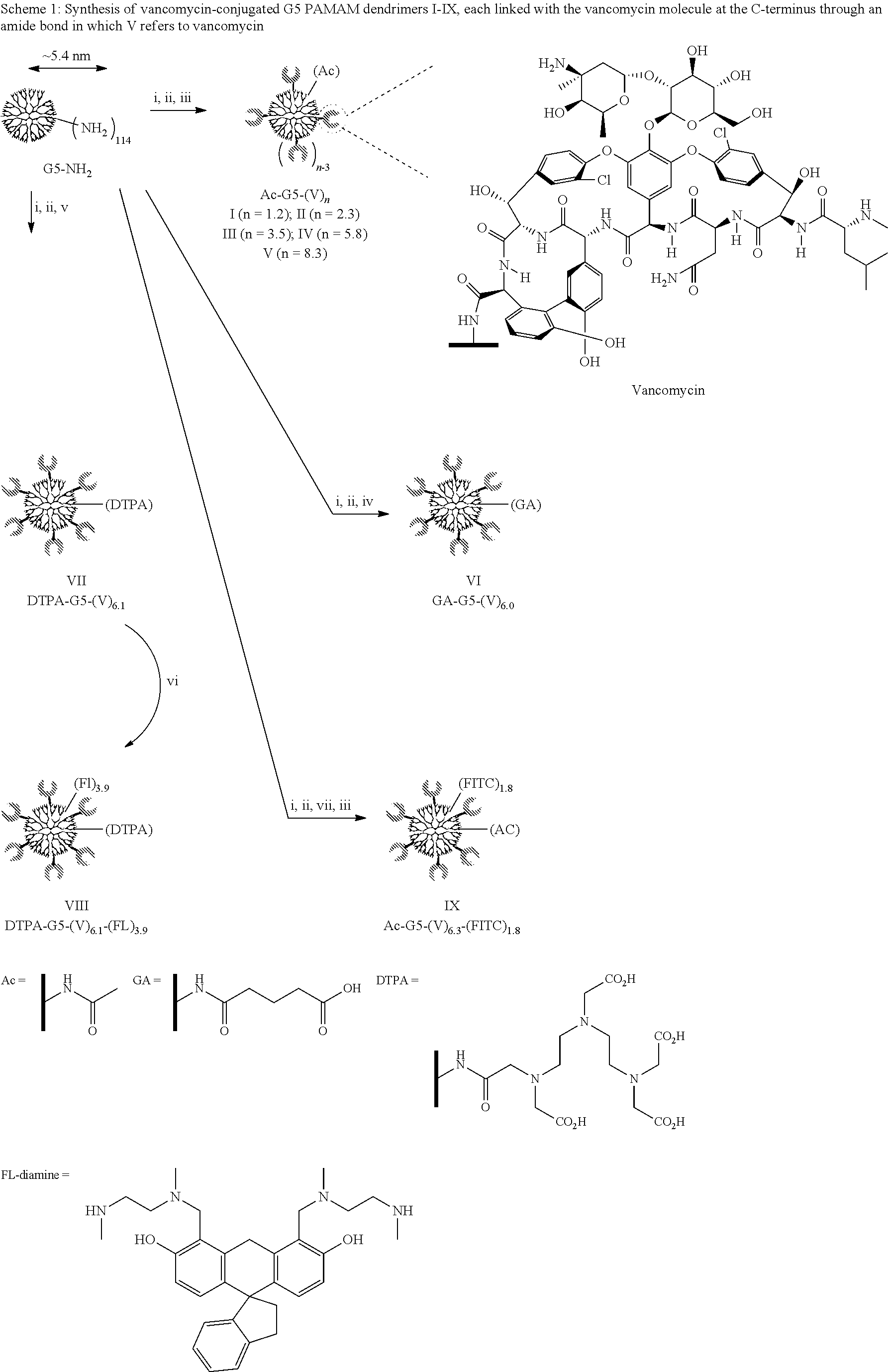

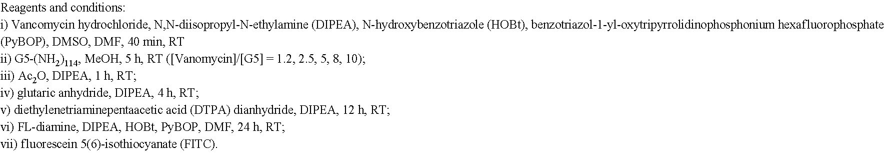

This synthetic method was slightly modified to alter the physicochemical property of the dendrimer surface and also to introduce other functional moieties on the surface for enabling multifunctional applications. Thus two negatively-charged conjugates, VI GA-G5-(V)6 and VII DTPA-G5-(V)_{6.1}, were prepared by replacing the acetic anhydride used in the second step with glutaric anhydride or diethylenetriaminepentaacetic acid (DTPA), respectively. The DTPA group present in the latter conjugate allows metal chelation with Gd (III) ions (see, e.g., Caravan, P.; et al., Chem. Rev. (Washington, D.C., U.S.) 1999, 99, 2293-2352; herein incorporated by reference in its entirety)) and provides magnetic resonance imaging (MRI) capability potentially applicable for in vivo detection and imaging of bacterial cells. Those carboxylic acids localized on the dendrimer surface of VI and VII could be used for further derivatization, as illustrated by the synthesis of conjugate VIII, DTPA-G5-(V)_{6.1}-(Fl)_{3.9}, which carries fluorescein imaging molecules (FL-diamine; excitation/emission wavelength=494 nm/518 nm) (see, e.g., Nolan, E. M.; et al., J. Am. Chem. Soc. 2006, 128, 15517-15528; herein incorporated by reference in its entirety)). In another approach for conjugation with fluorescent dye molecules, the primary amines of the dendrimer reacted directly with fluorescein isothiocyanate (FITC) prior to the last exhaustive N-acetylation step that led to the conjugate IX Ac-G5-(V)_{6.3}-(FITC)_{1.8}.

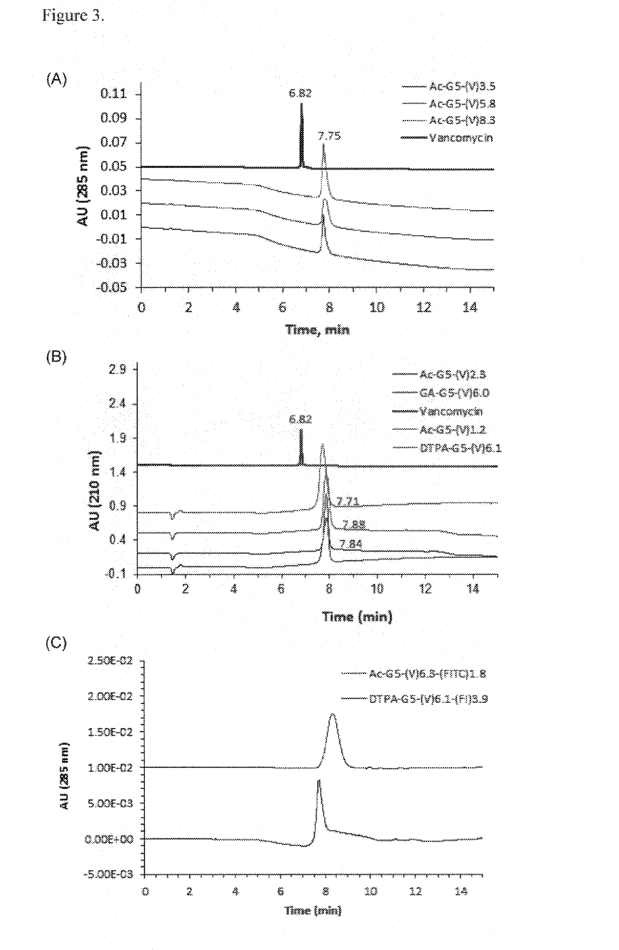

Purification of each vancomycin-conjugated dendrimer was performed by dialysis using a membrane tubing (molecular weight cut off or MWCO=10 kDa) until its purity was greater than 95% as determined by the HPLC method (see, FIG. 3). Each of these conjugates I-IX was fully characterized by standard analytical methods including matrix assisted laser desorption ionization time of flight (MALDI TOF) spectrometry, ¹H NMR spectroscopy, and UV-vis spectrometry, as illustrated by the vancomycin-associated protons in the ¹H NMR spectral data and by the strong UV absorption features that are consistent with vancomycin (λₘₐₓ=282 nm; ε=6716 M⁻¹cm⁻¹). In addition to MALDI measurement, selected members of the vancomycin conjugates were also characterized by using gel permeation chromatography (GPC) in order to measure their MWs and to determine the size distribution (Table 1). As summarized, the two sets of MWs, each determined independently by either MALDI or GPC, show similar MWs with generally narrow standard deviations lying within ±3 to 14% of their mean value. The vancomycin valency determined for each dendrimer conjugate is reported on a mean basis. These values were calculated by the analysis of the UV-vis absorptivity at 282 nm, but their determination by the alternative NMR method (see, e.g., Mullen, D.; et al., Chem. Eur. J. 2010, 16, 10675-10678; Mullen, D. G.; et al., ACS Nano 2010, 4, 657-670; Mullen, D. G.; Bioconjugate Chem. 2008, 19, 1748-1752; each herein incorporated by reference in its entirety)) was not attempted because of line broadening, signal overlaps, and unpredictable shifts of the vancomycin proton signals. The efficiency for vancomycin conjugation (([V]_{attach}÷[V]_{added})×100(%); V=vancomycin) was in the range of 70-100%. A greater conjugation efficiency (>92%) was observed with the lower-valent conjugates I and II. Such trends might be attributable in part to the influence of steric congestion on the reactions occurring on the dendrimer surface (see, e.g., Choi, S. K.; et al., Macromolecules 2011, 44, 4026-4029; herein incorporated by reference in its entirety)), which should be more serious as more molecules are conjugated (see, e.g., Tomalia, D. A.; et al., Polymer J. 1985, 17, 117-132; Tomalia, D. A.; et al., Angew. Chem., Int. Ed. Engl. 1990, 29, 138-175; each herein incorporated by reference in its entirety)).

TABLE 1
Selected macromolecular properties of PAMAM dendrimers
conjugated with vancomycin (V) molecules

| | | MW | | Valency |
|---|---|---|---|---|
| ID | Dendrimer-(V)ₙ | (g/mol)^{a} | MW_{w}^{b} (PDI^{c}) | (n)^{d} |
| I | Ac-G5-(V)_{1.2} | 31100 | 37800 (1.067) | 1 |
| II | Ac-G5-(V)_{2.3} | 32400 | 37600 (1.628) | 2 |
| III | Ac-G5-(V)_{3.5} | 32300 | 30700 (1.043) | 4 |
| IV | Ac-G5-(V)_{5.8} | 36300 | 37800 (1.027) | 6 |
| V | Ac-G5-(V)_{8.3} | 37500 | 33200 (1.032) | 8 |
| VI | GA-G5-(V)_{6.0} | 37100 | — | 6 |
| VII | DTPA-G5-(V)_{6.1} | — | 62500 (1.088) | 6 |
| VIII | DTPA-G5-(V)_{6.1}-(Fl)_{3.9} | — | — | 6 |
| IX | Ac-G5-(V)_{6.3}-(FITC)_{1.8} | 36300 | — | 6 |

^{a}Measured by matrix assisted laser desorption ionization (MALDI) mass spectrometry
^{b}Weight-averaged molecular weight determined by gel permeation chromatography (GPC)
^{c}Polydispersity index (PDI) = MW_{w} ÷ MWₙ
^{d}Refers to number of vancomycin molecules attached to a dendrimer molecule determined by UV-vis spectrometry; each number calculated on a mean basis and rounded to the nearest whole number

### Example 3

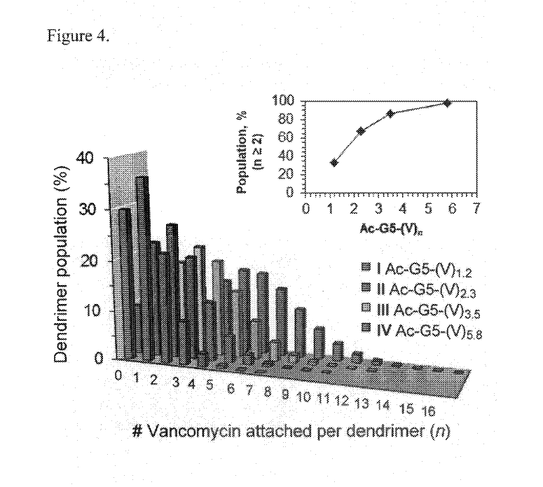

This example describes distribution of vancomycin valency. Currently only a few specialized methods are demonstrated for the engineering and functionalization of NPs with a precise number of ligands or drugs, such as those for PAMAM dendrimer (see, e.g., Mullen, D.; et al., Chem. Eur. J. 2010, 16, 10675-10678; Mullen, D. G.; et al., ACS Nano 2010, 4, 657-670; Mullen, D. G.; et al., Bioconjugate Chem. 2008, 19, 1748-1752; each herein incorporated by reference in its entirety)), polymer (see, e.g., Gu, F.; et al., Proc. Natl. Acad. Sci. U.S.A. 2008, 105, 2586-2591; herein incorporated by reference in its entirety)), or gold (see, e.g., Zhu, Y.; et al., Angew. Chem. Intl. Ed. 2010, 49, 1295-1298; Qian, H.; et al., Nano Lett. 2009, 9, 4083-4087; each herein incorporated by reference in its entirety)). Otherwise all NP conjugation reactions, including the amide coupling reaction used for the current conjugate synthesis, occur with a stochastic mechanism that leads to a distribution of conjugate populations comprised of a variable range of ligand and drug valencies (see, e.g., Mullen, D. G.; et al., ACS Nano 2010, 4, 657-670; Mullen, D. G.; et al., Bioconjugate Chem. 2008, 19, 1748-1752; each herein incorporated by reference in its entirety)). Therefore, a method for describing such dendrimer distributions differs from the way the mean valency (n) is determined and reported for each conjugate. FIG. 4 shows the distribution of the dendrimers simulated for each of the conjugates I-IV, Ac-G5-(V)ₙ, according to a Poissonian simulation (see, e.g., Mullen, D. G.; et al., ACS Nano 2010, 4, 657-670; Mullen, D. G.; et al., Bioconjugate Chem. 2008, 19, 1748-1752; each herein incorporated by reference in its entirety)). As an illustration, I Ac-G5-(V)ₙ (n=1.2) has approximately a valency of one vancomycin on an average basis but has a wider distribution including multivalent species (n =0-7; median of valency=4). Significantly, its populations of multivalent species (n≧2) add up to ˜34% (inset), suggesting that conjugate I does not entirely represent a monovalent species.

### Example 4

This example shows SPR spectroscopy for multivalent vancomycin interaction. SPR spectroscopy is a real-time kinetic method that allows the measurement of the on-rate constant (kₒₙ), the off-rate constant (k_{off}), and equilibrium dissociation constant K_{D} (=k_{off}/kₒₙ) for receptor-ligand interactions occurring on the surface (see, e.g., Rao, J.; et al., Chem. Biol. (Cambridge, Mass., U.S.) 1999, 6, 353-359; Rao, J.; et al., J. Am. Chem. Soc. 1999, 121, 2629-2630; Hong, S.; et al., Chem. Biol. (Cambridge, Mass., U.S.) 2007, 14, 107-115; Li, M.-H.; Eur. J. Med. Chem. 2012, 47, 560-572; Plantinga, A.; et al., ACS Med. Chem. Lett. 2011, 2, 363-367; Cooper, M. A.; et al., Bioorg. Med. Chem. 2000, 8, 2609-2616; each herein incorporated by reference in its entirety)). The experiments employed SPR spectroscopy to determine the binding constant K_{D} for dendrimer-based multivalent vancomycin conjugates to the cell wall ligands immobilized on the chip surface as a model for the bacterial cell surface. The model surface was prepared by utilizing each CMS sensor chip which was treated to present N^{α}-Ac-Lys-(D)-Ala-(D)-Ala or N^{α}-Ac-(D)-Ala-(D)-Lac as the cell wall precursor on the surface, each representing a vancomycin-susceptible and vancomycin-resistant cell wall model, respectively (see, e.g., Rao, J.; et al., Chem. Biol. (Cambridge, Mass., U.S.) 1999, 6, 353-359; Rao, J.; et al., J. Am. Chem. Soc. 1999, 121, 2629-2630; each herein incorporated by reference in its entirety)). Such a peptide-presenting chip was prepared typically following a N-(3-dimethylaminopropyl)-N′-ethylcarbodiimide (EDC)-based amide coupling method at a surface peptide density of 0.12 ng/mm² (equivalent to 2.2×10¹¹ molecules/mm²) (see, e.g., Hong, S.; et al., Chem. Biol. (Cambridge, Mass., U.S.) 2007, 14, 107-115; Plantinga, A.; et al., ACS Med. Chem. Lett. 2011, 2, 363-367; each herein incorporated by reference in its entirety)).

### Example 5

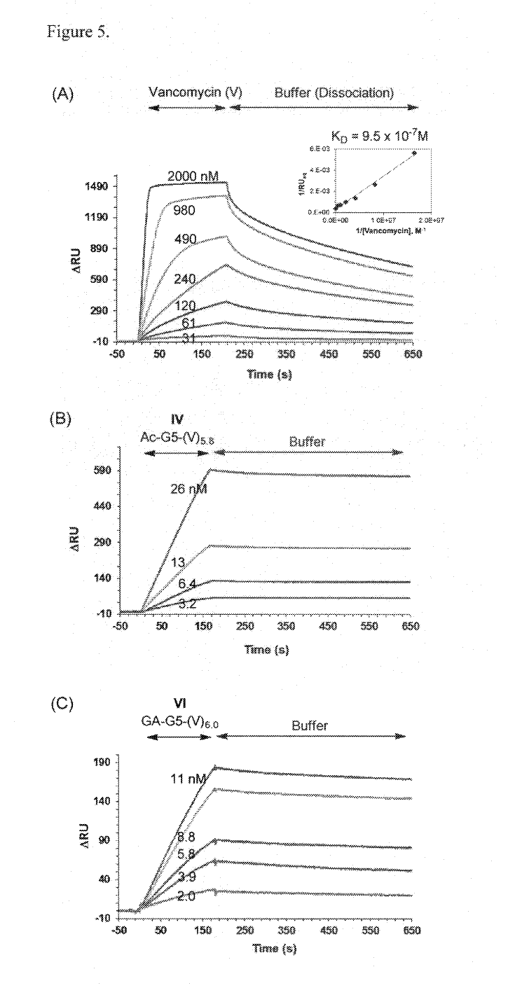

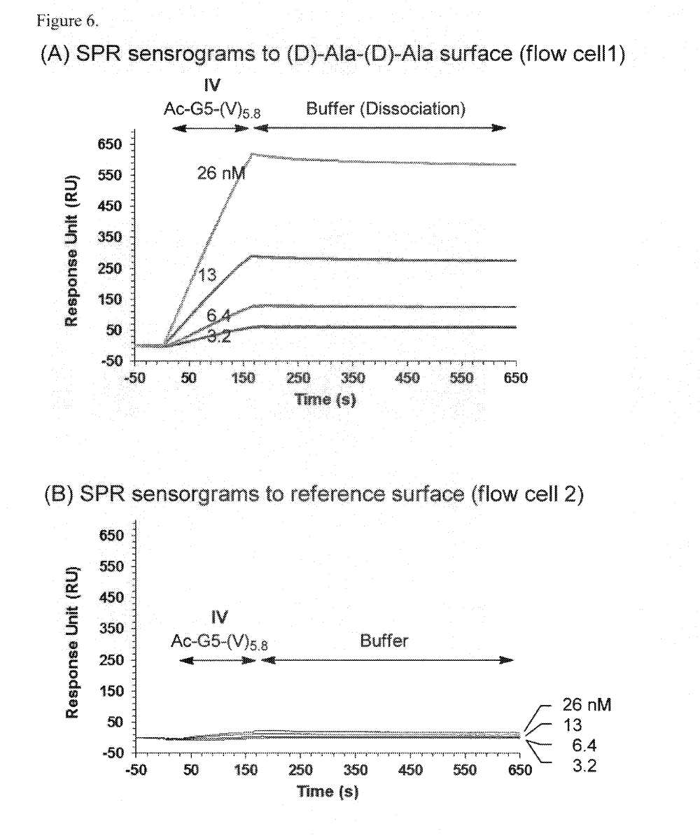

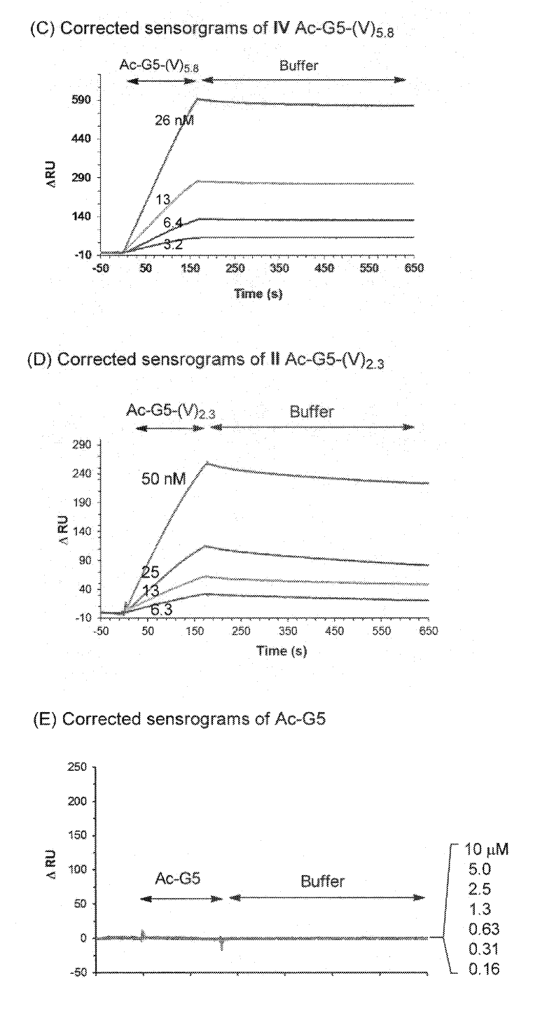

This example describes a vancomycin-susceptible cell wall model. Dose-dependent binding sensorgrams for vancomycin to the (D)-Ala-(D)-Ala surface are shown in FIG. 5A. Scatchard analysis of the SPR binding data provides a K_{D} value of 9.5×10⁻⁷ M, an affinity close to the value found in the literature (K_{D}≈10⁻⁶ M) (see, e.g., Rao, J.; et al., J. Am. Chem. Soc. 1999, 121, 2629-2630; herein incorporated by reference in its entirety)), demonstrating the susceptibility of the synthetic cell wall ligands to vancomycin binding. A fully acetylated G5 PAMAM, a negative control dendrimer without vancomycin attached, was tested for its binding but did not show any response to the (D)-Ala-(D)-Ala surface when otherwise measured under comparable conditions (see, FIG. 6). These data are supportive of the specificity of the cell wall model to vancomycin.

SPR binding studies were then performed for three representative vancomycin conjugates—II Ac-G5-(V)_{2.3}, IV Ac-G5-(V)_{5.8}, and VI GA-G5-(V)_{6.0}—each selected to address the effects of vancomycin valency and of dendrimer surface charge (see, FIGS. 5 and 6). First, the SPR sensorgrams obtained for each conjugate illustrate the concentration-dependent binding kinetics at the range of concentrations as low as 2 nM at which free vancomycin shows no detectable response. The results for each conjugate II, IV and VI suggest almost no evidence for non-specific binding by the conjugates as illustrated by conjugate IV. Conjugate IV binds to the surface of flow cell 1, the bacterial cell model that presents (D)-Ala-(D)-Ala peptide precursors (specific binding), but does not bind to the surface of flow cell 2, the reference surface that does not present this cell wall peptide (non-specific binding; see, FIG. 6). The result suggests, for example, binding specificity of IV to this cell wall model. Each sensorgram acquired by conjugate IV shows binding kinetics characterized by an extremely slow dissociation-rate (almost permanently bound)—a hallmark of tight multivalent binding, as reported in other vancomycin-based multivalent systems (see, e.g., Metallo, S. J.; et al., J. Am. Chem. Soc. 2003, 125, 4534-4540; Rao, J.; et al., Chem. Biol. (Cambridge, Mass., U.S.) 1999, 6, 353-359; Rao, J.; et al., J. Am. Chem. Soc. 1999, 121, 2629-2630; each herein incorporated by reference in its entirety)).

In order to determine the dissociation constant K_{D} for IV, each sensorgram was analyzed by a fitting analysis based on the Langmuir binding isotherm (see, e.g., Ober, R. J.; et al., Anal. Biochem. 2003, 312, 57-65; herein incorporated by reference in its entirety)). Despite the high level of uncertainty associated with such extremely slow dissociation, it was possible to extract estimated values for its k_{off} (≦4.4×10⁵ s⁻¹) and kₒₙ (4.5×10⁻⁵ M⁻¹s⁻¹) and, as a result, its K_{D} value (≦2.5×10⁻¹⁰ M). SPR sensorgrams were also analyzed for two other conjugates, II and VI, in a similar manner. Each of these conjugates showed a (sub)nanomolar K_{D} value as summarized in Table 2. The K_{D} values determined for II, IV, and VI suggest their binding avidity enhanced by a factor of 280-3800 (multivalent binding enhancement β=K_{D}^{mono}÷K_{D}^{multi}) relative to the free vancomycin molecule (K_{D}=950 nM). Even the divalent conjugate 2 Ac-G5-(V)₂ shows a K_{D} value of 3.4 nM, an avidity value that still provides a β value of 280 over vancomycin. The avidity values between the two conjugates IV and VI was also compared, each having the same mean valency of vancomycin but presented on a different dendrimer surface, either neutral or negatively charged surface, respectively. Only a slight difference was observed, suggesting, for example, that the avidity may have already reached a maximal level at a lower valency (cf., II), and/or the effect of the surface charge might be minimal.

TABLE 2
Rate constants and equilibrium dissociation constants K_{D} for binding kinetics of
G5-(V)ₙ to the bacterial cell wall model as determined by surface plasmon resonance (SPR)
spectroscopy. The model surface includes the vancomycin-susceptible, and vancomycin-
resistant model, each prepared by immobilization of either the N^{α}-Ac-Lys-(D)-Ala-(D)-Ala
peptide or N^{α}-Ac-Lys-(D)-Ala-(D)-Lac peptide on the surface of the sensor chip.

| | | | |
|---|---|---|---|
| kₒₙ (M⁻¹s⁻¹) | k_{off} (s⁻¹) | K_{D} (M)^{a} | β^{c,d} |

(D)-Ala-(D)-Ala surface (vancomycin-susceptible cell wall model)

| | | | | |
|---|---|---|---|---|
| Vancomycin | — | — | ^{b}9.5 × 10⁻⁷ (1.1 × | 1 |
| | | | 10⁻⁶)²⁵ | |
| II Ac-G5-(V)_{2.3} | 1.5 (±1.1) × 10⁵ | 4.5 (±2.6) × 10⁻⁴ | 3.4 (±1.9) × 10⁻⁹ | 280 (122) |
| IV Ac-G5-(V)_{5.8} | 4.5 × 10⁵ | 4.4 (±1.3) × 10⁻⁵ | 2.5 (±2.0) × 10⁻¹⁰ | 3800 (655) |
| VI GA-G5-(V)_{6.0} | 6.7 (±3.7) × 10⁵ | 3.6 (±1.9) × 10⁻⁴ | 5.4 (±0.6) × 10⁻¹⁰ | 1759 (293) |

(D)-Ala-(D)-Lac surface (vancomycin-resistant cell wall model)

| | | | | |
|---|---|---|---|---|
| Vancomycin | — | — | ^{b}1.5 × 10⁻³ (1.7 × 10⁻³)²⁴ | 1 |
| I Ac-G5-(V)_{1.2} | 9.8 (±7.1) × 10⁴ | 6.6 (±5.1) × 10⁻⁴ | 7.1 (±2.7) × 10⁻⁹ | 2.1 × 10⁵ (1.8 × 10⁴) |
| II Ac-G5-(V)_{2.3} | 3.2 × 10⁵ | 4.1 (±1.3) × 10⁻⁴ | 8.1 × 10⁻⁹ | 1.9 × 10⁵ (8.1 × 10⁴) |
| III Ac-G5-(V)_{3.5} | 6.4 × 10⁵ | 4.5 (±2.7) × 10⁻⁴ | 2.0 (±1.8) × 10⁻⁹ | 7.5 × 10⁵ (2.1 × 10⁵) |
| IV Ac-G5-(V)_{5.8} | 6.9 × 10⁵ | 2.9 (±0.4) × 10⁻⁴ | 1.7 × 10⁻⁹ | 8.8 × 10⁵ (1.5 × 10⁵) |
| VI GA-G5-(V)_{6.0} | 1.8 (±1.7) × 10⁶ | 2.8 (±1.5) × 10⁻³ | 3.4 × 10⁻⁹ | 4.4 × 10⁵ (7.4 × 10⁴) |

^{a}Each dissociation constant K_{D} (= k_{off}/kₒₙ) is not derived directly from the pair of mean k_{off} and kₒₙ values as given in the table. Rather, it represents a mean value calculated by averaging a set of K_{D} values, each calculated from an individual pair of k_{off} and kₒₙ values determined per injection concentration. At least four different concentrations were used for the calculation, each run in duplicate per concentration. The number within parentheses refers to the standard error of the mean (SEM), and unless noted specifically, the standard error for each K_{D} value is within 2-fold variation.
^{b}Determined by Scatchard analysis
^{c}β = Multivalent binding enhancement = K_{D}^{vancomycin} ÷ K_{D}^{G5-(V)n} where K_{D}^{vancomycin} and K_{D}^{G5-(V)n} refer to the dissociation constants determined for vancomycin and G5-(V)ₙ conjugates I-IV and VI, respectively.
^{d}The value within parentheses refers to the valency-corrected value (=β ÷ n)

The SPR analysis shows that such improved avidity by each conjugate is attributed primarily to the slower off-rate (k_{off}). This observation fully supports the established hypothesis that complete dissociation of a multivalent species occurs very slowly because all of the individual binding interactions have to dissociate simultaneously from the surface (see, e.g., Arranz-Plaza, E.; et al., J. Am. Chem. Soc. 2002, 124, 13035-13046; Adler, P.; et al., J. Biol. Chem. 1995, 270, 5164-5171; Mammen, M.; et al., Angew. Chem. Int. Ed. 1998, 37, 2754-2794; each herein incorporated by reference in its entirety)). Altogether, the SPR study performed on the vancomycin-susceptible cell wall model suggests, for example, that the avidity by the vancomycin conjugates is enhanced by two to three orders of magnitude, relative to the micromolar affinity of free vancomycin. It validates, for example, the hypothesis of targeting bacterial cells by using a vancomycin-presenting dendrimer platform.

### Example 6

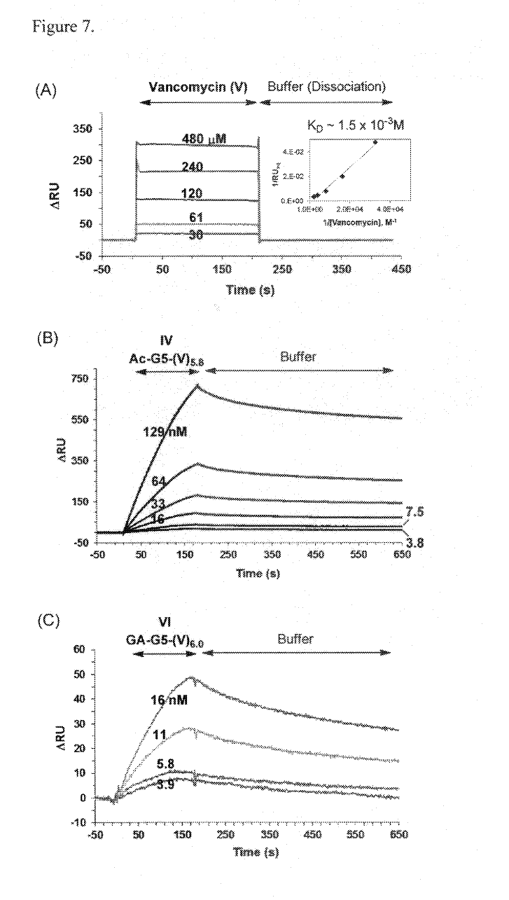

This example describes a vancomycin-resistant cell wall model. The SPR study was extended to examine another cell wall model that mimics the vancomycin-resistant bacterial cell. In this model, (D)-Ala-(D)-Lac peptides are immobilized in lieu of the (D)-Ala-(D)-Ala residue as the cell wall precursor, and the resulting surface shows reportedly ˜1000-fold reduction in affinity to free vancomycin (K_{D}≈10⁻³M) (see, e.g., Rao, J.; et al., Chem. Biol. (Cambridge, Mass., U.S.) 1999, 6, 353-359; Walsh, C. T.; et al., Chem. Biol. (Cambridge, Mass., U.S.) 1996, 3, 21-28; each herein incorporated by reference in its entirety)). Dose-dependent binding experiments were performed for vancomycin to the (D)-Ala-(D)-Lac surface as shown in FIG. 7A. In contrast to the susceptible model surface, vancomycin shows very low responses, even when injected at much higher concentrations. The Scatchard analysis (inset) of its binding responses provides a K_{D} estimate of 1.5×10⁻³M, a value close to the value in the literature (K_{D}=1.7×10⁻³M) (see, e.g., Rao, J.; et al., Chem. Biol. (Cambridge, Mass., U.S.) 1999, 6, 353-359; herein incorporated by reference in its entirety)). This control experiment confirmed that this chip mimics the vancomycin-resistant cell wall model by showing only millimolar affinity to free vancomycin.

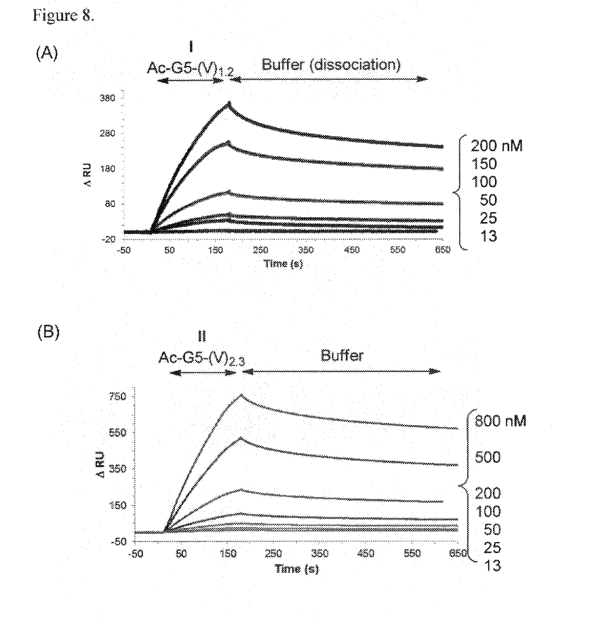

Whether multivalent ligand presentation enables the generation of a high avidity species that binds tightly to the vancomycin-resistant surface and improves the poor affinity of free vancomycin was next investigated (see, e.g., Hong, S.; et al., Chem. Biol. (Cambridge, Mass., U.S.) 2007, 14, 107-115; Mammen, M.; et al., Angew. Chem. Int. Ed. 1998, 37, 2754-2794; Lee, Y. C.; et al., Acc. Chem. Res. 1995, 28, 321-327; Kiessling, L. L.; et al., Curr. Opin. Chem. Biol. 2000, 4, 696-703; each herein incorporated by reference in its entirety)). SPR experiments were performed for a series of vancomycin conjugates, I-IV Ac-G5-(V)ₙ and VI GA-G5-(V)₆, each selected to cover a range of valencies and to address the difference in surface charge (see, FIGS. 7 and 8). The experiments showed remarkably strong binding responses to the surface in a concentration-dependent manner, even at low nanomolar concentrations. Compared to the vancomycin-sensitive surface (FIG. 5), the dissociation kinetics of the higher-valent conjugates IV and VI is characterized by slightly faster dissociation (k_{off}) by approximately an order of magnitude (Table 2). Despite such differences, each of these conjugates still showed a K_{D} value in the low nanomolar range (K_{D}=˜2-3 nM). This avidity constant represents an enhancement by more than five orders of magnitude relative to the millimolar affinity of the free vancomycin molecule. The lower-valent conjugates I and II also showed K_{D} values of 7-8 nM, which is slightly lower in avidity than those of the higher-valent conjugates,

Given the low valency (n=1.2) of conjugate I Ac-G5-(V)_{1.2}, its K_{D} value is still remarkably low at the nanomolar range and its avidity enhancement is comparable to that displayed by each higher-valent conjugate, II-VI. It was hypothesized that such a high avidity constant exhibited by I is not due to tight binding by its monovalent conjugate but reflects that of the distribution of its population. As discussed earlier in the Poissonian distribution of I (FIG. 4), only 36% of the dendrimer species in this sample represent the monovalent dendrimer (n=1). Approximately the same fraction (34%) of the dendrimer populations belongs to the multivalent species (n≧2), and such a fraction might contribute to the binding responses observed for I.

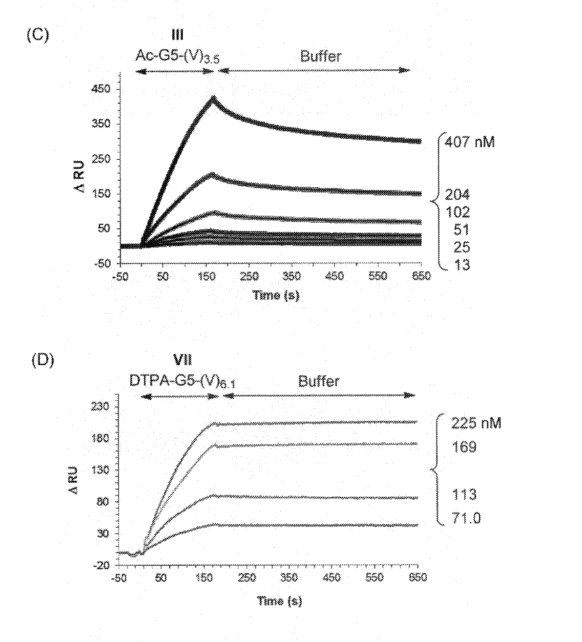

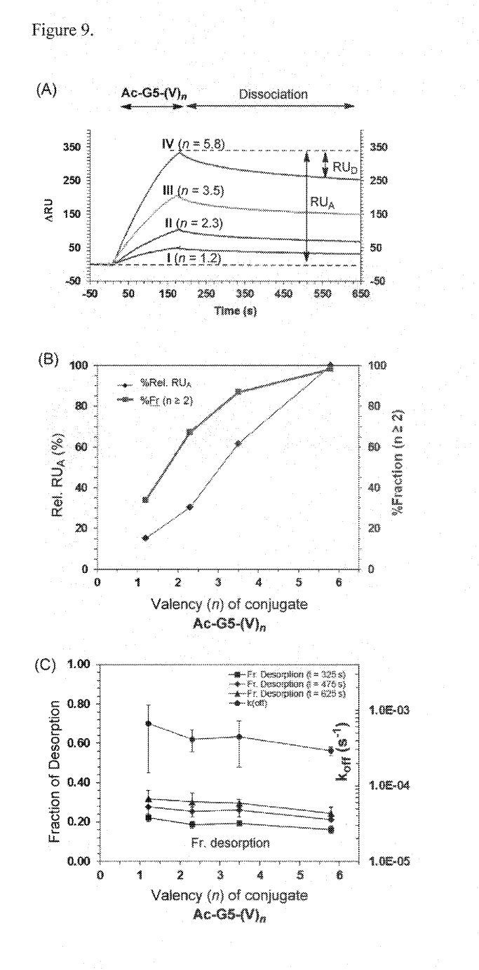

In order to better understand the binding kinetics displayed by the current stochastically-prepared multivalent dendrimers, each sensorgram (adsorption, desorption) was analyzed by fractional analysis for conjugates I-IV Ac-G5-(V)ₙ, each measured at the identical concentration 50 nM (FIG. 9). First the maximal level of adsorption (RU_{A}) observed by each conjugate is ordered as follows: IV>III>II>I (FIG. 5B). Such differences in adsorption are not simply explained by their equilibrium association constants (K_{A}=K_{D}⁻¹: IV≈III>II≈I). Interestingly, it is better correlated with the fraction of multivalent species (n≧2) distributed in each conjugate (FIG. 4), suggesting that these multivalent species are significantly responsible for the adsorption to the surface. In contrast, the separate desorption analysis performed for each dissociation curve shows almost no difference in fractional desorption between the four sensorgrams. This analysis is illustrated by a plot of the off-rate constant (k_{off}, Table 2) and the level of fractional desorption (=RU_{D}/RU_{A}) for each conjugate (FIG. 9C). It suggests that, for example, those dendrimer populations bound at the end of each association phase are likely represented by tight binding species, each having a valency of n≧2. This fractional binding is supported by (sub)nanomolar dissociation constants associated with reported divalent and trivalent vancomycin species (see, e.g., Rao, J.; et al., Science (Washington, D.C., U.S.) 1998, 280, 708-711; Rao, J.; et al., Chem. Biol. (Cambridge, Mass., U.S.) 1999, 6, 353-359; Rao, J.; et al., J. Am. Chem. Soc. 1999, 121, 2629-2630; each herein incorporated by reference in its entirety)).

Another aspect to consider that is important for understanding the mechanistic basis of multivalent association is the effective molarity of surface ligands available for binding. Effective molarity (M_{eff}) or effective concentration (see, e.g., Page, M. I.; et al., Proc. Natl. Acad. Sci. U.S.A. 1971, 68, 1678-1683; herein incorporated by reference in its entirety)) in the analysis of intramolecular catalysis—refers to the ratio of the equilibrium constant of an intramolecular association to that of an analogous intermolecular association (see, e.g., Rao, J.; et al., J. Am. Chem. Soc. 1997, 119, 10286-10290; Mackay, J. P.; et al., J. Am. Chem. Soc. 1994, 116, 4581-4590; Hunter, C. A.; et al., Angew. Chem. Intl. Ed. 2009, 48, 7488-7499; Krishnamurthy, V. M.; et al., J. Am. Chem. Soc. 2007, 129, 1312-1320; each herein incorporated by reference in its entirety)). In the system, M_{eff} as [(K_{D}^{vancomycin})×(K_{D}^{vancomycin})]/[(K_{D1}^{G5-(V)n})×(K_{D2}^{G5-(V)n})]≈(K_{D}^{vancomycin})²/(K_{D}^{G5-(V)n}) (see, Rao, J.; et al., J. Am. Chem. Soc. 1997, 119, 10286-10290; Adler, P.; et al., J. Biol. Chem. 1995, 270, 5164-5171; Page, M. I.; et al., Proc. Natl. Acad. Sci. U.S.A. 1971, 68, 1678-1683; Mackay, J. P.; et al., J. Am. Chem. Soc. 1994, 116, 4581-4590; each herein incorporated by reference in its entirety)) where K_{D1}^{G5-(V)n} and K_{D2}^{G5-(V)n} refer to the first and second dissociation constant of a divalently-bound conjugate G5-(V)ₙ, respectively (for definition of K_{D}^{vancomycin} and K_{D}^{G5-(V)n, see Table} 2 footnotes). M_{eff} is characterized as an effective local concentration of surface ligands that contribute to the second binding event. Values of M_{eff} calculated for conjugates II, IV and VI are ˜2.7×10⁻⁴, 3.6×10⁻³ and 1.7×10⁻³ M, respectively, when applied for their adsorption to the vancomycin-susceptible surface presenting (D)-Ala-(D)-Ala ligand molecules. This analysis indicates that the M_{eff} values of the peptide ligand are two to three orders of magnitude higher than the dissociation constant of the same ligand to vancomycin (K_{D}=9.5×10⁻⁷ M). It was assumed that such difference is large enough to explain the tight adsorption of these conjugates. M_{eff} values were estimated for the other vancomycin-resistant surface that presents (D)-Ala-(D)-Lac ligand molecules. The corresponding values of M_{eff} for conjugates I-VI lied in the range of ˜280 to 1300 M. Such ligand molarities are implausibly high, but support the high avidity adsorption of the conjugates to the drug-resistant surface because the ligand concentrations are extremely high and far above the dissociation constant of vancomycin to the same ligand (K_{D}≈1.5×10⁻³ M).

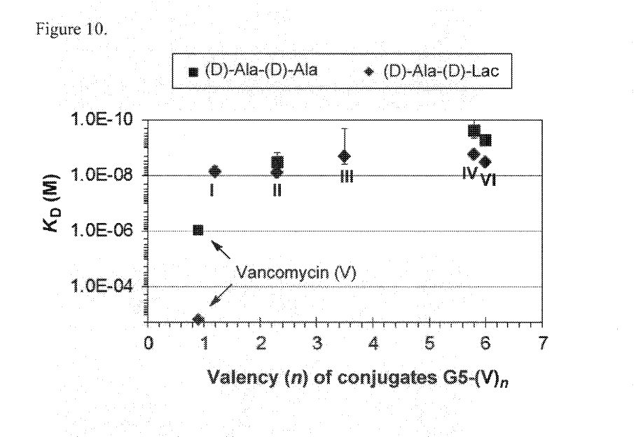

In summary, the SPR study was performed, for example, to determine the equilibrium dissociation constants K_{D} of G5-(V)ₙ I-IV and VI to the cell wall model made of either (D)-Ala-(D)-Ala or (D)-Ala-(D)-Lac peptide precursor, as summarized in FIG. 10. This study demonstrated the effectiveness of the multivalent strategy for achieving high avidity binding to the cell wall models, including the vancomycin-resistant surface, and therefore suggests its capability for effective targeting of bacterial cells.

### Example 7

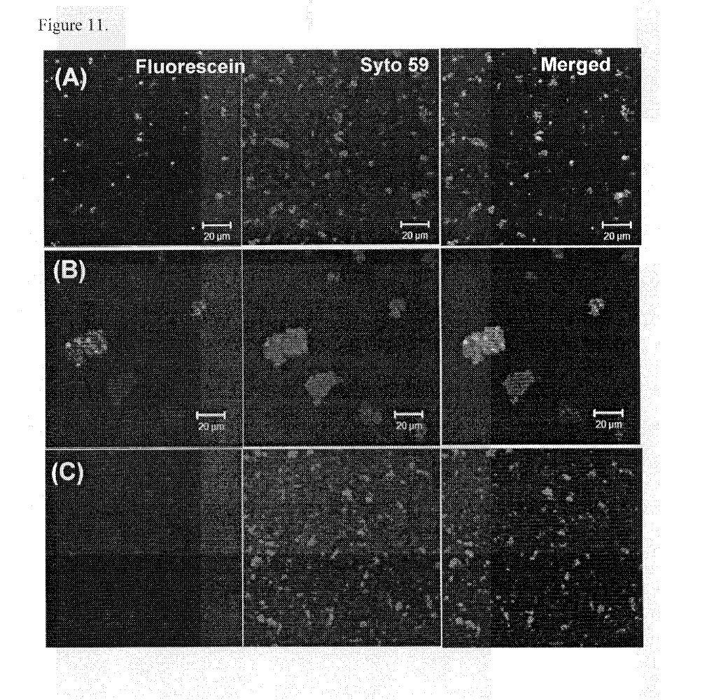

This example describes confocal microscopy experiments. Confocal microscopy was next performed to determine whether the SPR binding study for cell wall models is translatable to bacterial cells (FIG. 11). Gram-positive bacteria Staphylococcus aureus were treated with fluorescein-labeled vancomycin conjugates VIII DTPA-G5-(V)_{6.1}-(Fl)_{3.9} and IX Ac-G5-(V)_{6.3}-(FITC)_{1.8}, as shown in FIG. 11. The treatment resulted in punctate green fluorescence (FIG. 11A, B). Since this green fluorescence comes from the dye on the conjugate, each image indicates binding of the dendrimer to the cell surface. In contrast, cells treated with a non-targeted control dendrimer, GA-G5-(FITC) (FIG. 11C) or Ac-G5-(FITC), showed no noticeable green fluorescence on the cells. Each sample of the treated cells was also stained for DNA using Syto®59, which confirmed that the green spots associated with the dendrimers were in fact associated with intact cells rather than cellular debris. Interestingly, treatment of the cells with IX Ac-G5-(V)_{6.3}-(FITC)_{1.8} resulted in large clumps or aggregates of bacterial cells. This is most likely due, for example, to the crosslinking of multiple bacterial cells, mediated by multiple vancomycin molecules on each dendrimer.

### Example 8

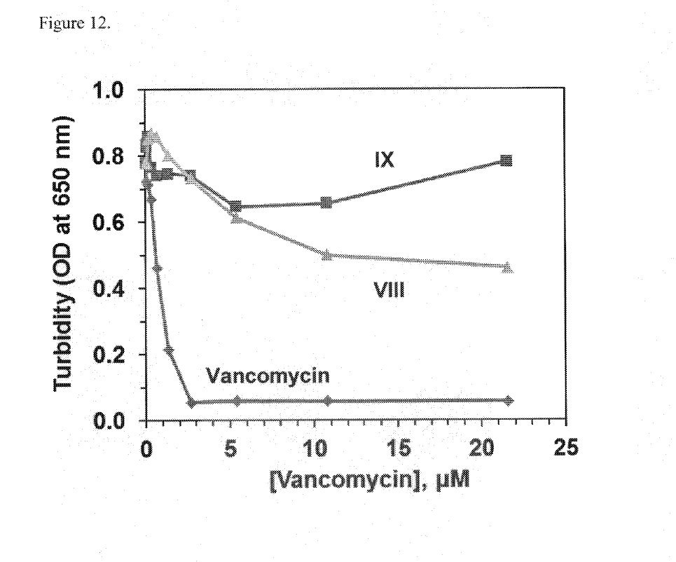

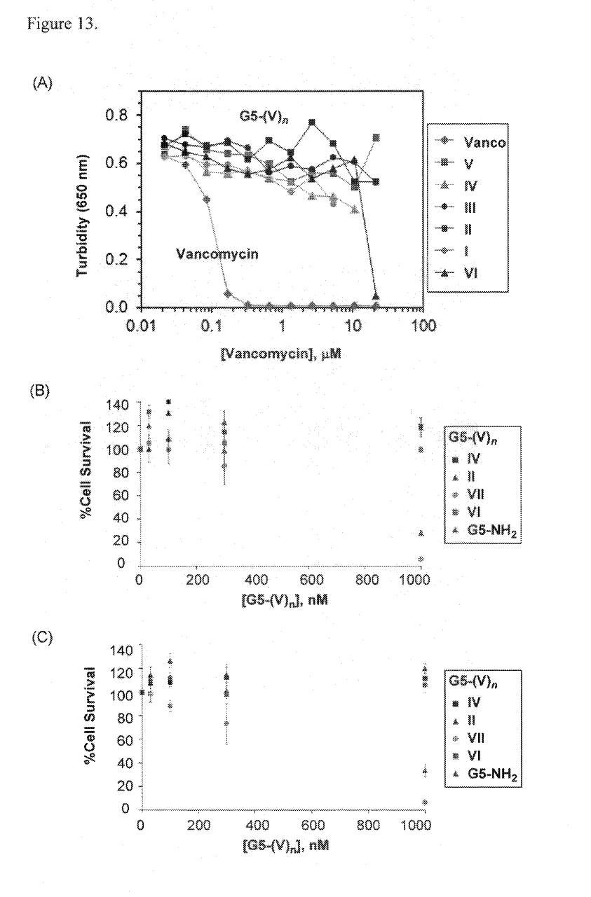

This example describes bacterial cell lysis experiments. Whether the binding of the vancomycin-conjugated dendrimer to the bacterial membrane causes bacterial cell lysis was next investigated. Cell lysis constitutes one of the mechanisms for killing bacteria (see, e.g., Chung, H. S.; et al., Proc. Natl. Acad. Sci. U.S.A. 2009, 106, 21872-21877; herein incorporated by reference in its entirety)), though lack of lysis does not necessarily preclude the therapeutic effectiveness of tested conjugates (see, e.g., Lunde, C. S.; et al., Antimicrob. Agents Chemother. 2009, 53, 3375-3383; herein incorporated by reference in its entirety)). On the other hand, the lack of such a mechanism would be more desirable for the diagnostic applications that aim for bacterial detection, isolation, and enumeration of whole cells. To determine the degree of cell lysis, a turbidity assay was employed. This assay quantitates the bacterial population in a culture by measuring the optical density (OD at 650 nm), and the cell populations are correlated with the degree of lysis (see, e.g., Chung, H. S.; et al., Proc. Natl. Acad. Sci. U.S.A. 2009, 106, 21872-21877; Lunde, C. S.; et al., Antimicrob. Agents Chemother. 2009, 53, 3375-3383; each herein incorporated by reference in its entirety)). Effects of the conjugates I-VI, VIII, IX on the cell growth rate were determined using the same Gram-positive strain Staphylococcus aureus and were presented as a function of the inhibitor concentration (FIGS. 12 and 13). As illustrated by conjugates VIII and IX in FIG. 12, the bacterial cultures exposed to the vancomycin conjugate showed much less change in optical density than free vancomycin, which decreased the optical density to the background level at ≧0.1 μM. However, certain differences in the lysis activity were also seen amongst the conjugates. Conjugate VIII shows a greater reduction in optical density than IX. Other conjugates tested I-VI showed no significant or only small changes in the turbidity assay at the high doses of ≦20 μM (FIG. 13A).

This assay result suggests, for example, that the vancomycin-conjugated dendrimer has low activity for causing bacterial cell lysis despite its adsorption to the cell membrane, as indicated by the confocal images. Such mode of activity is not unique to the current vancomycin-conjugated dendrimers but is also reported for vancomycin-derived glycopeptide antibiotics including telavancin (Vibativ™) that shows potent bactericidal activity without causing cell lysis (see, e.g, Long, D.; et al., J. Antibiot. 2008, 61, 603-614; Lunde, C. S.; et al., Antimicrob. Agents Chemother. 2009, 53, 3375-3383; each herein incorporated by reference in its entirety)). Given the complexities of the interactions between PAMAM dendrimers and bacterial membranes (see, e.g., Calabretta, M. K.; et al., Biomacromolecules 2007, 8, 1807-1811; herein incorporated by reference in its entirety)), there should be other factors that limit membrane penetration and cell lysis, such as the nanometer size of the conjugate particle (d≧5.4 nm) and the polyionic nature of the surface. Such lack of cell lysis provides, for example, a significant advantage for diagnostic purposes, as sensitive bacterial detection depends not only on its tight binding to the bacterial cell, but also on its ability to retain the intact cell for the entire duration of the assay.

### Example 9

This example describes mammalian cell toxicity experiments. In addition to the bacteria-targeted assays, the effect of vancomycin-conjugated dendrimers to mammalian cells was investigated (see, e.g., Leroueil, P. R.; et al., Acc. Chem. Res. 2007, 40, 335-342; Thomas, T. P.; et al., Biomacromolecules 2009, 10, 3207-3214; each herein incorporated by reference in its entirety)). Human cervical KB cells and mouse melanoma B16-F10 cells were separately studied to evaluate the cytotoxicity of the representative conjugates II, IX, VI, and VII (FIG. 13) (see, e.g., Thomas, T. P.; et al., Biomacromolecules 2009, 10, 3207-3214; herein incorporated by reference in its entirety)). As a reference, the positively charged G5-NH₂ showed a dose-dependent cytotoxicity apparently starting at 1000 nM, as reported earlier (see, e.g., Thomas, T. P.; et al., Biomacromolecules 2009, 10, 3207-3214; herein incorporated by reference in its entirety)). The vancomycin conjugates II, IV, and VI were not cytotoxic when tested under the otherwise identical conditions. Such a result, which is consistent with the relatively no cytotoxicity displayed by neutral or negatively-charged dendrimers, suggests that further modifications made through vancomycin conjugation did not lead to any effect on cell growth. However, conjugate 7, the surface of which is fully covered with metal-free DTPA groups, showed toxicity at 1000 nM as potent as G5-NH₂. Such growth inhibition is not understood at this time, but it was speculated that this effect might be related, for example, to the high local concentration of the free DTPA group ([DTPA]_{free}≈0.1 mM), a strong metal chelator which is linked to depletion of endogenous trace metals (see, e.g., Lauffer, R. B.; Chem. Rev. (Washington, D.C., U.S.) 1987, 87, 901-927; Byegård, J.; et al., J. Radioanal. Nucl. Chem. 1999, 241, 281-290; each herein incorporated by reference in its entirety)).

### Example 10

This example describes synthesis of dendrimer-coated IONP. After identification of the vancomycin-conjugated dendrimers that show high avidity to the bacterial surface, the applicability of this dendrimer platform as laboratory and clinical tools that enable selective binding and isolating of bacteria was investigated. Whether the bacteria-targeting technology can be combined with the speed and convenience provided by magnetic isolation technology was investigated. Two conjugates, VI GA-G5-(V)_{6.0} and VII DTPA-G5-(V)_{6.1}, were selected for this purpose, and each was coupled with an iron oxide nanoparticle (IONP) to generate magnetic nanodevices (Scheme 2). In the first step, IONP (Fe₃O₄; mean d<50 nm) was treated with (3-aminopropyl)trimethoxysilane in order to chemically modify its surface to present primary amines (IONP-NH₂) that will serve as the chemical handle for dendrimer conjugation (see, e.g., Laurent, S.; et al., Chem. Rev. (Washington, D.C., U.S.) 2008, 108, 2064-2110; herein incorporated by reference in its entirety)). Each conjugate, VI or VII, was covalently attached to the IONP-NH₂ by an EDC-based amide coupling method, yielding IONP-VI, and IONP-VII, respectively. Each of these dendrimer-coated IONPs was characterized by UV-vis spectrometry, confirming the presence of a vancomycin conjugate (Scheme 2; λₘₐₓ=282 nm). The conjugation efficiency of VI or VII to IONP was estimated by colorimetric analysis, which indicated that ˜75% of the conjugates added in the reaction mixture were covalently attached.

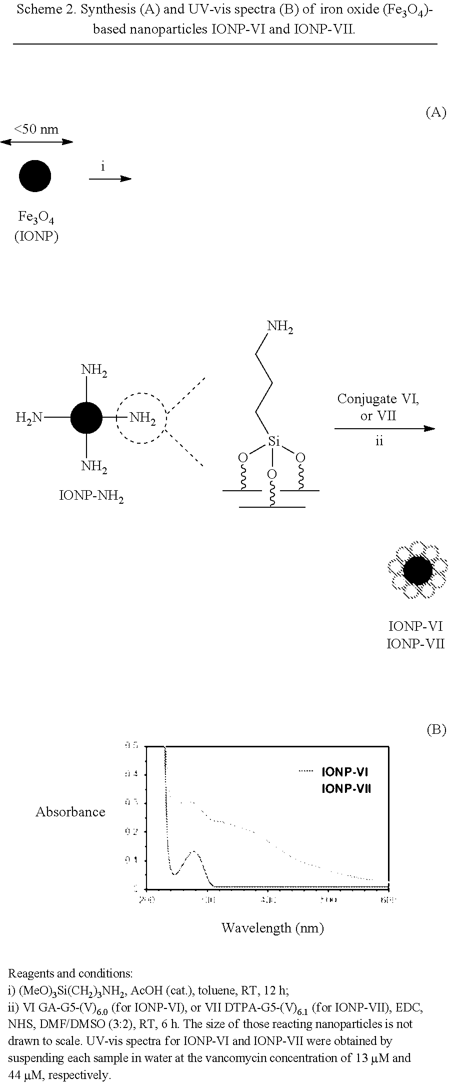

The above method developed for IONP fabrication with vancomycin-conjugated dendrimers was also applicable for the synthesis of other types of dendrimer-coated IONPs including those designed for targeting Gram negative bacteria. G5 PAMAM dendrimers conjugated with the polymyxin B ligand G5-(Polymyxin) were prepared to illustrate this purpose. However this method is also applicable for G5 PAMAM dendrimers conjugated with other types of ligands targeting Gram negative cells such as collistin (see, e.g., Shaheen, M, et al. Chem. Biol. (Cambridge, Mass., U.S.) 2011, 18, 1640-48), mannoside (see, e.g., Liang, M. N., et al. Proc. Natl. Acad. Sci. U.S.A. 2000, 97, 13092-96) and N-acetylneuraminic acid (sialic acid; see, e.g., Agnani, G., et al. Infection and Immunity 2003, 71, 991-96). Preparation of magnetic IONP nanoparticles targeting Gram negative bacteria is based on the coupling of G5-(Polymyxin) or G5-(Gram negative cell-targeting ligand) conjugate with IONP as illustrated in Scheme 2. The dendrimer coupling was achieved by covalent attachment of G5-(Polymyxin) to the IONP-NH₂ by an EDC-based amide method, yielding IONP-G5(Polymyxin). This dendrimer-coated IONP was characterized by UV-Vis spectrometry, confirming the presence of a polymyxin B conjugate (λₘₐₓ=285 nm). The conjugation efficiency of G5-(Polymyxin) to IONP was determined by UV-Vis colorimetric analysis, which indicated that ˜52-72% of the dendrimer conjugate added in the reaction mixture was covalently attached.

### Example 11

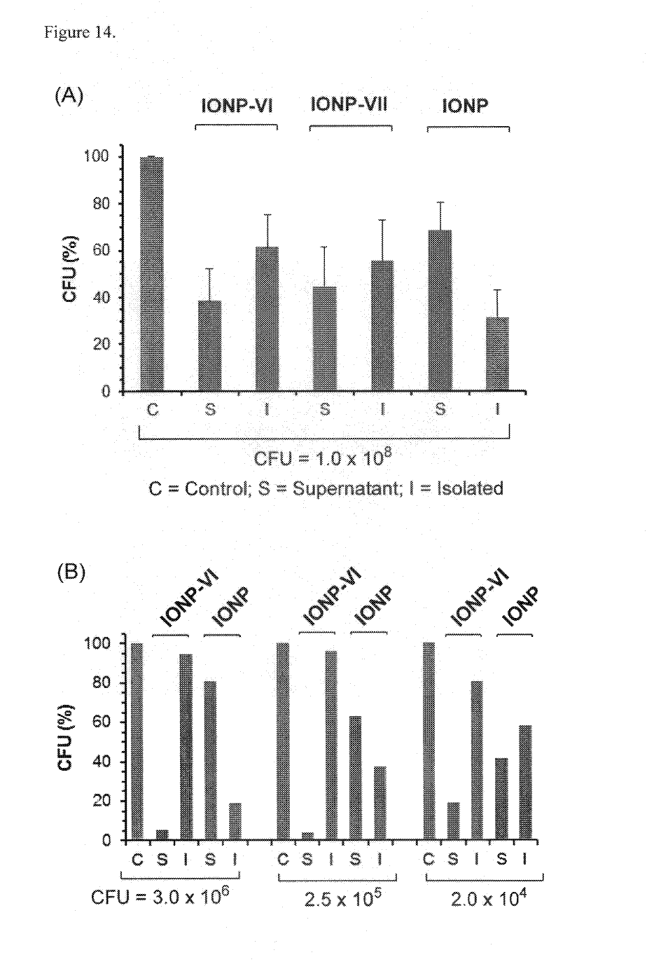

This example describes magnetic bacterial isolation. The effectiveness of bacterial removal in aqueous samples by bacteria-targeting magnetic nanoparticles was tested. The amount of bacteria was quantified both in the supernatant and on IONPs (see, e.g., Schmidt, M.; Transfusion Medicine and Hemotherapy 2011, 38, 259-265; herein incorporated by reference in its entirety)) where aliquots of the supernatant and isolated IONPs were serially diluted and plated on agar plates to enumerate bacterial CFU. FIG. 14 summarizes the results obtained by IONP-VI and IONP-VII for their ability to isolate Staphylococcus aureus bacterial cells from highly enriched bacterial samples (0.25 mg IONP per 10⁴-10⁸ CFU bacteria). The ratio of bacterial cells added per IONPs varied over four orders of magnitude in bacterial CFU and each of such bacterial titers was arbitrarily chosen to gauge the low range of the IONPs in the isolation of bacterial cells. Briefly, this procedure was performed in three steps: i) incubation of bacteria inoculum with the IONPs, ii) magnetic separation of bacteria adhered to IONPs from the supernatant, iii) enumeration of bacterial CFU of the pellet by using the agar culture method.

First, at the highest bacterial titer (10⁸ CFU; FIG. 9A), an unmodified IONP which is the non-targeted control showed about 69(±12)% of the bacteria remained in the supernatant relative to the level obtained by the IONP-free control, and therefore the bare IONP control showed the ability to capture ˜31% of the bacteria added. This result is largely consistent with the conclusion reached about unmodified iron oxide particles that is reported elsewhere that its ionic surface can promote non-specific adhesion of bacterial cells primarily through electrostatic interactions (see, e.g., Li, B.; et al., Colloids Surf, B 2004, 36, 81-90; Brown, G. E.; et al., Chem. Rev. (Washington, D.C., U.S.) 1998, 99, 77-174; each herein incorporated by reference in its entirety)). In comparison, targeted experiments that were performed using both IONP-VI and IONP-VII showed 61(±14)% and 56(±17)% of bacterial isolation, respectively. Thus, each of these dendrimer-coated IONPs was able to capture bacterial cells at the efficiency ˜2-fold greater than the non-targeted control. Such capturing capability appears to be compromised due to excess bacterial loads and indicates a maximal level of the bacteria to be captured per IONPs added.

Second, the efficiency of bacterial capture by IONP-VI and IONP at the lower range of bacterial titers (10⁴-10⁶ CFU; FIG. 14B) were compared. Targeted experiments using IONP-VI showed 81-96% of bacterial isolation, an efficiency greater than those obtained in the higher bacterial titer (FIG. 14A). Thus, the results support that the average level of bacterial capture is correlated with the bacterial load per targeted IONPs. Such results from the targeted IONP-VI are greater than those from the non-targeted IONP which showed approximately 20-60% bacterial isolation under the same conditions. Interestingly, the efficiency of the bare IONP rapidly deteriorated in response to the increase in bacterial load, suggesting that its capture capability is limited and easily saturable.

### Example 12

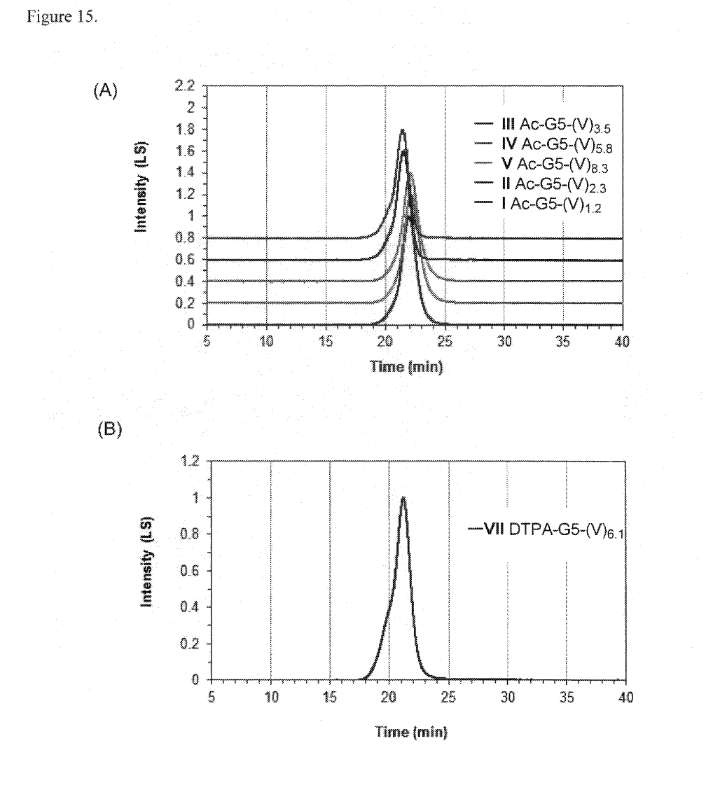

FIG. 15 shows GPC chromatograms of G5 PAMAM dendrimer conjugates, each linked with vancomycin (V) molecules at a variable ratio (n) of vancomycin to the dendrimer molecule. (A) I-V Ac-G5-(V)ₙ=1.2, 2.3, 3.5, 5.8, 8.3); (B) VII DTPA-G5-(V)_{6.1}.

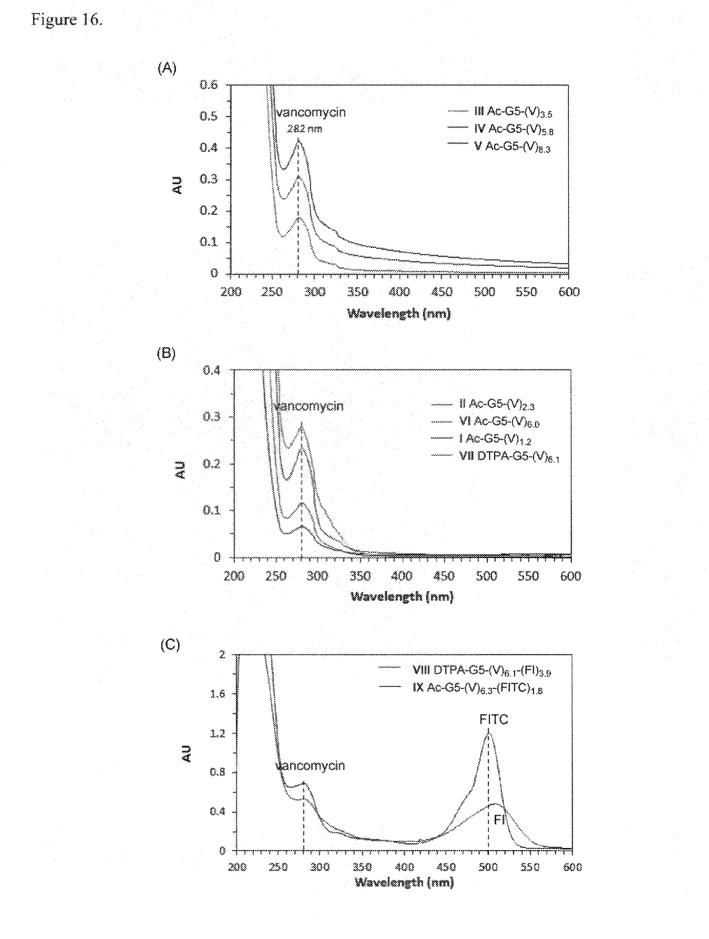

FIG. 16 shows UV-vis spectra of G5 dendrimer-vancomycin conjugates G5-(V)ₙ. Each of the dendrimer conjugates was measured in PBS (pH 7.4) at the concentration of the dendrimer as indicated. (A) III Ac-G5-(V)_{3.5} ([dendrimer]=7.0 μM), IV Ac-G5-(V)_{5.8} (6.5 μM), V Ac-G5-(V)_{8.3} (5.8 μM); (B) II Ac-G5-(V)_{2.3} (7.1 μM), VI GA-G5-(V)_{6.0} (5.2 μM), I Ac-G5-(V)_{1.2} (7.6 μM); VII DTPA-G5-(V)_{6.1} (4.2 μM); (C) VIII DTPA-G5-(V)_{6.1}-(Fl)_{3.9} (6.5 μM), IX Ac-G5-(V)_{6.3}-(FITC)_{1.8} (6.4 μM).

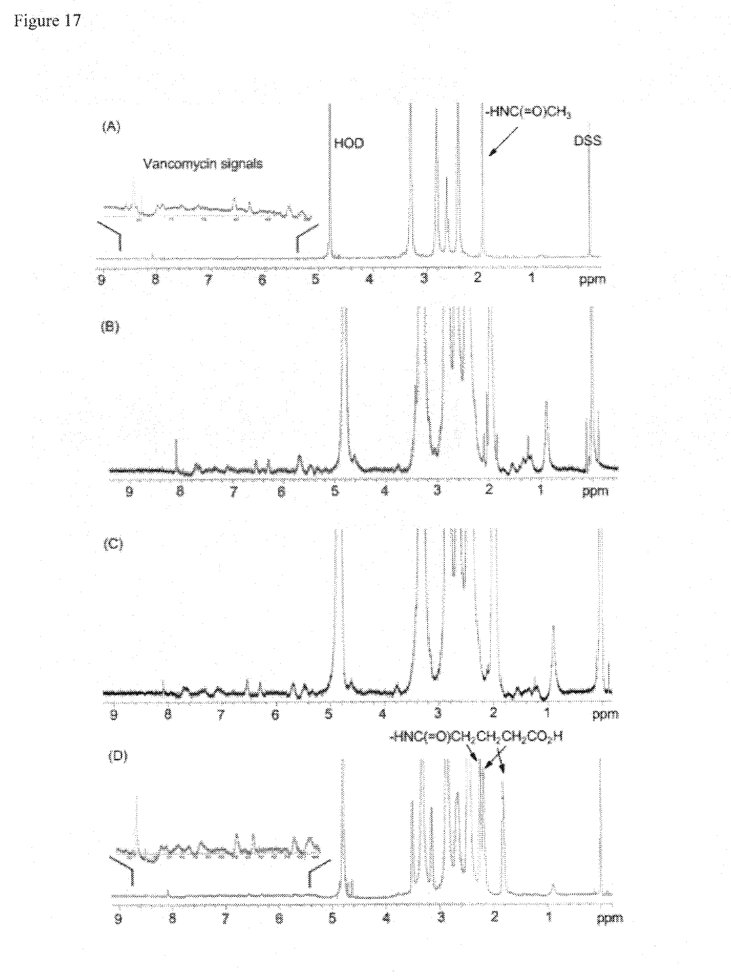

FIG. 17 shows selected ¹H NMR spectra of vancomycin-conjugated dendrimers G5-(V)ₙ. (A) II Ac-G5-(V)_{2.3}; (B) III Ac-G5-(V)_{3.5}; (C) IV Ac-G5-(V)_{5.8}, (D) VI GA-G5-(V)_{6.0}. Each NMR spectrum was acquired in D₂O (5 mg/mL).

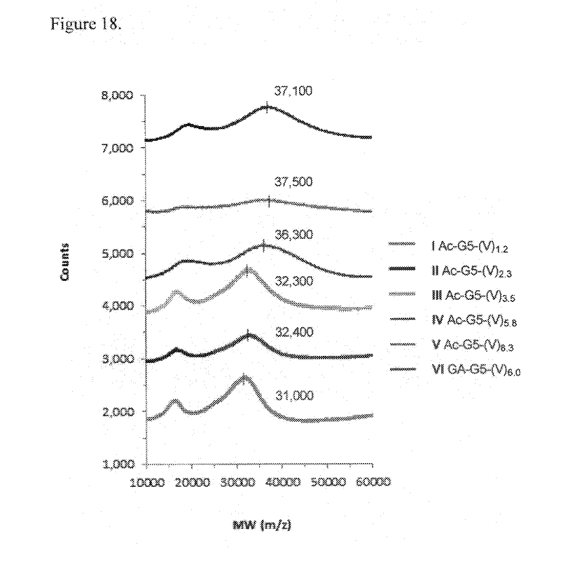

FIG. 18 shows MALDI TOF mass spectra of vancomycin (V)-conjugated G5 PAMAM dendrimers, I-V Ac-G5-(V)ₙ (n=1.2, 2.3, 3.5, 5.8, 8.3) and VI GA-G5-(V)_{6.0}.

### Example 13

Materials. Unless noted otherwise, all reagents and solvents were purchased from Sigma-Aldrich that include vancomycin hydrochloride, diethylenetriaminepentaacetic dianhydride (DTPA), and fluorescein 5(6)-isothiocyanate (FITC; purity ˜90%). A fifth generation (G5) poly(amidoamine) (PAMAM) dendrimer was purchased as a 17.5% (w/w) methanol solution (Dendritech, Inc.). It was purified prior to use by membrane dialysis (MWCO 10000) against deionized water (see, e.g., Choi, S. K.; et al., Macromolecules 2011, 44, 4026-29; herein incorporated by reference in its entirety)). Iron oxide (Fe₃O₄) nanoparticles (<50 nm) were purchased from Sigma-Aldrich, and used as received.

Analytical methods. Each dendrimer conjugate was fully characterized by a number of standard analytical methods including high performance liquid chromatography (HPLC), gel permeation chromatography (GPC), matrix assisted laser desorption ionization-time of flight (MALDI TOF) mass spectrometry, and ¹H NMR spectroscopy (see, e.g., Choi, S. K.; et al., Macromolecules 2011, 44, 4026-29; Witte, A. B.; et al., Biomacromolecules 2012, 13, 507-16; each herein incorporated by reference in its entirety)). ¹H NMR spectroscopy was performed with a Varian nuclear magnetic resonance spectrometer at 500 MHz under a standard observation condition. Molecular weights of PAMAM dendrimers conjugated with vancomycin were determined by MALDI TOF mass spectrometry with a Waters TOfsPec-2E spectrometer (see, e.g., Choi, S. K.; et al., Chem. Commun (Cambridge, U. K.) 2010, 46, 2632-34; herein incorporated by reference in its entirety)). Bovine serum albumin mixed in sinapinic acid was used to calibrate the mass of the MALDI spectrometer, and data was processed using Mass Lynx 3.5 software. UV-vis absorption spectra were recorded on a Perkin Elmer Lambda 20 spectrophotometer. The purity of each dendrimer conjugate was assessed by HPLC on a Waters Acquity Peptide Mapping System equipped with a Waters photodiode array detector (see, e.g., Choi, S. K.; et al., Chem. Commun. (Cambridge, U. K.) 2010, 46, 2632-34; herein incorporated by reference in its entirety)). Each sample solution was run on a C4 BEH column (150×2.1 mm, 300 Å) connected to Waters Vanguard column, and the elution method was based on a linear gradient beginning with 98:2 (v/v) water/acetonitrile (with trifluoroacetic acid at 0.14 wt % in each eluent) at a flow rate of 1 mL/min. Gel permeation chromatography (GPC) experiments were used to measure polydispersity indices (PDI) of dendrimers (see, e.g., Thomas, T. P.; et al., Bioorg. Med. Chem. Lett. 2010, 20, 5191-94; herein incorporated by reference in its entirety)). Gel permeation chromatography (GPC) experiments were performed on an Alliance Waters 2695 separation module equipped with a 2487 dual wavelength UV absorbance detector (Waters Corporation), a Wyatt HELEOS Multi Angle Laser Light Scattering (MALLS) detector, and an Optilab rEX differential refractometer (Wyatt Technology Corporation). Details for the experimental method are already described elsewhere (see, e.g., Choi, S. K.; et al., Macromolecules 2011, 44, 4026-29; herein incorporated by reference in its entirety)) that include column types, a temperature control (25±0.1° C.), the mobile phase (0.1 M citric acid, 0.025% (w/w) sodium azide, pH 2.74), the flow rate (1 mL/min), and the sample concentration (10 mg/5 mL). The GPC analysis was carried out to determine weight-average molecular weight (M_{w}), and the number average molecular weight was calculated with Astra 5.3.14 software (Wyatt Technology Corporation) based on the molecular weight distribution. A value of polydispersity index (PDI=M_{w}/Mₙ) determined for the purified G5 PAMAM dendrimer (G5-NH₂) was 1.010.

Representative synthetic methods for G5-(Vancomycin)ₙ (Scheme 1). Preactivation of vancomycin: To a solution of vancomycin hydrochloride hydrate (64 mg, 43 μmol) dissolved in a mixture of anhydrous DMSO (2 mL) and DMF (1 mL) was added N,N-diisopropylethylamine (15 μL, 86 μmol), 1-hydroxybenzotriazole hydrate (HOBt, 6.6 mg, 49 μmol), and then benzotriazol-1-yloxytripyrrolidinophosphonium hexafluorophosphate (PyBOP; 23 mg, 44 μmol) in a sequence. After stirring for 40 min at room temperature, this solution was divided into three aliquots: 0.66 mL (aliquot A), 1.05 mL (aliquot B), 1.29 mL (aliquot C). Each of the three aliquots (containing the activated ester of vancomycin) was used immediately for conjugation to G5 PAMAM dendrimer (G5-NH₂) as follows.

Conjugates III-V Ac-G5-(V)ₙ (n=3.5, 5.8, 8.3): Typically, each aliquot was added to a rapidly stirred solution of G5-NH₂ (MW=26700 gmol⁻¹; 50 mg, 1.87 μmol) dissolved in methanol (12 mL) at the specific molar ratio of vancomycin to the dendrimer ([Vancomycin]/[G5-NH₂]=5, 8, 10). Then each mixture was stirred for 5 h at room temperature prior to the addition of N,N-diisopropylethylamine (59 μL, 339 μmol) followed by the addition of acetic anhydride (16 μL, 170 μmol). The final reaction mixture was stirred for an additional 1 h at room temperature, and concentrated in vacuo. The residue was diluted with 20 mL of phosphate-buffered saline (PBS, pH 7.4), loaded into a cellulose membrane dialysis tubing (MWCO 10 kDa), and dialyzed against PBS (2 L×2), and deionized water (2 L×2) over 2 days. The purified solution in each of the dialysis bags was collected and lyophilized to afford Ac-G5-(V)ₙ as white fluffy solid: 65 mg (from aliquot A), 75 mg (aliquot B), 87 mg (aliquot C). Purity of each conjugate Ac-G5-(V)ₙ was assessed by an analytical HPLC method: tᵣ=7.75-7.76 min (n=3.5, 5.8, 8.3), purity ≧99%. The number (n) of vancomycin molecules linked to each dendrimer conjugate Ac-G5-(V)ₙ was determined on a mean basis by the analysis of UV-vis and MALDI mass spectral data: n=3.5 (aliquot A condition); n=5.8 (aliquot B condition); n=8.3 (aliquot C condition). MALDI TOF mass spectrometry (m/z, gmol⁻¹): 32300 (III); 36300 (IV); 37500 (V). UV-vis for Ac-G5-(V)ₙ (PBS, pH 7.4): 282 nm (λₘₐₓ). Each conjugate was also characterized by the GPC method to determine its molecular weights (weight-averaged M_{w} and number-averaged Mₙ) and a polydispersity index value (PDI=M_{w}/Mₙ). III Ac-G5-(V)_{3.5}; M_{w}=30700 gmol⁻¹; PDI=1.043; IV Ac-G5-(V)_{5.8}: M_{w}=37800 gmol⁻¹, PDI=1.027; V Ac-G5-(V)_{8.3}; M_{w}=33200 gmol⁻¹, PDI=1.032. Representative ¹H NMR data (500 MHz, D₂O; Figure S4) for V Ac-G5-(V)_{8.3}: δ 7.8-7.6 (br s),* 7.4 (br s), 7.2-6.9 (br peak), 6.6-6.5 (br s), 6.3 (br s), 5.7 (br s), 5.5 (br s), 5.3 (br s), 5.2 (br s), 4.6 (br peak), 4.3-4.1 (br peak), 3.8-3.7 (br peak), 3.3-3.1 (br s; dendrimer), 3.0-2.2 (multiple peaks; dendrimer), 1.9 (s; N-Ac, dendrimer), 1.7 (br s), 1.6-1.5 (br peak), 1.4-1.2 (br peak), 0.9 (br s) ppm. *Acronyms: br (broad), s (singlet).

Conjugate VI GA-G5-(V)_{6.0} (Scheme 1). To a solution of G5-NH₂ (127 mg, 4.76 μmol) dissolved in methanol (25 mL) was added a solution of the PyBOP-treated vancomycin (8 mol equiv to the dendrimer; 2.67 mL), the activated ester form prepared as described in the Experimental Section. The reaction mixture was stirred for 5 h at room temperature prior to the addition of N,N-diisopropylethylamine (150 μL, 861 μmol), and glutaric anhydride (48 mg, 421 μmol). After stirring for 1 h at room temperature, the mixture was concentrated in vacuo, and the residue was diluted with 20 mL of phosphate-buffered saline (PBS, pH 7.4). This solutions was loaded into a cellulose membrane dialysis tubing (MWCO 10 kDa), and dialyzed against PBS (2 L×2), and deionized water (2 L×2) over 2 days. The purified solution in the dialysis bag was collected and lyophilized to afford VI GA-G5-(V)₆ as white fluffy solid (103 mg). The purity of VI was determined by an analytical HPLC method, and it was greater than 99%: tᵣ=7.84 min. The number (n=6) of vancomycin molecules attached to the dendrimer conjugate GA-G5-(V)ₙ was determined on a mean basis by the analysis of UV-vis and MALDI mass spectral data. MALDI TOF mass spectrometry (m/z; gmol⁻¹): 37100. UV-vis (PBS, pH 7.4): λₘₐₓ=282 nm (ε=6716 M⁻¹cm⁻¹ calculated on the basis of vancomycin). ¹H NMR (500 MHz, D₂O; Figure S4): weak signals: δ 7.6 (br s),* 7.4-7.2 (m), 7.0 (m), 6.4, 6.1, 5.6 (br s), 5.3, 5.2 (m); strong signals: 3.48 (s), 3.4 (m), 3.2 (br s), 3.0 (s), 2.7 (m), 2.5 (s), 2.4-2.3 (m); weak signals: 1.5 (br), 1.3-1.1 (br m), 0.76 (br s) ppm. *Acronyms: br (broad), s (singlet), m (multiplet).

Conjugate VII DTPA-G5-(V)_{6.1} (Scheme 1). This conjugate was prepared from G5-NH₂ (127 mg, 4.76 μmol) in a similar manner to 6 with a minor modification where diethylenetriaminepentaacetic (DTPA) dianhydride (151 mg, 423 μmol) replaced the glutaric anhydride as the capping reagent for a full surface modification after the vancomycin conjugation. Conjugate VII DTPA-G5-(V)_{6.1} was obtained as white fluffy solid (192 mg). HPLC analysis: tᵣ=7.71 min; purity ≧99%. GPC: M_{w}=62500 gmol⁻¹, PDI=1.088. UV-vis (PBS, pH 7.4): λₘₐₓ=282 nm. ¹H NMR (500 MHz, D₂O): weak signals: δ 7.6 (br s), 7.4-7.2 (m), 7.0 (br m), 6.4, 6.1, 5.6 (br s), 5.3-5.2 (m); strong signals: 3.48 (s), 3.4 (m), 3.2 (m), 3.0 (s), 2.7 (m), 2.6 (s), 2.4-2.3 (m); weak signals: 1.7, 1.5 (br), 1.3-1.1 (m), 0.76 (br s) ppm.

Conjugate VIII DTPA-G5-(V)_{6.1}-(FL)_{3.9} (Scheme 1). To a mixture of conjugate VII DTPA-G5-(V)_{6.1}(20 mg, 0.32 μmol) and 4′,5′-fluoresceindiamine (1.3 mg, 2.4 μmol; prepared as described elsewhere⁵), both dissolved in DMF (3 mL), was added DIPEA (37 μL, 0.21 mmol), HOBt (1.8 mg, 12 μmol), and PyBOP (2.1 mg, 6.0 μmol). After stirring the reaction mixture for 24 h at room temperature, the mixture was concentrated in vacuo, and the residue was diluted with 10 mL of phosphate-buffered saline (PBS, pH 7.4). This solution was dialyzed using a membrane dialysis tubing (MWCO 10 kDa) against PBS (2 L×2), and deionized water (2 L×2) over 2 days in the dark. Freeze-drying of the dialyzed solution afforded conjugate VIII DTPA-G5-(V)_{6.1}-(FL)_{3.9} as orange fluffy solid. HPLC analysis: tᵣ=7.71 min; purity ≧99%. UV-vis (PBS, pH 7.4): λₘₐₓ=509 (FI), 281 nm.

Conjugate IX Ac-G5-(V)_{6.3}-(FITC)_{1.8} (Scheme 1). To a solution of G5-NH₂ (32 mg, 1.20 μmol) dissolved in methanol (7 mL) was added a solution of the PyBOP-treated vancomycin (8 mol equiv to the dendrimer; 1.34 mL) prepared by the preactivation method described earlier. After stirring the reaction mixture for 20 h at room temperature, fluorescein isothiocyanate (FITC; 1.8 mg, 4.6 μmol) was added as solid. The mixture was stirred for 16 h at room temperature prior to the addition of N,N-diisopropylethylamine (44 μL, 253 μmol), and acetic anhydride (12 μL, 127 μmol). After stirring for 1 h at room temperature, the mixture was concentrated in vacuo, and the residue was diluted with 20 mL of phosphate-buffered saline (PBS, pH 7.4). This solution was dialyzed using a membrane dialysis tubing (MWCO 10 kDa) against PBS (2 L×2), and deionized water (2 L×2) over 3 days in the dark. Freeze-drying of the dialyzed solution afforded IX Ac-G5-(V)_{6.3}-(FITC)_{1.8} as orange fluffy solid. HPLC analysis: tᵣ=8.35 min; purity ≧99%. MALDI TOF mass spectrometry (m/z; gmol⁻¹): 36300. UV-vis (PBS, pH 7.4): λₘₐₓ=501 (FITC; ε=7,6630 M⁻¹cm⁻¹), 281 nm.

Synthesis of dendrimer conjugated with both Vancomycin and Polymyxin B. This dual-targeting conjugate was synthesized by the Mannich conjugation method as reported elsewhere (Long, D.; et al., J. Antibiot. 2008, 61, 603-614). To a solution of Ac-G5-(V)_{5.8} conjugate IV (50 mg) dissolved in water (5 mL) was added 0.01 M NaOH (0.086 mL) and an aqueous formaldehyde solution (3.7%; 0.08 mL). After stirring for 45 min at room temperature, polymyxin B (56 mg) dissolved in water (1 mL) was added to the treated dendrimer. The mixture was stirred for 1 day prior to purification using a membrane dialysis method (MWCO 10 kDa) against a phosphate-buffered saline solution and water. The purified solution was dried by lyophilization, affording white solid (47 mg). HPLC: retention time (tᵣ)=8.5 min (broad peak) vs. tᵣ=7.9 min for conjugate IV (relatively sharp peak).

Synthesis of dendrimer conjugated with polymyxin B GA-G5-(Polymyxin) by amide method. This Gram negative cell-targeting dendrimer conjugate was synthesized by the EDC-based amide conjugation method as follows. To a solution of G5 PAMAM dendrimer derivatized with glutaric acid G5-GA (see, e.g., Choi, S. K.; et al., Chem. Commun. (Cambridge, U.K.) 2010, 46, 2632-2634) (MW=40200 g/mol; 200 mg) suspended in anhydrous DMF (15 mL) was added N-hydroxysuccinimide (NHS; 124 mg), 4-(dimethylamino)pyridine (DMAP; 164 mg), and N-(3-dimethylaminopropyl)-N′-ethylcarbodiimide hydrochloride (EDC; 257 mg). This reaction mixture was stirred at room temperature until it became fully solubilized (12 hr). After stirring, polymyxin B sulfate (69 mg) suspended in DMF (1 mL) containing N,N-diisopropylethylamine (DIPEA; 0.032 mL) was added to the EDC/NHS-treated dendrimer (molar ratio of polymyxin B added to G5-GA =15). The reaction mixture was stirred for 12 hr and the dendrimer product GA-G5-(Polymyxin) was purified by starting with concentration in vacuo. The resulting residue was dissolved in 10 mL of phosphate buffered saline (PBS, pH 7.4) and loaded into a membrane dialysis tubing (MWCO 10 kDa). The dendrimer solution in the tubing was dialyzed against PBS solution (1×2 L) and deionized water (2×2 L) over 2 days. After the dialysis, the dendrimer solution was dried by lyophilization, affording GA-G5-(Polymyxin) as white solid (213 mg). Purity of the dendrimer conjugate was assessed by analytical HPLC: retention time (tᵣ)=9.4 min. The number (n) of polymyxin B molecules linked to each dendrimer conjugate was determined on a mean basis by analysis of MALDI mass spectral data: n=5. MALDI TOF mass spectrometry (m/z, gmol⁻¹): 46400. UV-Vis spectrometry (PBS, pH 7.4): 291 nm (λₘₐₓ). ¹H NMR data (500 MHz, DMSO-d₆): δ 8.4-7.8 (multiple peaks), 7.2 (broad singlet), 3.6-2.8 (broad peaks), 2.7-1.8 (multiple broad peaks), 1.8-1.6 (broad peaks), 1.5-0.6 (multiple broad peaks) ppm.

Synthesis of dendrimer conjugated with polymyxin B G5-(Polymyxin) by N-alkylation method. Another type of Gram negative cell-targeting dendrimer conjugate was synthesized by the N-alkylation method as follows. To a solution of G5 PAMAM dendrimer (MW=27600 g/mol; 100 mg) dissolved in methanol (12 mL) was added epibromohydrin (0.077 mL). This reaction mixture was stirred at room temperature for 24 hr prior to the addition of polymyxin B sulfate (75 mg) dissolved in 1 M NaOH solution (0.65 mL) (molar ratio of polymyxin B added to G5-NH₂=15). After stirring the reaction mixture for 24 hr at room temperature, ethanolamine (0.088 mL) was added to the mixture to quench the reaction by reacting with excess epoxides. The resulting mixture was stirred at the same temperature for 12 hr, and concentrated in vacuo. The residue was dissolved in water (˜20 mL), loaded into a membrane dialysis tubing (MWCO 10 kDa) and dialysed against deionized water (3×2 L) over 2 days. The dialyzed solution was dried by lyophilization, affording the dendrimer conjugate as white solid. This dendrimer polymyxin conjugate G5-(Polymyxin) was characterized by standard analytical methods as described above. HPLC analysis: retention time (tᵣ)=6.2 min.

Synthesis of dendrimer-coated IONP-VI and IONP-VII (Scheme 2). Amine-terminated IONP: Iron oxide nanoparticles (Fe₃O₄, <50 nm, 3 g) were placed in a glass flask containing toluene (100 mL). To this suspension was added a catalytic amount of acetic acid (0.1 mL), and then followed by the addition of (3-aminopropyl)trimethoxysilane (APMS) while shaking the mixture mechanically. After shaking the mixture overnight at room temperature, the iron oxide particles were collected by centrifugation (4500 rpm). It was rinsed four times with toluene, each time according to a rinsing protocol that comprises of: i) suspending in toluene (50 mL), ii) sonication (30 s), iii) spinning (4500 rpm). Subsequently, drying under a nitrogen flow afforded APMS-coated iron oxide particles (3.25 g).

IONP-VI and IONP-VII: As a first step in coupling of vancomycin-conjugated dendrimer nanoparticles to the amine-terminated IONPs prepared above, each dendrimer conjugate was prepactivated by an EDC method as follows. To a solution of VI GA-G5-(V)₆ (MW=37100 gmol⁻¹) or VII DTPA-G5-(V)_{6.1} (MW=62500 gmol⁻¹), each (10 mg) dissolved in a mixture of DMSO (2 mL) and DMF (3 mL), was added 4-dimethylaminopyridine (DMAP; 50 mol equiv to dendrimer), N-hydroxysuccinimide (NHS; 100 equiv), and finally N-(3 -dimethylaminopropyl)-N′-ethylcarbodiimide hydrochloride (EDC; 100 equiv). After stirring each reaction mixture at room temperature for 12 h, the activated dendrimer conjugate reacted with APMS-coated IONP (300 mg) by adding the IONP to the conjugate solution. The mixture was mechanically shaken at room temperature for 6 h, and then diluted with water (1 mL) prior to the addition of a second portion of EDC (100 mg each). The final mixture was shaken at room temperature for an additional 12 h. Isolation of IONP-VI or IONP-VII started with dilution of each reaction mixture with 14 mL of water and followed by centrifugation at 4500 rpm. A dark brown pellet was collected by carefully decanting the supernatant, and it was resuspended in water (14 mL). After a short period of sonication (30 s), the mixture was centrifuged and the pellet was collected. This rinsing process continued but by using 70% aq. EtOH (14 mL). Each pellet (IONP-VI or IONP-VII) was collected and stored as a suspension in 70% aq. EtOH (˜25 mg/mL): UV-vis (70% aq. EtOH): λₘₐₓ=282 nm. The fraction of dendrimer conjugates VI (or VII) attached to IONP was estimated by a colorimetric analysis of unreacted dendrimer conjugates left in the supernatant using UV-vis spectrophotometry: Fr_{free}=[conjugate]ₛᵤₚₑᵣₙₐₜₐₙₜ÷[conjugate]_{added}; Fr_{attached}=(1−Fr_{free})≈0.75. This fractional analysis was used to calculate the ratio of the conjugate attached to IONP on a weight basis: (wt_{conjugate}÷wt_{IONP})×100(%)≈2.5%.

In addition, this method was applied for covalent attachment of dendrimer conjugated with other bacteria targeting agents (e.g., Polymyxin B alone, or a combination of Vancomycin and Polymyxin B) to the IONP-NH₂, yielding an IONP coated with this dual-targeting dendrimer.

Synthesis of IONP conjugated with G5 dendrimer-polymyxin. This polymyxin B-attached dendrimer conjugate was prepactivated by an EDC method developed for the coupling of vancomycin-conjugated dendrimer nanoparticles to the amine-terminated IONPs prepared above. To a solution of GA-G5-(Polymyxin)₅ (MW=46400 gmol⁻¹; 50 mg) dissolved in DMF (15 mL) was added 4-(dimethylamino)pyridine (DMAP; 36 mg), N-hydroxysuccinimide (NHS; 27 mg), and finally N-(3 -dimethylaminopropyl)-N′-ethylcarbodiimide hydrochloride (EDC; 56 mg). After stirring the reaction mixture at room temperature for 12 hr, the activated dendrimer conjugate was treated with APMS-coated IONP (500 mg) by adding this IONP to the EDC/NHS-activated solution. The mixture was mechanically shaken at room temperature for 24 hr, and then divided equally into two lots (each ˜7.5 mL). One lot was stirred without any further treatment for an additional 24 hr period. To the other lot was added fluorescein isothiocyanate (FITC; 8 mg) to prepare fluorescent magnetic nanoparticles, and the mixture was shaken at room temperature for 24 hr. Isolation and purification of each IONP conjugated with GA-G5-(Polymyxin) started with dilution of the reaction mixture with 8 mL of water and followed by centrifugation at 4500 rpm for 10 min. A black brown pellet was collected separately from each lot by careful decanting of the supernatant, and was resuspended in water (10 mL). After a short period of sonication (30 s), the suspension was centrifuged again and the pellet was recollected. This rinsing process was repeated by using 70% aq. EtOH twice (10 mL×2). Each pellet was collected and stored as a suspension in 70% aq. EtOH (˜25 mg/mL): amount isolated=274 mg of IONP-G5(Polymyxin); 250 mg of FITC-IONP-G5(Polymyxin). UV-Vis (70% aq. EtOH): λₘₐₓ=285 nm for IONP-G5(Polymyxin); λₘₐₓ=265, 500 nm for FITC-IONP-G5 (Polymyxin). The fraction of dendrimer polymyxin conjugate particles attached to IONP was estimated by UV-vis analysis of unreacted dendrimer conjugate particles left in the supernatant: Fr_{free}=[conjugate]ₛᵤₚₑᵣₙₐₜₐₙₜ÷[conjugate]added; Fr_{attached}=(1−Fr_{free})≈0.72 for IONP-G5(Polymyxin); 0.56 for FITC-IONP-G5(Polymyxin). This fractional analysis was used to calculate the ratio (wt/wt) of the conjugate attached to IONP on a mean basis: (wt_{conjugate}÷wt_{IONP})×100(%)≈6.8% for IONP-G5(Polymyxin); 5.3% for FITC-IONP-G5(Polymyxin).

Dendrimer-drug complexation. This example describes complexation of vancomycin-conjugated dendrimer VI with additional antibacterial agents. To a solution of GA-G5-V_{6.0} conjugate VI (10 mg) dissolved in 2 mL of water was added polymyxin B sulfate (37 mg; [Drug]/[Dendrimer]=100) dissolved in 1.5 mL of water. After incubation at room temperature for 30 min, the complex solution was transferred to a centrifugal dialysis tube (Amicon, MWCO10 kDa), and spinned down until it was concentrated to 350 microL. Water was added to the concentrate to make a final volume of 3.5 mL, and spinned until it was 350 microL. This spinning procedure was repeated one more time, and after this round, the concentration of the free (uncomplexed) drug was 1000-fold diluted. The complex solution in the upper tube was collected and lyophilized, yielding GA-G5-V_{6.0}/polymyxin B complex as white solid. This method was also applied for complexation of GA-G5-V_{6.0} with gentamicin or silver agent (using AgNO₃).

SPR spectroscopy. SPR experiments were carried out on a Biacore® X instrument (Pharmacia Biosensor AB, Uppsala, Sweden) following the protocol, as reported elsewhere.^{31, 57-58} Cell wall models for Gram-positive bacteria were generated by immobilizing cell wall precursor peptides, either N^{α}-Ac-Lys-(D)-Ala-(D)-Ala (Sigma-Aldrich), or N^{α}-Lys-(D)-Ala-(D)-Lac (Bachem), to a CMS sensor chip (Biacore). Thus each chip surface represents a vancomycin-susceptible or vancomycin-resistant cell wall model, respectively. As an illustration, the carboxymethylated dextran-coated layer of the chip was preactivated by injection (flow rate=10 μL/min; volume=50 μL) of a 1:1 mixture of EDC (0.4 M) and NHS (0.1 M), each dissolved in H₂O. Immediately after this preactivation step, each peptide solution prepared in H₂O (70 μL, 20 mg/mL, pH=9-10) was injected for covalent attachment of the peptide to the dextran surface, and followed by injection of ethanolamine (50 μL, 1 M, pH 8.0) to convert unreacted activated esters to neutral amides on the surface. The immobilization process resulted in a net increase in response unit (RU) of 120 (0.12 ng/mm² equivalent to ˜2.2×10¹¹ molecules/mm²) for each peptide. A reference flow cell in each chip was then prepared in a similar way but without injecting the peptide. SPR studies were carried out by injecting an analyte solution, each prepared in HBS-EP buffer, at a flow rate of 20 μL/min for free vancomycin molecule as a positive control, or 10 μL/min for dendrimer conjugates G5-(V)ₙ. After each run, the surface of the chip was regenerated by repeated injections of an N^{α}-Ac-Lys-(D)-Ala-(D)-Ala solution (10 mg/mL) until the baseline of the sensorgram reaches an initial level.

For kinetic analysis, each SPR sensorgram acquired from flow cell 1 (RU₁; peptide immobilized) was corrected for non-specific binding by subtraction of the reference sensorgram from flow cell 2 (RU₂), as illustrated in FIG. 6 (ΔRu=R₁−RU₂). Kinetic binding parameters, the on-rate (kₒₙ), and the off-rate (k_{off}), were extracted by fitting each sensrogram separately using the Langmuir kinetic model (see, e.g., Hong, S.; et al., Chem. Biol. (Cambridge, Mass., U.S.) 2007, 14, 107-115; Li, M.-H.; et al., et al., Eur. J. Med. Chem. 2012, 47, 560-572; Ober, R. J.; et al., Anal. Biochem. 2003, 312, 57-65; MacKenzie, C. R.; et al., J. Biol. Chem. 1996, 271, 1527-1533; each herein incorporated by reference in its entirety)). Given the nature of dendrimer distribution associated with each conjugate, the fitting of each dissociation curve by employing a single exponential decay function was problematic. It was assumed that the decay curve represents a summation of two or more independent dissociation kinetics, each attributable to a group of dendrimer populations bound with a specific functional valency. After thorough kinetic analysis, it was possible to distinguish two dissociation populations (faster, and slower) by the curve fitting based on a linear combination of dual exponential decay functions (Eq 1). Out of two off-rate constants (k_{off,1}, k_{off,2}) obtained, the slower rate constant accounted for the predominantly larger fraction (80-90%) of the decay curve and it was used to determine the on-rate constant (kₒₙ) in the association phase (Eq 2). These two rate constants (k_{off}, kₒₙ) are all reported in the Table 2 along with their equilibrium dissociation constants (K_{D}).

Dissociation phase: RU(t)=C₁RUₜ₌₀×exp^{-(k}^{_{off,1}}^{)t}+C₂RUₜ₌₀×exp^{-(k}^{_{off,2}}^{)t} (Eq. 1)

Association phase: RU(t)=C₃(1−exp^{-(k}^{ₛ}^{)t})+RUₜ₌₀ (Eq. 2)

Fitting parameters noted in each equation are defined as follows: RU(t)=a response unit (ΔRU) at a specific time (t); RUₜ₌₀=RU at t=0 (in either a dissociation or association phase); C₁, and C₂=a coefficient that determines the weight of each dissociation component; C₃=kₒₙ[conjugate]RU_{A}/(kₒₙ[conjugate]+k_{off}); kₛ=(kₒₙ[conjugate]+k_{off}). An equilibrium dissociation constant K_{D}(=k_{off}/kₒₙ) reported for each G5-(V)ₙ refers to a mean value obtained from multiple independent measurements (n≧5) per conjugate.

Confocal microscopy. Staphylococcus aureus bacteria (ATCC 4012) were purchased from ATCC. The cells were stored in a frozen (−80° C.) suspension of 10% glycerol in nutrient broth with fetal bovine serum (FBS) until it was used. Prior to the study, a small amount of partially thawed bacterial suspension was spread on a nutrient agar plate (Thermo Fisher Scientific IP-265) and incubated at 37° C. The plated bacteria were harvested from the solid media after 48 h incubation and washed in sterile PBS (centrifugation at 9000 rpm for 10 min) For confocal microscopy, the bacterial cells were suspended in PBS (10⁶ CFU/ml) and treated with 86 μM of either the conjugate VIII DTPA-G5-(V)_{6.1}-(Fl)_{3.9} or IX Ac-G5-(V)_{6.3}-(FITC)_{1.8}, or the non-targeted control dendrimer, GA-G5-(FITC) for 30 min at room temperature. The treated cells were then washed with PBS and fixed in 4% paraformaldehyde in PBS for 10 min at room temperature. Cells were rinsed multiple times with PBS and then stained with Syto® 59, a cell-permeable fluorescent DNA binding molecule (λₑₘ=645 nm; red). Cells were washed with PBS and resuspended in PBS. Each bacterial cell suspension was applied onto a chambered cover glass, and allowed for air dry before mounting the slide in Prolong Gold Antifade (Life Technologies, Carlsbad, Calif.). Images were acquired using a Zeiss LSM 510-META laser scanning confocal microscope (Carl Zeiss Microscopy, LLC, Thornwood, N.Y.) equipped with argon and helium-neon lasers. Fluorescence was measured using 488 nm (green) and 543 (red) excitation lines, and imaged at a 63-fold magnification.

Turbidity assay. The assay was performed as previously described with minor modifications (see, e.g., Myc, A.; Horn, R.; Hamouda, T.; Baker, J. R., Fungicidal Effect of a “Hybrid” Surfactant Lipid Preparation (SLP) on Candida Ssp. In 99th General Meeting of American Society for Microbiology, Chicago, Ill., 1999; herein incorporated by reference in its entirety)). A stock solution of vancomycin or an equimolar concentration of a vancomycin conjugate was prepared in a brain-heart infusion (BHI) medium and 2-fold serial dilutions were made for each test compound on a 96-well flat bottom plate (100 μL per well). One hundred microliter of an inoculum of staphylococcus aureus bacteria was added to each well at the concentration of 5×10⁶ CFU/mL. After incubation of the plates at 37° C. for 24 h, bacterial growth was examined by optical microscopy and reading an optical density (O. D.) value at 650 nm by an ELISA plate reader.

Bacterial isolation by IONPs. On the day of the experiment for bacterial isolation, each IONP suspension was agitated and an aliquot needed for the experiment was taken out. It was centrifuged at 9000 rpm for 10 min and washed with sterile PBS. The IONPs were resuspended in the PBS solution to a final concentration (2.5 mg/mL). A suspension of staphylococcus aureus (ATCC 4012) bacteria in PBS (˜1×10⁸ CFU/mL) was split into several 1 mL aliquots in culture test tubes. Each test IONP (100 uL), prepared at the concentration of 2.5 mg/ml, was added to an aliquot of bacteria and the culture tube was incubated at room temperature for 15 min with occasional agitations. Subsequently, each tube was placed close to a magnet and left for 30 s. Then the supernatant was gently removed from each culture tube and saved. One mL of the sterile PBS solution was replenished to the tube and IONPs were gently washed. After washing twice, the test tube was removed from the magnet and IONPs were resuspended in PBS. A series of ten-fold dilutions were made for each supernatant and separately for each IONP isolated. The diluted sample (50 μL each) was placed on the agar plate, and the plate was incubated for 48 h at 37° C. Distinguishable colonies were counted from each plate, and the mean number of bacterial colonies was estimated from the plot against dilution factors. Efficiency for isolating bacterial cells by each IONP was compared to the control level obtained by the same experiment performed with bacteria untreated.

In vitro cytotoxicity assay. Cytotoxicity of select vancomycin-conjugated PAMAM dendrimers to mammalian cells was evaluated in two cell lines including KB cells, a sub-line of the cervical carcinoma HeLa cells, and mouse melanoma B16-F10 cells (see, e.g., Thomas, T. P.; et al., Biomacromolecules 2009, 10, 3207-3214; herein incorporated by reference in its entirety)). After incubation of the cells for 3 days in the microtiter plate, each conjugate was added in a concentration-dependent manner. The number of cells grown was quantified by a colorimetric XTT assay (sodium 3-[1-(phenylaminocarbonyl)-3,4-tetrazolium]-bis(4-methoxy-6-nitro)benzene sulfonic acid hydrate; Roche Mol. Biochem.) (see, e.g., Roehm, N. W.; et al., J. Immunol. Methods 1991, 142, 257-265; herein incorporated by reference in its entirety)) by reading absorbance at 492 nm relative to the reference value at 690 nm using on an ELISA reader (Synergy HT, BioTek).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in dendrimer synthesis, drug delivery, or related fields are intended to be within the scope of the following claims.

1-45. (canceled)

46. A method for treating a Gram-positive and/or Gram-negative bacterial disorder, comprising administering to a subject suspected of having a Gram-positive and/or Gram-negative bacterial disorder a composition comprising dendrimer nanoparticles conjugated with Vancomyocin and/or Polymyxin B or E molecules, wherein said dendrimer nanoparticles bind with Gram-positive and/or Gram-negative bacteria, wherein said binding results in the amelioration of symptoms related to the Gram-positive and/or Gram-negative bacterial disorder, wherein said subject is a mammal.

47. The method of claim 46,
wherein said dendrimer nanoparticles are conjugated with two or more Vancomycin molecules and/or two or more Polymyxin B or E molecules,
wherein said Gram-positive or Gram-negative bacteria comprise vancomycin-resistant Gram-positive bacteria or polymyxin B or E-resistant Gram-negative bacteria, and
wherein said Gram-positive or Gram-negative bacterial disorder is selected from the group consisting of pneumonia, endocarditis, bacteremia, sepsis and toxemia.

48. The method of claim 47, wherein said two or more is an average of 2.3, 3.5, or 5.8.

49. The method of claim 46, wherein said composition comprises iron oxide nanoparticles, wherein said iron oxide particles are coated with said dendrimer nanoparticles.

50. The method of claim 46, wherein said binding comprises a binding of said Vancomyocin and/or Polymyxin B or E molecules with said Gram-positive and/or Gram-negative bacteria, respectively.

51. The method of claim 46, wherein said compositions are co-administered with additional agents configured for treating Gram-positive and/or Gram-negative bacterial disorders.

52. The method of claim 46, wherein said dendrimer is a PAMAM dendrimer.

53. A method for detecting the presence of Gram-positive and/or Gram-negative bacteria in a sample, comprising administering to said sample a composition comprising dendrimer nanoparticles conjugated with Vancomyocin and/or Polymyxin B or E molecules molecules and imaging agents, wherein upon said administering said dendrimer nanoparticles bind with said Gram-positive and/or Gram-negative bacteria, and detecting the imaging agents within said sample bound with said Gram-positive and/or Gram-negative bacteria.

54. The method of claim 53,
wherein said dendrimer nanoparticles are conjugated with two or more Vancomycin molecules and/or two or more Polymyxin B or E molecules,
wherein said Gram-positive or Gram-negative bacteria comprise vancomycin-resistant Gram-positive bacteria or polymyxin-resistant B or E Gram-negative bacteria.

55. The method of claim 54, wherein said two or more is an average of 2.3, 3.5, or 5.8.

56. The method of claim 53, wherein said composition comprises iron oxide nanoparticles, wherein said nanoparticles are coated with said dendrimer nanoparticles.

57. The method of claim 53, wherein said imaging agent is selected from the group consisting of an MRI contrast agent, Gd-DTPA, and/or a molecular dye, fluorescein isothiocyanate (FITC), 6-TAMRA, acridine orange, and cis-parinaric acid.

58. The method of claim 53, wherein said sample is a liquid sample selected from the group consisting of a water sample, a food sample, a pharmaceutical sample, a blood sample, or a blood product sample.

59. The method of claim 58, wherein said method is used for one or more of the following:
to screen the food sample for Gram-positive and/or Gram-negative bacterial contamination,
to screen the blood sample for Gram-positive and/or Gram-negative bacterial contamination.
to screen the blood product sample for Gram-positive and/or Gram-negative bacterial contamination,
to screen the water sample for Gram-positive and/or Gram-negative bacterial contamination,
to screen the pharmaceutical sample for Gram-positive and/or Gram-negative bacterial contamination, and
to screen the food sample for Gram-positive and/or Gram-negative bacterial contamination.

60. The method of claim 53, wherein said dendrimer is a PAMAM dendrimer.

61. A method for sequestering Gram-positive and/or Gram-negative bacteria within a sample, comprising administering compositions comprising iron oxide nanoparticles coated with dendrimer nanoparticles conjugated with Vancomycin or Polymyxin B or E alone, or in combination to said sample such that said iron oxide nanoparticles bind with said Gram-positive and/or Gram-negative bacteria, and sequestering said iron oxide nanoparticles bound with said Gram-positive and/or Gram-negative bacteria through administration of a magnetic field and/or centrifugation to said sample.

62. The method of claim 61, wherein said dendrimer nanoparticles are conjugated with two or more Vancomycin molecules and/or two or more Polymyxin B or E molecules.

63. The method of claim 62, wherein said two or more is an average of 2.3, 3.5, or 5.8.

64. The method of claim 61, wherein said Gram-positive or Gram-negative bacteria comprise vancomycin-resistant Gram-positive bacteria or polymyxin B or E resistant Gram-negative bacteria.

65. The method of claim 61, wherein said sample is a liquid sample selected from the group consisting of a water sample, a food sample, a pharmaceutical sample, a blood sample, or a blood product sample.

66. The method of claim 65, wherein said sequestering alleviates one or more of the following:
a Gram-positive and/or Gram-negative bacterial contamination of the food sample,
a Gram-positive and/or Gram-negative bacterial contamination of the blood sample,
a Gram-positive and/or Gram-negative bacterial contamination of the blood product sample,
a Gram-positive and/or Gram-negative bacterial contamination of the water sample, and
a Gram-positive and/or Gram-negative bacterial contamination of the pharmaceutical sample.

67. The method of claim 61, wherein said dendrimer is a PAMAM dendrimer.
